(19) United States
(12) Patent Application Publication
(10) Pub. No.: US 2023/0180741 A1
(43) Pub. Date: Jun. 15, 2023

(54) DEHP-Free Blood Storage and Methods of Use Thereof

(71) Applicant: Hemanext Inc., Lexington, MA (US)

(72) Inventor: Samuel O. SOWEMIMO-COKER, Dix Hills, NY (US)

(73) Assignee: Hemanext Inc., Lexington, MA (US)

(21) Appl. No.: 17/924,348

(22) PCT Filed: May 12, 2021
(86) PCT No.: PCT/US2021/032091

§ 371 Date: Nov. 9, 2022

Related U.S. Application Data
(60) Provisional application No. 63/024,190, filed on May 13, 2020.

(51) Int. Cl. A01N 1/02; A61M 1/02

(52) U.S. Cl. CPC A01N 1/0226; A01N 1/0263; A61M 1/0272

(57) ABSTRACT

The present disclosure relates to carbon dioxide permeable containers for storing blood and methods for the improved preservation of whole blood and blood components. The improved devices and methods for the collection of blood and blood components provide for whole blood and blood components having reduced levels of carbon dioxide and the elimination of the plasticizer DEHP. The devices and methods provide for the preparation of carbon dioxide depleted blood and blood components for storage that improves the overall quality of the transfused blood and improves health outcomes inpatients and reduces risks associated with DEHP. The devices and methods also provide for maintenance of low oxygen content in blood and blood components during storage. Compositions comprising a blood product and an additive solution are also disclosed.

### CROSS REFERENCE TO RELATED APPLICATION

This application claims the benefit of U.S. Provisional Application No. 63/024,190, filed May 13, 2020, which is hereby incorporated by reference.

### FIELD OF THE INVENTION

The present disclosure relates to containers for the storage and carbon dioxide reduction of blood and blood products. The present disclosure also relates to methods of managing carbon dioxide during storage for the improved preservation of blood and blood components. The present disclosure further relates to methods and devices for the preparation of di-2-ethylhexyl phthalate (DEHP) free (DEHP-free) stored blood.

### BACKGROUND OF THE INVENTION

Supplies of blood and blood components are currently limited by available storage systems used in conventional practices of storing blood. Conventional storage practices include collection of blood into anticoagulant solutions, preparation of red blood cell concentrates through the removal of plasma, leukoreduction, and storage of red blood cell concentrates in an additive solution. Using the conventional storage systems, packed red blood cell preparations expire after a period of about 42 days of refrigerated storage at approximately 4° C.

Currently, red blood cells are the most widely transfused blood component worldwide. This makes development of storage procedures to increase the storage time of blood while minimizing storage-based lesions imperative.

During storage, the accumulation of biochemical and biophysical changes (collectively called storage lesions (“lesions”)) progressively reduce the quality of red blood cells (RBCs) during storage. See Yoshida T., et al. “Red blood cell storage lesion: causes and potential clinical consequences,” Blood Transfus. 27(17): 27-52 (2019); Zimring J C., “Established and theoretical factors to consider in assessing the red cell storage lesion,” Blood 125(4):2185-2189 (2015); Donadee C, et al., “Nitric oxide scavenging by red blood cell microparticles and cell-free hemoglobin as a mechanism for the red cell storage lesion,” Circulation. 124(4):465-476 (2011). Changes in such in vitro measured parameters as reduced metabolite levels (e.g., adenosine triphosphate (ATP) and 2,3 diphosphoglycerate (2,3-DPG)), increased levels of free hemoglobin, hemolysis, non-transferrin bound iron, microparticles, and phosphatidylserine exposure are among the biochemical storage lesions. Physiologically, red blood cells experience reduced deformability during storage.

Storage lesions are associated with reduced in vivo recovery and blood quality. Clinical studies suggest that storage-induced changes may adversely affect clinical outcomes in different patient populations when these cells are transfused. See Triulzi D J, et al. “Clinical studies of the effects of blood storage on patient outcomes.” Transfus Apher Sci. 43(1):95-106 (2010); Voorhuis F T, et al. “Storage time of red blood cell concentrates and adverse outcomes after cardiac surgery: a cohort study.” Ann Hematol. 92(12):1701-1706 (2013); and Spinella P C, et al. “Duration of red blood cell storage is associated with increased incidence of deep vein thrombosis and in hospital mortality in patients with traumatic injuries”. Crit Care. 13(5):R151 (2009).

Over the years, several strategies have been explored to reduce storage lesions during refrigeration of RBCs. See Lagerberg J W, et al. “Prevention of red cell storage lesion: a comparison of five different additive solutions.” Blood Transfus. 15(5): 456-462 (2017); D'Alessandro A, et al. “Metabolic effect of alkaline additives and guanosine/gluconate in storage solutions for red blood cells.” Transfusion. 58(8):1992-2002 (2018); and Stowell S R, et al., “Addition of ascorbic acid solution to stored murine red blood cells increases post-transfusion recovery and decreases microparticles and alloimmunization,” Transfusion. 53(10):2248-57 (2013).

One contributor to storage lesions in red blood cells for transfusion is oxidative damage to lipids and proteins by reactive oxygen species (ROS) including hydroxy, peroxyl and alkoxy radicals formed from oxygen present in the blood during storage. See Yoshida T, et al. “Extended storage of red blood cells under anaerobic conditions.” Vox Sang. 92:22-31 (2007); and Yoshida T, et al. “Anaerobic storage of red blood cells.” Blood Transfus. 8(4):220-36 (2010). Therefore, one approach that has been shown to improve the quality and in vivo recovery of stored RBCs is the removal of O₂ from the RBCs prior to storage and maintaining the anaerobic condition throughout the storage duration. Two forms of anaerobic storage have been evaluated for maintaining blood cell quality during storage. In one approach, the oxygen is reduced prior to the initiation of storage (e.g., deplete and store). See Yoshida et al. “Extended storage of red blood cells under anaerobic conditions.” Vox Sang. 92:22-31 (2007); and Yoshida et al., “Anaerobic storage of red blood cells,” Blood Transfus. 8(4):220-36 (2010); Yoshida et al., “The effects of additive solution pH and metabolic rejuvenation on anaerobic storage of red cells,” Transfusion. 48(10):2096-2105 (2008); Dumont et al., “Anaerobic storage of red blood cells in a novel additive solution improves in vivo recovery,” Transfusion. 49(3):458-464 (2009); D'Alessandro et al., “Hypoxic storage of red blood cells improves metabolism and post-transfusion recovery,” Transfusion. [published online ahead of print (February 2020)]; International Publication Nos. WO 2016/172645 to Yoshida, T., et al. and WO 2016/145210 to Wolf, M., et al. Storing blood under oxygen depleted conditions increases the levels of ATP and 2,3-DPG compared to conventionally stored blood at similar times and maintains hemolysis levels below 0.8% after 42 days. An alternate approach involving the storage of packed red blood under oxygen free conditions without pre-storage deoxygenation has been studied (e.g., store and deplete) See, Hogman et al., “Effects of oxygen on red cells during liquid storage at +4 degrees C.,” Vox Sang. 51(1):27-34 (1986). While store and deplete methods are more convenient, they have not been able to match the quality of deplete and store approaches until the present specification.

Further studies demonstrate that carbon dioxide levels directly contribute to enhanced 2,3-diphosphoglycerate acid (DPG) levels in RBCs when combined with deoxygenation in deplete and store methods. See International Publication No. WO 2012/027582 (“the '582 publication”). The '582 publication further shows that 2,3 DPG enhancement is independent of pH, the condition that was thought to be controlling. See Dumont et al., “CO2 dependent metabolic modulation in red blood cells stored under anaerobic condition,” Transfusion 56(2):392-403 (2016).

A variety of storage solutions have been developed to reduce the deleterious effects of storage lesions and improve RBC quality and clinical outcomes. Changes in storage solutions are known to increase the production of ATP, 2,3-DPG, and reduce hemolysis. Efforts to reduce oxidative damage to the RBCs include incorporation of antioxidants in storage formulations. See Högman et al., “Storage of red blood cells with improved maintenance of 2,3-bisphosphoglycerate,” Transfusion. 46(9):1543-52 (2006); Radwanski et al., “Red cell storage in E-Sol 5 and Adsol additive solutions: paired comparison using mixed and non-mixed study designs,” Vox Sang 106(4):322-329 (2014); Cancelas et al., “Additive solution-7 reduces the red blood cell cold storage lesion,” Transfusion 55(3):491-498 (2015); Lagerberg et al., “Prevention of red cell storage lesion: a comparison of five different additive solutions,” Blood Transfus. 15(5):456-462 (2017); and Pallotta et al, “Storing red blood cells with vitamin C and N-acetylcysteine prevents oxidative stress-related lesions: a metabolomics overview,” Blood Transfus. 12(3):376-387 (2014).

An unexpected benefit of the development of plastic storage containers, particularly PVC, is the protective effect of the plasticizer DEHP, used in most PVC based blood storage bags. See U.S. Pat. No. 4,386,069 issued to Estep. However, recently, concerns that DEHP may act as an endocrine disruptor has led to efforts by authorities to consider removing DEHP from blood bags. However, the removal of DEHP has proved problematic as the presence of DEHP masks or reduces lesions. See D'Alessandro, A., et al. “Rapid detection of DEHP in packed red blood cells stored under European and US standard conditions.” Blood Transfus. 14(2): 140-144 (2016). Removing plasticizers from the storage systems results in significant changes in the following red cell qualities: 1) increase in red blood cell hemolysis; 2) decrease in the shelf life of red blood cells in additive solutions to less than the current 42 days; 3) decrease in red cell in vivo recovery; 4) increases red cell osmotic fragility; 5) increase in microvesicle formation; 6) decrease red cell deformability; and 7) decrease red cell morphology scores. Therefore, replacement of DEHP in blood storage bags poses significant technical challenges because of each of these benefits of DEHP maintain the stability and quality of red blood cells during extended storage at refrigerated temperatures. The present disclosure overcomes all of the technical challenges and produces red blood cells with superior quality and red cell hemolysis that meet regulatory requirement of less than 0.8% at the end of storage.

The prevention and reduction of storage lesions remains a challenge. The growing interest in removing DEHP from the supply requires the development of new storage containers and methods that replace the benefits previously provided by DEHP. Further, development of additive solutions that perform well and safely when DEHP is removed and are adapted to the storage conditions are needed.

In light of current technology, there is a need to improve the quality of blood and blood components such as red blood cells that are to be stored and to extend the storage life of such blood and blood components in advance of transfusion to help minimize morbidity associated with transfusions. In order to conform with regulatory requirements and to ensure reliability, the preparation and processing of the red blood cells must be completed within a limited time period. Further, the process of preparing reduced carbon dioxide blood and blood components must not introduce lesions, including but not limited to, hemolysis of the blood. Finally, there is a need for methods and devices that are compatible with existing anticoagulant and additive solutions to yield improved quality blood and blood components.

To address such needs and others, the present disclosure includes and provides devices, compositions, and methodology for the preservation of blood and blood components in which the preparation of carbon dioxide, or carbon dioxide and oxygen reduced blood and blood components is initiated at the donor collection stage.

In the present specification, storage bags for blood comprising DEHP-free gas permeable biocompatible polymers are constructed and used in methods of storing blood products with reduced levels of CO₂ and O₂ compared to conventionally stored cells. The methods and containers of the present specification improve anaerobic storage approaches with initial depletion of oxygen below 20% prior to storage. The present specification shows for the first time that reducing CO₂ levels and preventing oxygenation during storage maintains RBC health superior to both conventional storage and as well as deplete and store anaerobic methods.

### SUMMARY OF THE INVENTION

The present disclosure provides for and includes a method for the storage of a blood product comprising: obtaining a blood product having a % SO₂ of greater than 30%; adding an additive solution to the blood product to prepare a storable blood product; and storing the storable blood product in a di-2-ethylhexyl phthalate free (DEHP-free) blood compatible (BC) carbon dioxide permeable bag comprising a gas permeability for carbon dioxide of at least 0.62 centimeters cubed per centimeters squared (cm³/cm²) at about 1 atm at 25° C.

The present disclosure provides for and includes a container for storing blood comprising a DEHP-free carbon dioxide permeable and oxygen impermeable material, wherein the material comprises a gas permeability for oxygen of less than 0.05 cm³/cm² at 1 atm at 25° C. and gas permeability for carbon dioxide of at least 0.62 centimeters cubed per centimeters squared (cm³/cm²) at 1 atm at 25° C.

The present disclosure also provides for and includes method for treating a blood product comprising: adding an additive solution to the blood product; and storing the blood product in a DEHP-free blood compatible (BC) carbon dioxide permeable bag comprising a gas permeability for carbon dioxide of at least 0.62 cm³/cm² at about 1 atm at 25° C., wherein the storage is at least 7 days and the blood product comprises an oxygen level at the 7 days of storage that is decreased or about the same as an oxygen level in the blood product at day 1 of storage.

Further, the present disclosure provides for and includes a method for storing a storable blood comprising: placing a blood product in a storage container comprising: a DEHP-free blood compatible (BC) material having a permeability to carbon dioxide of at least 0.62 cm³/cm² at about 1 atm at 25° C. and a permeability to oxygen of no more than 0.3 cm³/cm² at about 1 atm, and a carbon dioxide sorbent; and storing the container comprising the storable blood for a period to prepare stored blood.

Also, the present disclosure provides for and includes a method for storing red blood cells comprising: placing the red blood cells in a storage container comprising: an outer oxygen and carbon dioxide impermeable container enclosing a DEHP-free blood compatible (BC) permeable inner collapsible container consisting of a material having a permeability to carbon dioxide of at least 0.62 cm³/cm² at about 1 atm and a permeability to oxygen of no more than 0.3 cm³/cm² at about 1 atm at 25° C. and enclosing a carbon dioxide sorbent, an oxygen sorbent, or an oxygen and a carbon dioxide sorbent between the inner and outer bag; and storing the container comprising the red blood cells for at least 7 days to prepare a stored blood product.

The present disclosure further provides for and includes a method for maintaining the level of 2,3-DPG in a blood product comprising: placing a blood product comprising an oxygen saturation of at least 10% in a storage container comprising an outer oxygen and carbon dioxide impermeable container enclosing a blood compatible (BC) material having a permeability to carbon dioxide of at least 0.62 cm³/cm² at about 1 atm at 25° C. and a permeability to oxygen of no more than 0.3 cm³/cm² at about 1 atm and enclosing a carbon dioxide sorbent between the inner and outer bag, and storing the container comprising the blood product, wherein the level of 2,3-DPG is increased for up to 14 days of storage compared to a level of 2,3-DPG of a blood product conventionally stored.

The present disclosure provides for and includes a method for maintaining the level of ATP a blood product comprising: placing a blood product comprising an oxygen saturation of at least 10% in a storage container comprising an outer oxygen and carbon dioxide impermeable container enclosing a blood compatible (BC) material having a permeability to carbon dioxide of at least 0.62 cm³/cm² at about 1 atm at 25° C. and a permeability to oxygen of no more than 0.3 cm³/cm² at about 1 atm and enclosing a carbon dioxide sorbent between the inner and outer bag; and storing the container comprising the blood product, wherein the level of ATP is increased after 42 days of storage compared to a level of ATP of a blood product conventionally stored.

The present disclosure further provides for and includes a composition comprising: a blood product selected from the group consisting of whole blood, platelets, and leukocytes; and an additive solution comprising sodium bicarbonate (NaHCO₃); sodium phosphate dibasic (Na₂HPO₄); adenine; guanosine; glucose; mannitol; N-acetyl-cysteine; 6-hydroxy-2,5,7,8-tetramethylchroman-2-carboxylic acid (Trolox); and 1-ascorbic acid (vitamin C).

The present disclosure further provides for and includes an additive composition comprising a concentration of: N-Acetyl-Cysteine; 6-Hydroxy-2,5,7,8-tetramethylchroman-2-carboxylic acid (Trolox); and 1-ascorbic acid, wherein the additive composition comprises a pH from 8 to 9.

The present disclosure further provides for and includes a composition comprising: a blood product selected from the group consisting of whole blood, platelets, and leukocytes; and an additive solution comprising a concentration of sodium phosphate dibasic (Na₂HPO₄); sodium citrate, adenine, guanosine, glucose, and mannitol.

### BRIEF DESCRIPTION OF THE DRAWINGS

Some aspects of the disclosure are herein described, by way of example only, with reference to the accompanying drawings. With specific reference now to the drawings in detail, it is stressed that the particulars shown are by way of example and are for purposes of illustrative discussion of embodiments of the disclosure. In this regard, the description, taken with the drawings, makes apparent to those skilled in the art how aspects of the disclosure may be practiced.

FIG. 1 is a schematic of an experimental setup according to an aspect of the present disclosure.

FIG. 2A is a graph showing the level of ATP at 21 days after storage in DEHP-free carbon dioxide permeable bags, with or without gas impermeable barrier bag. FIG. 2B is a graph showing ATP levels on day 42 after storage in DEHP-free carbon dioxide permeable bags, with or without gas impermeable barrier bag, on percent saturation oxygen of the red cell hemoglobin (%502), partial pressure of carbon dioxide (pCO₂), hemolysis, and ATP in an alkaline additive solution (AS7G-NAC) according to an aspect of the present disclosure. Data are the mean±SD of 10 independent tests (N=10).

FIGS. 3A and 3B are graphs showing the effects of storage of blood in DEHP-free carbon dioxide permeable bags, with or without gas impermeable barrier bag, on the levels of 2,3-DPG in red blood cells in an alkaline additive solution (AS7G-NAC) after 21 days (FIG. 3A) or 42 days (FIG. 3B) of storage, in an aspect of the present disclosure. Data are the mean±SD of 10 independent tests (N=10).

FIGS. 4A and 4B are graphs showing the effects of storage of blood in DEHP-free carbon dioxide permeable bags, with or without gas impermeable barrier bag, on the levels of % SO₂, pCO₂, hemolysis, and ATP in an AS3 additive solution according to an aspect of the present disclosure. FIG. 4A shows the ATP levels after 21 days and FIG. 4B shows the ATP levels after 42 days. Data are the mean±SD of 5 independent tests (N=5).

FIGS. 5A and 5B are graphs showing the effects of storage of blood in DEHP-free carbon dioxide permeable bags, with or without gas impermeable barrier bag, on the levels of % SO₂, pCO₂, hemolysis, and 2,3-DPG in an AS3 additive solution according to an aspect of the present disclosure. FIG. 5A presents the 2,3-DPG levels at 21 days, and FIG. 5B presents the 2,3-DPG levels at 42 days. Data are the mean±SD of 5 independent tests (N=5).

FIGS. 6A and 6B are graphs showing the effects of storage of blood in DEHP-free carbon dioxide permeable bags, with or without gas impermeable barrier bag, on the levels of % SO₂, pCO₂, hemolysis, and 2,3-DPG in AS7G-NAC (SOLX-NAC) additive solution according to an aspect of the present disclosure. FIG. 6A presents the 2,3-DPG levels at 21 days, and FIG. 6B presents the 2,3-DPG levels at 42 days. Data are the mean±SD of 3 independent tests (N=3)

FIGS. 7A and 7B are graphs showing the effects of storage of blood in DEHP-free carbon dioxide permeable bags, with or without gas impermeable barrier bag, on the levels of ATP in red blood cells in AS7G-NAC additive solution after 21 days or 42 days of storage, according to an aspect of the present disclosure. FIG. 7A presents the ATP levels at 21 days and FIG. 7B presents the ATP levels at 42 days. Data are the mean±SD of 3 independent tests (N=³).

Corresponding reference characters indicate corresponding parts throughout the several views. The examples set out herein illustrate several embodiments of the invention but should not be construed as limiting the scope of the invention in any manner.

### DETAILED DESCRIPTION

### Definitions

Unless defined otherwise, technical and scientific terms as used herein have the same meaning as commonly understood by one of ordinary skill in the art. One skilled in the art will recognize many methods can be used in the practice of the present disclosure. Indeed, the present disclosure is in no way limited to the methods and materials described. Any references cited herein are incorporated by reference in their entireties. For purposes of the present disclosure, the following terms are defined below.

As used herein the term “about” refers to ±10%.

The terms “comprises,” “comprising,” “includes,” “including,” “having,” and their conjugates mean “including but not limited to.”

The term “consisting of” means “including and limited to.”

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a,” “an,” and “the” include plural references unless the context clearly dictates otherwise. For example, the term “a compound” or “at least one compound” may include a plurality of compounds, including mixtures thereof.

Throughout this application, various embodiments of this disclosure may be presented in a range format. Accordingly, the description of a range should be considered to have specifically disclosed all the possible subranges as well as individual numerical values within that range. For example, description of a range such as “from 1 to 6” should be considered to have specifically disclosed subranges such as “from 1 to 3,” “from 1 to 4,” “from 1 to 5,” “from 2 to 4,” “from 2 to 6,” “from 3 to 6,” etc., as well as individual numbers within that range, for example, 1, 2, 3, 4, 5, and 6. Further, “from 1 to 3,” includes both 1 and 3. This applies regardless of the breadth of the range. As used herein, “between” means the range includes all the possible subranges as well as individual numerical values within that range but not including the external values. For example, “between 1 and 7” does not include the values 1 or 7 and between “0 and 7” does not include the values 0 or 7.

As used herein the term “method” refers to manners, means, techniques, and procedures for accomplishing a given task including, but not limited to, those manners, means, techniques, and procedures either known to or readily developed from known manners, means, techniques, and procedures by practitioners of the chemical, pharmacological, biological, biochemical, and medical arts.

As used herein, the term “bag” refers to collapsible containers prepared from a flexible material and includes pouches, tubes, and gusset bags. In certain aspects, a bag refers to non-collapsible container. As used herein, and included in the present disclosure, the term bag includes folded bags having one, two, three, or more folds and which are sealed or bonded on one, two, three, or more sides. Bags may be prepared using a variety of techniques known in the art including bonding of sheets of one or more materials. Methods of bonding materials to form bags are known in the art. See International Publication No. WO 2016/145210; Also included and provided for in the present disclosure are containers prepared by injection and blow molding. Methods to prepare blow molded and injection molded containers are known in the art. See U.S. Pat. Nos. 4,280,859; and 9,096,010. Preferred types of blow molded or injection molded containers are flexible containers that can be reduced in size for efficient packing and shipping while being capable of expanding to accommodate blood or blood components for reduction of oxygen. They also may be designed to conform to the volume of the blood until they are fully expanded. As used throughout the present disclosure, the bags are a form of collapsible container and the two terms are used interchangeably throughout the present disclosure.

As used herein the terms “blood” and “blood product” refers to whole blood, leukoreduced RBCs, platelet reduced RBCs, leukocyte and platelet reduced RBCs, platelets, and leukocytes. The term blood further includes packed red blood cells, platelet reduced packed red blood cells, leukocyte reduced packed red blood cells (LRpRBC), and leukocyte and platelet reduced packed red blood cells. The temperature of blood varies with the stage of the collection process, starting at the normal body temperature of 37° C. at the time and point of collection, but decreasing rapidly to about 30° C. once removed from the patient's body. Collected blood cools to room temperature in about 6 hours when untreated. In practice, the blood is processed within 24 hours and refrigerated at between about 2° C. and 6° C., usually 4° C.

As used herein, the term “whole blood” refers to a suspension of blood cells that contains red blood cells (RBCs), white blood cells (WBCs), platelets suspended in plasma, and includes electrolytes, hormones, vitamins, antibodies, etc. In certain aspects, whole blood is leukoreduced whole blood. In some aspects, whole blood is pathogen reduced or pathogen inactivated whole blood. In another aspect, whole blood is irradiated whole blood. In whole blood, white blood cells are normally present in the range between 4.5 and 11.0×10⁹ cells/L and the normal RBC range at sea level is 4.6-6.2×10¹²/L for men and 4.2-5.4×10¹²/L for women. The normal hematocrit, or percent packed cell volume, is about 40-54% for men and about 38-47% for women. The platelet count is normally 150-450×10⁹/L for both men and women. Whole blood is collected from a blood donor and is usually combined with an anticoagulant. Whole blood, when collected is initially at about 37° C. and rapidly cools to about 30° C. during and shortly after collection, but slowly cools to ambient temperature over about 6 hours. Whole blood may be processed according to methods of the present disclosure at collection, beginning at 30-37° C., or at room temperature (typically about 25° C.). As used herein, a “unit” of blood is about 450-500 ml including anticoagulant.

As used herein, a “blood donor” refers to a healthy individual from whom whole blood is collected, usually by phlebotomy or venipuncture, where the donated blood is processed and held in a blood bank for later use to be ultimately used by a recipient different from the donor. A blood donor may be selected based on biomarkers presented in the blood of the donor. A blood donor may be a subject scheduled for surgery or other treatment that may donate blood for themselves in a process known as autologous blood donation. Alternatively, and most commonly, blood is donated for use by another in a process known as heterologous transfusion. The collection of a whole blood sample drawn from a donor, or in the case of an autologous transfusion from a patient, may be accomplished by techniques known in the art, such as through donation or apheresis. Fresh, whole blood obtained from a donor using venipuncture has an oxygen saturation ranging from about 30% to about 88% saturated oxygen (SO₂) after addition of anticoagulant.

As used herein, “red blood cells” (RBCs) includes RBCs present in whole blood, leukoreduced RBCs, platelet reduced RBCs, and leukocyte and platelet reduced RBCs. Human red blood cells in vivo are in a dynamic state. The red blood cells contain hemoglobin, the iron-containing protein that carries oxygen throughout the body and gives red blood its color. The percentage of blood volume composed of red blood cells is called the hematocrit. As used herein, unless otherwise limited, RBCs also includes packed red blood cells (pRBCs). Packed red blood cells are prepared from whole blood using techniques commonly known in the art.

Platelets are small cellular components of blood that facilitate the clotting process by sticking to the lining of the blood vessels and facilitate healing by releasing growth factors when activated. The platelets, like the red blood cells, are made by the bone marrow and survive in the circulatory system for 9 to 10 days before they are removed by the spleen. Platelets are often prepared using a centrifuge to separate the platelets from the buffy coat sandwiched between the plasma layer and the pellet of red cells.

Storage of platelets has been extensively studied to identify the most favorable conditions including temperature, pH, O₂ and CO₂ concentrations. The result of this work was the conclusion that for stored platelets to persist in a recipient after transfusion, platelets require access to oxygen and storage at room temperature. Murphy and Gardner noted in 1975 that unwanted morphological changes were associated with reduced oxygen consumption. See, Murphy et al., “Platelet storage at 22 degrees C.: role of gas transport across plastic containers in maintenance of viability,” Blood 46(2):209-218 (1975). The authors observed that increased access to oxygen allows for aerobic metabolism (oxidative phosphorylation) resulting in a reduced rate of lactate production. At low P02 levels lactic acid production is increased consistent with the Pasteur effect. Moroff et al. noted that continuous oxygen consumption is required to maintain the pH of stored platelets at pH 7. See Moroff et al., “Factors Influencing Changes in pH during Storage of Platelet Concentrates at 20-24° C.,” Vox Sanguinis 42(1):33-45 (1982). Specially tailored container systems allow permeability to carbon dioxide as well as oxygen to prevent a lethal drop in pH. As shown by Kakaiya et al., “Platelet preservation in large containers,” Vox Sanguinis 46(2):111-118 (1984), maintaining platelet quality was the result of improved gas exchange conditions obtained with increased surface area available for gas exchange. The importance of maintaining oxygen levels during platelet storage led to the development of gas permeable containers and storage of platelets in oxygen enriched atmospheres. See U.S. Pat. No. 4,455,299, issued Jun. 19, 1984, to Grode. The importance of oxygen to the viability of stored platelets was reinforced by the observation that in an oxygen poor environment, the lactate levels increased 5 to 8-fold. See Kilkson et al., “Platelet metabolism during storage of platelet concentrates at 22 degrees C.,” Blood 64(2):406-14 (1984). Wallvik et al., “Platelet Concentrates Stored at 22° C. Need Oxygen The Significance of Plastics in Platelet Preservation,” Vox Sanguinis 45(4):303-311 (1983), reported that maintaining oxygen during the first five days of storage was critical for platelet preservation. Wallvik and co-workers also showed that the maximum platelet number that can be successfully stored for five days is predictable based on the determination of the oxygen diffusion capacity of the storage bag. See Wallvik et al, “The platelet storage capability of different plastic containers,” Vox Sanguinis 58(1):40-4 (1990). By providing blood bags with adequate gas exchange properties, pH is maintained, the loss of ATP and the release of alpha-granular platelet Factor 4 (PF4) was prevented. Each of the foregoing references are hereby incorporated in their entireties.

These findings, among others, led to practice standardization ensuring the oxygenation of platelets during room temperature storage to maximize post-transfusion viability. However, more recent studies have shown the effects of oxygen depletion on whole blood. For example, Yoshida, et al. discovered that cold storage enables anaerobic storage of platelets and provides known advantages of anaerobically stored RBCs observed in packed red blood cells, in the whole blood. See International Publication No. WO 2016/187353 at paragraph [0009]. “More specifically, while unexpectedly preserving the coagulability without introducing negative effects, deoxygenated whole blood provides for improved 2,3,-DPG levels.” See id.

Plasma is a protein-salt solution and the liquid portion of the blood in which red and white blood cells and platelets are suspended. Plasma is 90% water and constitutes about 55 percent of the blood volume. One function of plasma is to assist in blood clotting and immunity. Plasma is obtained by separating the liquid portion of the blood from the cells. Often, plasma is collected from the cells by centrifugation.

Reactive oxygen species (ROS) are produced by living organisms as a result of normal cellular metabolism. At high concentrations, and without the proper oxidant/antioxidant balance, ROS produce adverse modifications to cell components. Not to be limited by theory, it is believed that the antioxidants naturally occurring in the RBCs combined with the antioxidants in the storage solutions are adequate to reduce the effects of oxidative damage, for example to the RBC membrane, as long as additional oxygen is prevented from accumulating. In view of the initial antioxidant capacity, it is believed that much of the observed oxidative damage is not the result of the initial levels O₂, but rather accumulation and continued exposure. The results presented herein, show that the benefits of oxygen reduction can be achieved by preventing oxygen ingress into the cells during storage. This results in maintaining levels of oxygen well below the amount needed for saturation of the naturally occurring antioxidant. Even further, under conditions of high carbon dioxide permeability with alkaline additive solutions, high levels of 2,3-DPG can be maintained even at high oxygen saturation levels. In contrast, oxygen and carbon dioxide levels during storage under conventional methods increase throughout the storage period and lower ATP and 2,3-DPG levels are observed. By preventing oxygen ingress or preventing oxygen ingress and maintaining the initial low levels of oxygen in the blood during processing requirements can be reduced such that the oxygen present during storage does not overwhelm the antioxidant capacity of the cells. The results presented below demonstrate that reducing the level of CO₂ during storage results in increased concentrations of 2,3-DPG, and increased levels of 2,3-DPG and ATP when combined with an outer barrier and sorbent to manage oxygen during the storage period.

To achieve this result, the present disclosure provides for, and includes a method for the storage of a blood product comprising: obtaining an oxygenated blood product having a % SO₂ of greater than 30%; adding an additive solution to said blood product; and storing the blood product in a di-2-ethylhexyl phthalate free (DEHP-free) blood compatible (BC) carbon dioxide permeable bag comprising a gas permeability for carbon dioxide of at least 0.62 centimeters cubed per centimeters squared (cm³/cm²) at about 1 atm at 25° C. In an aspect, the DEHP-free blood compatible (BC) carbon dioxide permeable bag further comprises butyryltrihexylcitrate (BTHC). In another aspect, the DEHP-free blood compatible (BC) carbon dioxide permeable bag further comprises 1,2-Cyclohexane dicarboxylic acid diisononyl ester (DINCH).

### Methods

Storage in CO2 Permeable Bags without Oxygen Control

In an aspect of the present disclosure, the methods provide for a blood product that is stored in said (DEHP-free) blood compatible (BC) carbon dioxide permeable bag for at least 7 days. In aspects, the 2,3-DPG level is increased above the levels in conventionally stored blood. In another aspect, the methods provide for a blood product that is stored for at least 14 days. In yet another aspect, the methods provide for a blood product that is stored for at least 21 days. In another aspect, the methods provide for a blood product that is stored for at least 28 days. In yet another aspect, the methods provide for a blood product that is stored for at least 35 days. In a further aspect, the blood product is stored for at least 40 days. The present methods further provide for blood products that are stored for 56 days. Notably, this is the first report of storage conditions that provide for transfusable quality blood at 56 days. In another aspect, a blood product is stored for up to 7, 14, 21, 35, 42, or 56 days. In yet another aspect, a blood product is stored between 1 and 7, 1 and 14, 1 and 21, 1 and 35, 1 and 42, 1 and 56, 7 and 14, 7 and 21, 7 and 35, 7 and 42, 7 and 56, 14 and 21, 14 and 28, 14 and 35, 14 and 42, 14 and 56, 21 and 35, 21 and 42, 35 and 42 days, or 35 and 56 days.

The methods of the present disclosure provide for the storage of venous collected blood products that are not processed to reduce oxygen and have an initial % SO₂ ranging between 30 and 100% before storage in DEHP-free) blood compatible (BC) carbon dioxide permeable bag. In an aspect of the present disclosure, the methods provide for the storage of a venous collected blood product that has a % SO₂ of greater than 40% at the beginning of the storage period (e.g., day zero). In another aspect, the methods provide for the storage of an oxygenated blood product that has a % SO₂ of greater than 50%. In another aspect, the methods provide for the storage of oxygenated blood products having a % SO₂ of greater than 60%. In another aspect, an oxygenated blood product has a % SO₂ of greater than 70%. In another aspect, an oxygenated blood product has a % SO₂ of greater than 80%. In another aspect, an oxygenated blood product has a % SO₂ of greater than 90%. In another aspect, an oxygenated blood product has a % SO₂ of between 30 and 80%. In another aspect, an oxygenated blood product has a % SO₂ of between 50 and 90%. In another aspect, an oxygenated blood product has a % SO₂ of between 40 and 100%. In another aspect, an oxygenated blood product has a % SO₂ of at least 30%. In another aspect, an oxygenated blood product has a % SO₂ of at least 50%.

The present disclosure provides for, and includes a method for the storage of a blood product comprising: obtaining a venous collected blood product having a % SO₂ of greater than 30%; adding an additive solution to said blood product; and storing the blood product in a DEHP-free blood compatible (BC) carbon dioxide permeable bag comprising a gas permeability for carbon dioxide of at least 0.62 centimeters cubed per centimeters squared (cm³/cm²) at about 1 atm at 25° C. In an aspect, the DEHP-free blood compatible (BC) carbon dioxide permeable bag is a PVC bag further comprising BTHC. In an aspect, the DEHP-free BC carbon dioxide permeable bag is a PVC bag further comprising DINCH. In yet another aspect, the DEHP-free blood compatible (BC) carbon dioxide permeable bag further comprises EXP500.

In an aspect of the present disclosure, a stored blood product has a pCO₂ of less than 125 mmHg during initial blood collection. In another aspect, the blood product has a pCO₂ of less than 100 mmHg. In another aspect, the blood product has a pCO₂ of less than 75 mmHg. In another aspect, the blood product has a pCO₂ of less than 50 mmHg. In another aspect, the blood product has a pCO₂ of less than 25 mmHg. In another aspect, the blood product has a pCO₂ of between 125 and 100 mmHg. In another aspect, the blood product has a pCO₂ of between 100 and 75 mmHg. In another aspect, the blood product has a pCO₂ of between 75 and 25 mmHg. In aspects of the methods, the 2,3-DPG level in the stored blood product is increased by at least 10% compared to 2,3-DPG levels of a conventionally stored blood product. In other aspects, ATP levels in the stored blood product are increased by at least 10% in said storable blood product during said storing compared to ATP levels of a conventionally stored blood product. In yet other aspects, 2,3-DPG level in the stored blood product is increased by at least 10% and ATP levels are increased by at least 10% compared to 2,3-DPG and ATP levels of a conventionally stored blood product. In aspects of the methods, the 2,3-DPG level in the stored blood product is increased by at least 15% compared to 2,3-DPG levels of a conventionally stored blood product. In yet other aspects, 2,3-DPG level is increased by at least 10% and ATP levels are increased by at least 15% compared to 2,3-DPG and ATP levels of a conventionally stored blood product.

In an aspect of the present disclosure, wherein said method further comprises depleting CO2 during the storage period to a level of between 125 mmHg and 25 mmHg after a storage period of up to 56 days to produce a CO2 reduced stored blood product. In an aspect the stored blood product has a pCO₂ of less than 125 mmHg and a % SO₂ of greater than 20% after storage for at least 7 days. In another aspect, the blood product has a pCO₂ of less than 100 mmHg and a % SO₂ of greater than 20%. In another aspect, the blood product has a pCO₂ of less than 75 mmHg and a % SO₂ of greater than 20%. In another aspect, the blood product has a pCO₂ of less than 50 mmHg and a % SO₂ of greater than 20%. In another aspect, on Day 56 of storage, the blood product has a pCO₂ of less than 25 mmHg and a % SO₂ of greater than 20%. In another aspect, on Day 56 of storage, the blood product has between 125 mmHg and 25 mmHg, and a % SO₂ of greater than 20%. In another aspect, on Day 56 of storage, the blood product has between 125 mmHg and 25 mmHg, and a % SO₂ of greater than 5%. In another aspect, on Day 56 of storage, the blood product has between 125 mmHg and 25 mmHg, and a % SO₂ of between 3 and 20%. In yet another aspect, the blood product has a pCO₂ of less than 125 mmHg and a % SO₂ of greater than 15%. In another aspect, the blood product has a pCO₂ of less than 100 mmHg and a % SO₂ of greater than 15%. In another aspect, the blood product has a pCO₂ of less than 75 mmHg and a % SO₂ of greater than 15%. In another aspect, the blood product has a pCO₂ of less than 50 mmHg and a % SO₂ of greater than 15%. In another aspect, the blood product has a pCO₂ of less than 25 mmHg and a % SO₂ of greater than 15%. In a further aspect, the blood product has a pCO₂ of less than 125 mmHg and a % SO₂ of greater than 10%. In another aspect, the blood product has a pCO₂ of less than 100 mmHg and a % SO₂ of greater than 10%. In another aspect, the blood product has a pCO₂ of less than 75 mmHg and a % SO₂ of greater than 10%. In another aspect, the blood product has a pCO₂ of less than 50 mmHg and a % SO₂ of greater than 10%. In another aspect, the blood product has a pCO₂ of greater than 25 mmHg and a % SO₂ of greater than 10%. In another aspect, the blood product has a pCO₂ of less than 125 mmHg and a % SO₂ of between 5 and 30%. In another aspect, the blood product has a pCO₂ of less than 100 mmHg and a % SO₂ of between 5 and 30%. In another aspect, the blood product has a pCO₂ of less than 75 mmHg and a % SO₂ of between 5 and 30%. In another aspect, the blood product has a pCO₂ of less than 50 mmHg and a % SO₂ of between 5 and 30%. In another aspect, the blood product has a pCO₂ of less than 25 mmHg and a % SO₂ of between 5 and 30%.

In another aspect, the methods provide for depleting CO2 during the storage period to a level of between 125 mmHg and 25 mmHg after a storage period of 21 days to produce a CO2 reduced stored blood product having a % SO₂ of greater than 20%, and having increased 2,3-DPG levels compared to a conventionally stored blood product. In an aspect, the methods provide for depleting CO2 during the storage period to a level of between 125 mmHg and 25 mmHg after a storage period of 21 days to produce a CO2 reduced stored blood product having a % SO₂ of greater than 20%, and having increased 2,3-DPG and ATP levels compared to a conventionally stored blood product. In an aspect, a stored blood product has a pCO₂ of less than 125 mmHg and a % SO₂ of greater than 20%. In another aspect, the blood product has a pCO₂ of less than 100 mmHg and a % SO₂ of greater than 20%. In another aspect, on day 21 of storage, the blood product has a pCO₂ of less than 75 mmHg and a % SO₂ of greater than 20%. In another aspect, the blood product has a pCO₂ of less than 50 mmHg and a % SO₂ of greater than 20%. In another aspect, the blood product has a pCO₂ of less than 25 mmHg and a % SO₂ of greater than 20%. In yet another aspect, the blood product having a pCO₂ of less than 125 mmHg and a % SO₂ of greater than 15%. In another aspect, the blood product has a pCO₂ of less than 100 mmHg and a % SO₂ of greater than 15%. In another aspect, the blood product has a pCO₂ of less than 75 mmHg and a % SO₂ of greater than 15%. In another aspect, the blood product has a pCO₂ of less than 50 mmHg and a % SO₂ of greater than 15%. In another aspect, the blood product has a pCO₂ of less than 25 mmHg and a % SO₂ of greater than 15%. In a further aspect, the blood product has a pCO₂ of less than 125 mmHg and a % SO₂ of greater than 10%. In another aspect, the blood product has a pCO₂ of less than 100 mmHg and a % SO₂ of greater than 10%. In another aspect, the blood product has a pCO₂ of less than 75 mmHg and a % SO₂ of greater than 10%. In another aspect, the blood product has a pCO₂ of less than 50 mmHg and a % SO₂ of greater than 10%. In another aspect, the blood product has a pCO₂ of less than 25 mmHg and a % SO₂ of greater than 10%. In aspects of the methods, the 2,3-DPG level is increased by at least 10% compared to 2,3-DPG levels of a conventionally stored blood product. In other aspects, ATP levels are increased by at least 10% in said storable blood product during said storing compared to ATP levels of a conventionally stored blood product. In yet other aspects, 2,3-DPG level is increased by at least 10% and ATP levels are increased by at least 10% compared to 2,3-DPG and ATP levels of a conventionally stored blood product. In aspects of the methods, the 2,3-DPG level is increased by at least 15% compared to 2,3-DPG levels of a conventionally stored blood product. In yet other aspects, 2,3-DPG level is increased by at least 10% and ATP levels are increased by at least 15% compared to 2,3-DPG and ATP levels of a conventionally stored blood product.

### Storage in CO2 Permeable Bags Oxygen Control

The present disclosure provides for, and includes a method for the storage of a blood product comprising: obtaining an oxygenated blood product having a % SO₂ of greater than 30%; adding an additive solution to said blood product; and storing the blood product in a di-2-ethylhexyl phthalate free (DEHP-free) blood compatible (BC) carbon dioxide permeable bag comprising a gas permeability for carbon dioxide of at least 0.62 centimeters cubed per centimeters squared (cm³/cm²) at about 1 atm at 25° C., and further comprising an oxygen impermeable barrier bag and sorbent to prevent the introgression of oxygen and saturation of the blood. In an aspect, the DEHP-free blood compatible (BC) carbon dioxide permeable bag further comprises BTHC. In another aspect, the DEHP-free blood compatible (BC) carbon dioxide permeable bag further comprises DINCH.

In an aspect, the method further comprises an oxygen impermeable barrier bag to prevent the introgression of oxygen and saturation of the blood. In an aspect, the DEHP-free blood compatible (BC) carbon dioxide permeable bag further comprises BTHC. In another aspect, the DEHP-free blood compatible (BC) carbon dioxide permeable bag further comprises DINCH. In yet another aspect methods of the present disclosure provide for treating a blood product comprising adding an additive solution to the blood product, and storing the blood product in a blood compatible (BC) carbon dioxide permeable bag comprising a gas permeability for carbon dioxide of at least 0.62 cm³/cm² at about 1 atm at 25° C., wherein said storage bag further comprises an outer bag impermeable to oxygen and carbon dioxide and the outer bag encloses a carbon dioxide and oxygen sorbent placed between said BC carbon dioxide permeable bag and said outer bag. In aspects of the method the storage is at least 7 days and the blood product comprises an oxygen level of between 5 and 30% at 7 days of storage that is decreased or about the same as an oxygen level in the blood product at day 1 of storage. In another aspect, the blood product comprises an oxygen level of between 5 and 30% at 14 days of storage that is decreased or about the same as the oxygen level in the blood product at day 1 of storage. In another aspect, the blood product comprises an oxygen level of between 5 and 30% at 21 days of storage that is decreased or about the same as the oxygen level in the blood product at day 1 of storage. In another aspect, the blood product comprises an oxygen level of between 5 and 30% at 28 days of storage that is decreased or about the same as the oxygen level in the blood product at day 1 of storage. In another aspect, the blood product comprises an oxygen level of between 5 and 30% at 32 days of storage that is decreased or about the same as the oxygen level in the blood product at day 1 of storage. In another aspect, the blood product comprises an oxygen level of between 5 and 30% at 38 days of storage that is decreased or about the same as the oxygen level in the blood product at day 1 of storage. In another aspect, the blood product comprises an oxygen level of between 5 and 30% at 42 days of storage that is decreased or about the same as the oxygen level in the blood product at day 1 of storage.

In yet another aspect methods of the present disclosure provide for treating a blood product comprising adding an additive solution to the blood product, and storing the blood product in a blood compatible (BC) carbon dioxide permeable bag comprising a gas permeability for carbon dioxide of at least 0.62 cm³/cm² at about 1 atm at 25° C., wherein said storage bag further comprises an outer bag impermeable to oxygen and carbon dioxide and the outer bag encloses a carbon dioxide and oxygen sorbent placed between said BC carbon dioxide permeable bag and said outer bag. In aspects of the method, the storage is at least 7 days and the blood product comprises an oxygen level of greater than 30% at 7 days of storage that is decreased or about the same as an oxygen level in the blood product at day 1 of storage. In another aspect, the blood product comprises an oxygen level of greater than 30% at 14 days of storage that is decreased or about the same as the oxygen level in the blood product at day 1 of storage. In another aspect, the blood product comprises an oxygen level of greater than 30% at 21 days of storage that is decreased or about the same as the oxygen level in the blood product at day 1 of storage. In another aspect, the blood product comprises an oxygen level of greater than 30% at 28 days of storage that is decreased or about the same as the oxygen level in the blood product at day 1 of storage. In another aspect, the blood product comprises an oxygen level of greater than 30% at 32 days of storage that is decreased or about the same as the oxygen level in the blood product at day 1 of storage. In another aspect, the blood product comprises an oxygen level of greater than 30% at 38 days of storage that is decreased or about the same as the oxygen level in the blood product at day 1 of storage. In another aspect, the blood product comprises an oxygen level of greater than 30% at 42 days of storage that is decreased or about the same as the oxygen level in the blood product at day 1 of storage. In aspects of the method, the 2,3-DPG level is increased by at least 10% compared to 2,3-DPG levels of a conventionally stored blood product. In other aspects, ATP levels are increased by at least 10% in said storable blood product during said storing compared to ATP levels of a conventionally stored blood product. In yet other aspects, 2,3-DPG level is increased by at least 10% and ATP levels are increased by at least 10% compared to 2,3-DPG and ATP levels of a conventionally stored blood product. In aspects of the methods, the 2,3-DPG level is increased by at least 15% compared to 2,3-DPG levels of a conventionally stored blood product. In yet other aspects, 2,3-DPG level is increased by at least 10% and ATP levels are increased by at least 15% compared to 2,3-DPG and ATP levels of a conventionally stored blood product.

The present disclosure provides for, and includes, a method for maintaining the level of 2,3-DPG in a blood product comprising: placing a non-deoxygenated blood product in a storage container comprising a blood compatible (BC) material having a permeability to carbon dioxide of at least 0.62 cm³/cm² at about 1 atm at 25° C. and a permeability to oxygen of no more than 0.3 cm³/cm² at about 1 atm, and the storage bag is enclosed in an outer bag impermeable to oxygen and carbon dioxide that further encloses a carbon dioxide sorbent, wherein the blood product comprises an initial oxygen saturation of at least 10%; and storing the container comprising the blood product, wherein the level of 2,3-DPG is increased at 14 days of storage compared to a level of 2,3-DPG of a blood product conventionally stored. In another aspect, the 2,3-DPG level is increased at 21 days of storage compared to a level of 2,3-DPG of a blood product conventionally stored. In another aspect, the 2,3-DPG level is increased at 28 days of storage compared to a level of 2,3-DPG of a blood product conventionally stored. In another aspect, the 2,3-DPG level is increased at 35 days of storage compared to a level of 2,3-DPG of a blood product conventionally stored. In another aspect, the 2,3-DPG level is increased at 42 days of storage compared to a level of 2,3-DPG of a blood product conventionally stored. In an aspect, the DEHP-free blood compatible (BC) carbon dioxide permeable bag is a PVC bag further comprising BTHC. In an aspect, the DEHP-free BC carbon dioxide permeable bag is a PVC bag further comprising DINCH. In yet another aspect, the DEHP-free blood compatible (BC) carbon dioxide permeable bag further comprises EXP500.

In another aspect, the method for maintaining the level of 2,3-DPG in a blood product provides for increased 2,3-DPG levels of between 10% and 70% compared to conventionally stored blood. In an aspect, the method provides for 2,3-DPG levels that are increased by at least 10%, 15%, 20%, 25%, 30%, 35%, 40%, 45%, 50%, 60%, 70%, or more compared to a level of 2,3-DPG of a blood product conventionally stored. In certain aspects, the 2,3-DPG level is increased by at least 10% at 7 days of storage compared to a level of 2,3-DPG of a blood product conventionally stored. In another aspect, the 2,3-DPG level is increased by at least 10% at 14 days of storage compared to a level of 2,3-DPG of a blood product conventionally stored. In another aspects, the 2,3-DPG level is increased by at least 10% at 21 days of storage compared to a level of 2,3-DPG of a blood product conventionally stored. In certain aspects, the 2,3-DPG level is increased by at least 10% at 28 days of storage compared to a level of 2,3-DPG of a blood product conventionally stored. In yet another aspect, the 2,3-DPG level is increased by at least 10% at 42 days of storage compared to a level of 2,3-DPG of a blood product conventionally stored. In certain aspects, the 2,3-DPG level is increased by at least 20% at 7 days of storage compared to a level of 2,3-DPG of a blood product conventionally stored. In another aspect, the 2,3-DPG level is increased by at least 20% at 14 days of storage compared to a level of 2,3-DPG of a blood product conventionally stored. In another aspects, the 2,3-DPG level is increased by at least 20% at 21 days of storage compared to a level of 2,3-DPG of a blood product conventionally stored. In certain aspects, the 2,3-DPG level is increased by at least 20% at 28 days of storage compared to a level of 2,3-DPG of a blood product conventionally stored. In yet another aspect, the 2,3-DPG level is increased by at least 20% at 42 days of storage compared to a level of 2,3-DPG of a blood product conventionally stored. In certain aspects, the 2,3-DPG level is increased by at least 30% at 7 days of storage compared to a level of 2,3-DPG of a blood product conventionally stored. In another aspect, the 2,3-DPG level is increased by at least 30% at 14 days of storage compared to a level of 2,3-DPG of a blood product conventionally stored. In another aspects, the 2,3-DPG level is increased by at least 30% at 21 days of storage compared to a level of 2,3-DPG of a blood product conventionally stored. In certain aspects, the 2,3-DPG level is increased by at least 30% at 28 days of storage compared to a level of 2,3-DPG of a blood product conventionally stored. In yet another aspect, the 2,3-DPG level is increased by at least 30% at 42 days of storage compared to a level of 2,3-DPG of a blood product conventionally stored. In a further aspect, the 2,3-DPG level is increased by at least 40% at 28 days of storage compared to a level of 2,3-DPG of a blood product conventionally stored. In yet another aspect, the 2,3-DPG level is increased by at least 40% at 42 days of storage compared to a level of 2,3-DPG of a blood product conventionally stored. In another aspect, the 2,3-DPG level is increased by at least 50% at 28 days of storage compared to a level of 2,3-DPG of a blood product conventionally stored. In yet another aspect, the 2,3-DPG level is increased by at least 50% at 42 days of storage compared to a level of 2,3-DPG of a blood product conventionally stored. In another aspect, the 2,3-DPG level is increased by at least 60% at 42 days of storage compared to a level of 2,3-DPG of a blood product conventionally stored. In another aspect, the 2,3-DPG level is increased by at least 70% at 42 days of storage compared to a level of 2,3-DPG of a blood product conventionally stored. In yet another aspect, the 2,3-DPG level is increased by at least 80% at 42 days of storage compared to a level of 2,3-DPG of a blood product conventionally stored. In a further aspect, the 2,3-DPG level is increased by at least 90% at 42 days of storage compared to a level of 2,3-DPG of a blood product conventionally stored. In yet another aspect, the 2,3-DPG level is increased by between 50 and 90% at 42 days of storage compared to a level of 2,3-DPG of a blood product conventionally stored.

The present disclosure further provides for, and includes, a method for maintaining the level of ATP a blood product comprising: placing an oxygenated blood product in a storage container comprising a blood compatible (BC) material having a permeability to carbon dioxide of at least 0.62 cm³/cm² at about 1 atm at 25° C. and a permeability to oxygen of no more than 0.3 cm³/cm² at about 1 atm, and the storage bag is enclosed in an outer bag impermeable to oxygen and carbon dioxide that further encloses a carbon dioxide and oxygen sorbent, wherein said blood product has an oxygen saturation of at least 10%; and storing the container comprising said blood product, wherein the level of ATP is increased after 7 days of storage compared to a level of ATP of a blood product conventionally stored. In another aspect, the ATP level is increased at 14 days of storage compared to a level of ATP of a blood product conventionally stored. In another aspect, the ATP level is increased at 21 days of storage compared to a level of ATP of a blood product conventionally stored. In another aspect, the ATP level is increased for up to 28 days of storage compared to a level of ATP of a blood product conventionally stored. In another aspect, the ATP level is increased for up to 35 days of storage compared to a level of ATP of a blood product conventionally stored. In another aspect, the ATP level is increased for up to 42 days of storage compared to a level of ATP of a blood product conventionally stored. In another aspect, the ATP level is increased by at least 10%, 15%, 20%, 25%, 30%, 35%, 40%, 45%, 50%, 60%, 70%, or more compared to a level of ATP of a blood product conventionally stored. In certain aspects, the ATP level is increased by at least 10% at 7 days of storage compared to a level of ATP of a blood product conventionally stored. In another aspect, the ATP level is increased by at least 10% at 14 days of storage compared to a level of ATP of a blood product conventionally stored. In another aspects, the ATP level is increased by at least 10% at 21 days of storage compared to a level of ATP of a blood product conventionally stored. In certain aspects, the ATP level is increased by at least 10% at 28 days of storage compared to a level of ATP of a blood product conventionally stored. In yet another aspect, the ATP level is increased by at least 10% at 42 days of storage compared to a level of ATP of a blood product conventionally stored. In certain aspects, the ATP level is increased by at least 20% at 7 days of storage compared to a level of ATP of a blood product conventionally stored. In another aspect, the ATP level is increased by at least 20% at 14 days of storage compared to a level of ATP of a blood product conventionally stored. In another aspects, the ATP level is increased by at least 20% at 21 days of storage compared to a level of ATP of a blood product conventionally stored. In certain aspects, the ATP level is increased by at least 20% at 28 days of storage compared to a level of ATP of a blood product conventionally stored. In yet another aspect, the ATP level is increased by at least 20% at 42 days of storage compared to a level of ATP of a blood product conventionally stored. In certain aspects, the ATP level is increased by at least 30% at 7 days of storage compared to a level of ATP of a blood product conventionally stored. In another aspect, the ATP level is increased by at least 30% at 14 days of storage compared to a level of ATP of a blood product conventionally stored. In another aspects, the ATP level is increased by at least 30% at 21 days of storage compared to a level of ATP of a blood product conventionally stored. In certain aspects, the ATP level is increased by at least 30% at 28 days of storage compared to a level of ATP of a blood product conventionally stored. In yet another aspect, the ATP level is increased by at least 30% at 42 days of storage compared to a level of ATP of a blood product conventionally stored. In a further aspect, the ATP level is increased by at least 40% at 28 days of storage compared to a level of ATP of a blood product conventionally stored. In yet another aspect, the ATP level is increased by at least 40% at 42 days of storage compared to a level of ATP of a blood product conventionally stored. In another aspect, the ATP level is increased by at least 50% at 28 days of storage compared to a level of ATP of a blood product conventionally stored. In yet another aspect, the ATP level is increased by at least 50% at 42 days of storage compared to a level of ATP of a blood product conventionally stored. In an aspect, the DEHP-free blood compatible (BC) carbon dioxide permeable bag is a PVC bag further comprising BTHC. In an aspect, the DEHP-free BC carbon dioxide permeable bag is a PVC bag further comprising DINCH. In yet another aspect, the DEHP-free blood compatible (BC) carbon dioxide permeable bag further comprises EXP500.

The present disclosure further provides for, and includes, a method for maintaining the level of hemolysis in a blood product below 0.8% after 7 days of storage in the absence of DEHP. In another aspect, the level of hemolysis in a blood product is maintained below 0.8% after 14 days of storage. In another aspect, the level of hemolysis in a blood product is maintained below 0.8% after 21 days of storage. In another aspect, the level of hemolysis in a blood product is maintained below 0.8% after 28 days of storage. In another aspect, the level of hemolysis in a blood product is maintained below 0.8% after 35 days of storage. In another aspect, the level of hemolysis in a blood product is maintained below 0.8% after 42 days of storage.

### DEHP and Other Plasticizers

The use of PVC in the manufacture of collapsible blood containers is well known in the art. The use of various plasticizers in various PVC formulations is also well known in the art, in particular diethylhexyl phthalate (DEHP) has been universally adopted for the long-term storage of red blood cells. In addition to increasing the flexibility of the PVC, DEHP also increases the permeability of PVC to oxygen. For these reasons, DEHP has been used as a plasticizer for storing red blood cells. An exemplary PVC-DEHP film is the Renolit ES-3000 film (American Renolit Corp., City of Commerce, Calif.).

DEHP improves the storability of red blood cells however recently, concerns have been raised for the safety of DEHP. RBC compositions which are stored in PVC-DEHP bags extract DEHP from the bag. Studies have shown that by day 28 of storage, RBCs stored in PVC-DEHP have approximately 80 mg/ml of DEHP. Rock et al., “Distribution of di(2-ethylhexyl) phthalate and products in blood and blood components,” Environ Health Perspect. 65:309-316 (1986). While still controversial and under debate, some reports have suggested that DEHP can interfere with normal hormone function and has been linked to asthma, breast cancer, obesity and type 2 diabetes, brain development issues, attention deficit hyperactivity disorder (ADHD), autism spectrum disorders, and lowered male fertility. In other studies, DEHP has been shown to induce the formation of stomatocytes and increase the exposure of phosphatidylserine in a suspension of red blood cells. See Melzak et al., “The Blood Bag Plasticizer Di-2-Ethylhexylphthalate Causes Red Blood Cells to Form Stomatocytes, Possibly by Inducing Lipid Flip-Flop” Transfus Med Hemother. 45(6): 413-422 (2018). For this reason, Europe is considering the adoption of measures to protect people from DEHP exposure.

In the course of studies examining DEHP-free materials suitable for blood storage, results reveal for the first time the role of CO₂ depletion alone in maintaining high levels of key metabolites (e.g., 2,3-DPG and ATP) during storage. Prior to the results provided below, control of CO2 during storage was largely limited to preventing changes to pH and reaction with CO2 sensitive reagents. For example, U.S. Pat. No. 4,228,032, issued Apr. 4, 1978, to Talcott, taught CO₂ absorption during storage of bicarbonate containing buffers such as BAGPAM to maintain an alkaline storage environment. Talcott shows CO₂ absorption during storage by silicone rubber compounded with Ca(OH)₂ maintains alkaline pH. However, Talcott does not teach or suggest any specific effects of CO₂ on the storage of blood or suggest that blood metabolite levels are affected by CO2 during storage. Instead, Talcott teaches maintaining an alkaline storage environment to maintain the levels of 2,3-DPG. More recently, a role for carbon dioxide during storage of oxygen depleted pRBCs suggested a role for CO2 on 2,3-DPG levels. See International Patent Publication No. WO 2012/027582, published Mar. 1, 2012 (the “'582 PCT”). The '582 PCT showed that removal pre-storage depletion of oxygen to about 10 mmHg and carbon dioxide to 5 mmHg prior to storage can improve 2,3-DPG and ATP levels relative to conventionally stored blood. The '582 PCT also showed that the effect on 2,3-DPG was largely due to an effect of carbon dioxide depletion. The present disclosure is the first showing that depletion of CO₂ and maintenance of oxygen levels during storage can maintain 2,3-DPG and ATP levels during storage. Prior to the present disclosure, studies have shown the importance of pH and oxygen depletion prior to storage, not the importance of depleting CO₂ alone (while maintaining oxygen) and during storage for the maintenance of key metabolites, including 2,3-DPG and ATP. The unexpected finding that management of gas exchange during storage can achieve similar results to deplete and store methods greatly simplifies the preparation of blood for storage and transfusion. Moreover, the present results demonstrate that the CO₂ effect can be achieved at CO₂ levels more than 10 times higher than tested in the '582 PCT.

The present disclosure provides for various materials and plasticizers that can be used instead of DEHP. The present disclosure provides for suitable materials with increased carbon dioxide permeability.

The present disclosure provides for suitable PVC materials for use in a collapsible blood container that is substantially permeable to carbon dioxide. The use of a PVC-citrate film such as Renolit ES-4000 (American Renolit Corp., City of Commerce, Calif.) having a thickness of from about 5 μm to about 250 μm, and more preferably from about 10 μm to about 100 μm is suitable for providing a collapsible blood container having the desired characteristics of high carbon dioxide permeability, Radio Frequency (RF) welding and joining, and high tensile strength. RF welding is also known in the art as high frequency welding or dielectric welding. RF welding is a method of joining thin sheets of material or film together using high frequency electromagnetic energy to fuse the materials. In aspects of the present disclosure, RF welding is used to fuse films together to avoid gas leakage or ingress while forming a collapsible blood container.

In certain aspects, carbon dioxide permeable membranes suitable for use in the preparation of a collapsible blood container comprise PVC without the plasticizer di-2-ethylhexyl phthalate (DEHP). In another aspect, carbon dioxide permeable membranes suitable for use in the preparation of a collapsible blood container comprise PVC without di-2-ethylhexyl terephthalate (DEHT). In another aspect, carbon dioxide permeable membranes suitable for use in the preparation of a collapsible blood container comprise PVC with 1,2-Cyclohexane dicarboxylic acid diisononyl ester (DINCH). In another aspect, carbon dioxide permeable membranes suitable for use in the preparation of a collapsible blood container comprise PVC with butyryltrihexylcitrate (BTHC) In certain aspects, the concentration of the plasticizers is between 20 and 70% weight/weight in PVC. In another aspects, the concentration of the plasticizers is between 20 and 40% weight/weight in PVC. In another aspect, the concentration of the plasticizers is between 40 and 70% weight/weight in PVC. In another aspect, the concentration of the plasticizers is greater than 20% weight/weight in PVC. In another aspect, the concentration of the plasticizers is greater than 30% weight/weight in PVC. In another aspect, the concentration of the plasticizers is greater than 40% weight/weight in PVC. In another aspect, the concentration of the plasticizers is greater than 50% weight/weight in PVC. In another aspect, the concentration of the plasticizers is greater than 60% weight/weight in PVC. In certain aspects, the plasticizer DINCH is more preferably between 20-45% weight/weight in PVC. In certain aspects, the plasticizer DINCH is greater than 20% weight/weight in PVC. In certain aspects, the plasticizer DINCH is greater than 30% weight/weight in PVC. In certain aspects, the plasticizer DINCH is greater than 40% weight/weight in PVC.

In another aspect, carbon dioxide permeable membranes suitable for use in the preparation of a collapsible blood container comprise polyolefin. In a further aspect, carbon dioxide permeable membranes suitable for use in the preparation of a collapsible blood container comprise silicone. In another aspect, carbon dioxide permeable membranes suitable for use in the preparation of a collapsible blood container comprise polyvinylidene fluoride (PVDF) however these membranes are not strong enough for storage and further result in increased hemolysis. In another aspect, carbon dioxide permeable membranes suitable for use in the preparation of a collapsible blood container comprise polysulphone (PS), though like PVDF, it exhibits increased hemolysis and brittleness. In another aspect, carbon dioxide permeable membranes suitable for use in the preparation of a collapsible blood container comprise polypropylene (PP). In another aspect, carbon dioxide permeable membranes suitable for use in the preparation of a collapsible blood container comprise polyurethane.

### Inner Bag Materials and Permeability

The present disclosure provides for and includes blood storage containers that provide for depleting carbon dioxide from blood during storage comprising a carbon dioxide permeable bag that is permeable to carbon dioxide and impermeable to oxygen. Preferably, the blood storage containers are prepared from DEHP-free carbon dioxide permeable materials.

The present disclosure provides for, and includes, DEHP-free carbon dioxide permeable bags prepared from membranes that are characterized primarily by their permeability to carbon dioxide.

The present disclosure also provides for, and includes, membranes that are permeable to carbon dioxide. Membranes that are permeable to carbon dioxide are used in the present disclosure for the preparation of carbon dioxide permeable bags, preferably DEHP-free carbon dioxide permeable membranes. In certain aspects, the membranes that are permeable to carbon dioxide are also biocompatible membranes, approved and suitable for extended contact with blood that is to be transfused into a patient. Like substantially impermeable membranes, substantially permeable membranes may comprise a monolayer or may comprise a laminated structure having two or more layers.

In an aspect, carbon dioxide permeable membranes having a permeability to carbon dioxide of between 0.6 and 2.5 cm³/cm². In an aspect, materials for construction of DEHP-free BC carbon dioxide permeable bags have a carbon dioxide permeability of greater than about 0.6 centimeters cubed per centimeters squared (cm³/cm²) at about 1 atm at 25° C. Such bags are improvements over bags prepared from conventional DEHP containing PVC which has a carbon dioxide permeability of 0.43 cm³/cm². In another aspect, DEHP-free BC carbon dioxide permeable materials having a permeability to carbon dioxide of greater than about 0.7 cm³/cm² is used for the preparation of a carbon dioxide permeable bags. In another aspect, DEHP-free BC carbon dioxide permeable materials having a permeability to carbon dioxide greater than about 0.8 cm³/cm² is used for the preparation of a carbon dioxide permeable bags. In yet another aspect, a carbon dioxide permeable material having a permeability to carbon dioxide greater than about 1.5 cm³/cm² is used for the preparation of a carbon dioxide permeable bags. In certain aspects, a carbon dioxide permeable material having a permeability to carbon dioxide greater than about 2 cm³/cm² is used for the preparation of carbon dioxide permeable bags. In other aspects, DEHP-free BC carbon dioxide permeable material having a permeability to carbon dioxide greater than about 2.2 cm³/cm² is used for the preparation of carbon dioxide permeable bags. In other aspects, carbon dioxide permeable material having a permeability to carbon dioxide of between 0.6 and 0.8, between 0.7 and 0.9, between 2 and 2.5, and between 0.6 and 2.5 cm³/cm² is used for the preparation of carbon dioxide permeable bags. In yet another aspect, carbon dioxide permeable materials are selected from materials provided by Table 1. In certain aspects, carbon dioxide permeable material is a PVC membrane having a permeability to carbon dioxide of between 0.6 and 0.8, between 0.7 and 0.9, between 2 and 2.5, and between 0.6 and 2.5 cm³/cm² is used for the preparation of carbon dioxide permeable bags. In another aspect, carbon dioxide permeable material is a polyolefin membrane having a permeability to carbon dioxide of between 0.6 and 0.8, between 0.7 and 0.9, between 2 and 2.5, and between 0.6 and 2.5 cm³/cm² is used for the preparation of carbon dioxide permeable bags. Preferably, the carbon dioxide permeable materials are DEHP-free BC carbon dioxide permeable membranes.

TABLE 1
BC Membranes with various carbon dioxide permeability

| | Plasticizer | Gas permeability of inner bag | |
|---|---|---|---|
| Membrane | (% wt/wt) | CO₂ (cm³/cm²) | O₂ (cm³/cm²) |
| PVC | DEHP (30-40%) | 0.43 | 0.09 |
| PVC | DINCH (40%) | 0.75 | 0.18 |
| PVC | BTHC | 2.02 | 0.23 |
| Polyolefin | N/A | 2.3 | 0.28 |
| (EXP 500) | | | |

In an aspect, carbon dioxide permeable membranes are also permeable to oxygen. However, in preferred aspects, carbon dioxide permeable membranes for use in the preparation of carbon dioxide permeable bags are impermeable to oxygen and are particularly suited for the preparation of outer barrier free blood storage containers. In another aspect, carbon dioxide permeable membranes having a permeability to carbon dioxide of greater than about 0.6 cm³/cm² and a permeability to oxygen of greater than 0.15 cm³/cm² at about 1 atm at 25° C. is used for the preparation of blood compatible (BC) carbon dioxide permeable bags. In another aspect, membranes having a permeability to carbon dioxide of greater than about 0.6 cm³/cm² and a permeability to oxygen of greater than 0.2 cm³/cm² at about 1 atm at 25° C. is used for the preparation of BC carbon dioxide permeable bags. In another aspect, carbon dioxide permeable membranes having a permeability to carbon dioxide of greater than about 0.6 cm³/cm² and a permeability to oxygen of less than 3.0 cm³/cm² at about 1 atm at 25° C. is used for the preparation of BC carbon dioxide permeable bags. In another aspect, carbon dioxide permeable membranes having a permeability to carbon dioxide of greater than about 0.6 cm³/cm² and a permeability to oxygen of less than 2.5 cm³/cm² at about 1 atm at 25° C. is used for the preparation of BC carbon dioxide permeable bags. In another aspect, carbon dioxide permeable membranes having a permeability to carbon dioxide of greater than about 0.6 cm³/cm² and a permeability to oxygen of less than 3 cm³/cm² at about 1 atm at 25° C. is used for the preparation of BC carbon dioxide permeable bags. In yet another aspect, carbon dioxide permeable membranes having a permeability to carbon dioxide of greater than about 0.6 cm³/cm² and a permeability to oxygen of between 0 and 3 cm³/cm² at about 1 atm at 25° C. is used for the preparation of BC carbon dioxide permeable bags.

As used herein, a DEHP-free carbon dioxide permeable bag is permeable to carbon dioxide. In certain aspects, a DEHP-free carbon dioxide permeable bag is permeable to oxygen and carbon dioxide. In other aspects, a DEHP-free carbon dioxide permeable bag is impermeable to oxygen and permeable to carbon dioxide.

In an aspect of the present disclosure other suitable DEHP-free BC carbon dioxide permeable membranes for the methods and devices according to the present disclosure include dense membranes, porous membranes, asymmetric membranes, and composite membranes. In certain aspects, suitable membranes may be multilayered membranes. In other aspects, suitable membranes are prepared from inorganic materials. Dense membranes are membranes prepared from solid materials that do not have pores or voids. Materials permeate dense membranes by processes of solution and diffusion. Examples of dense membranes include silicone membranes (polydimethyl siloxane (PDMS)). Also included and provided for in the present disclosure are porous membranes that have pores of a particular range of sizes that separate on the basis of size exclusion. Examples of porous membranes suitable for use according to the present disclosure include PVDF and polysulfone membranes.

### Outer Barrier Bag

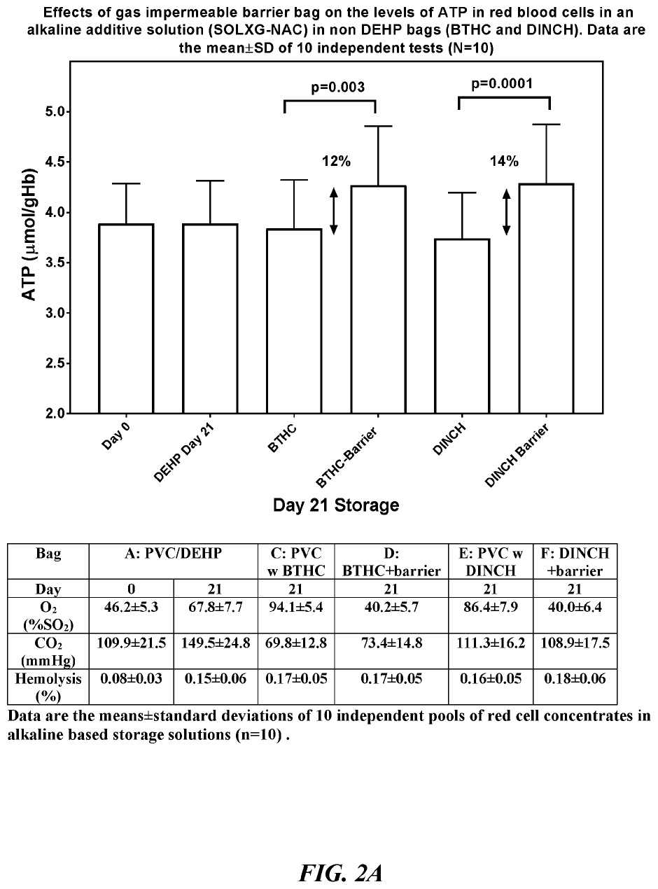

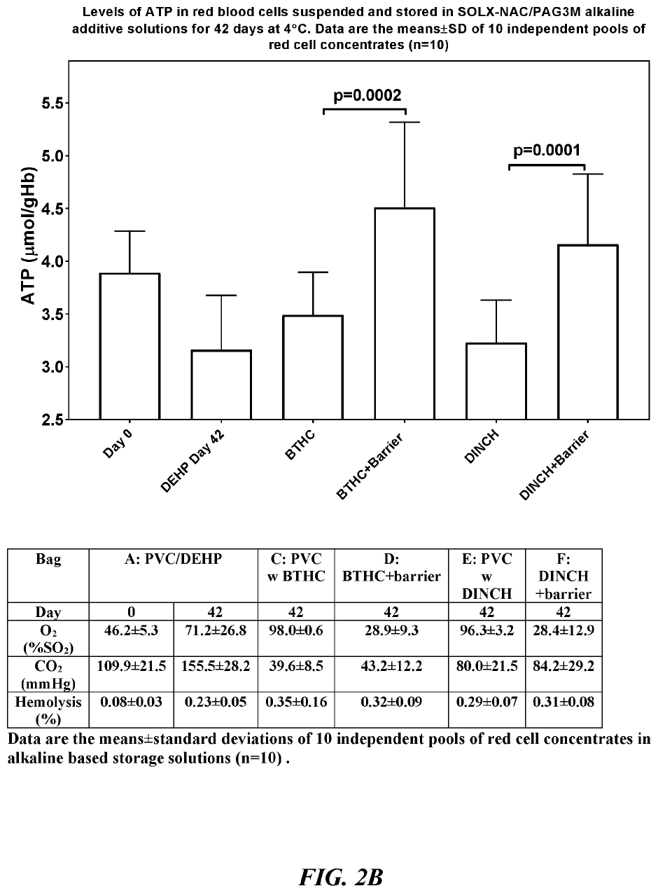

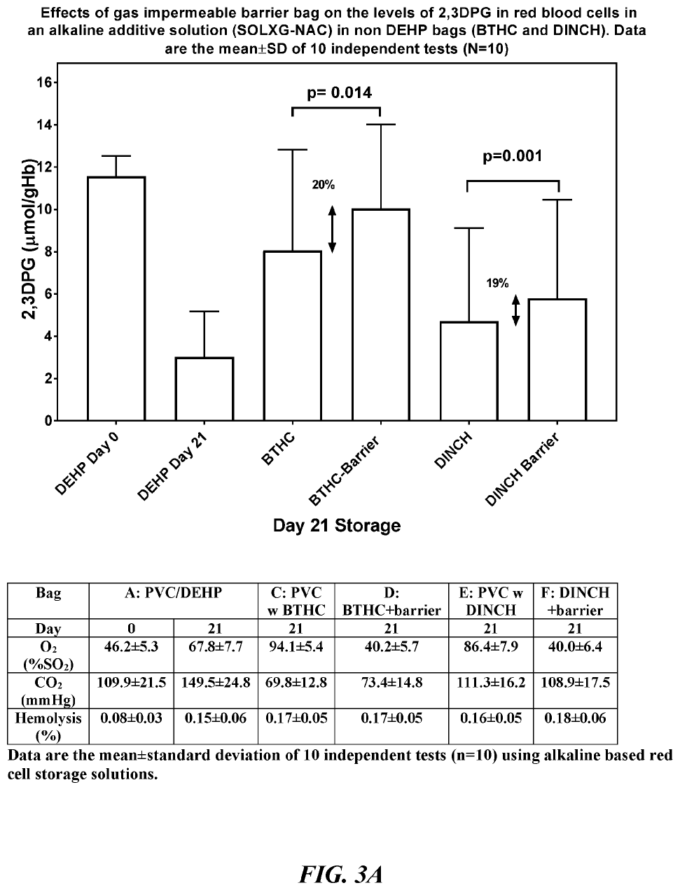

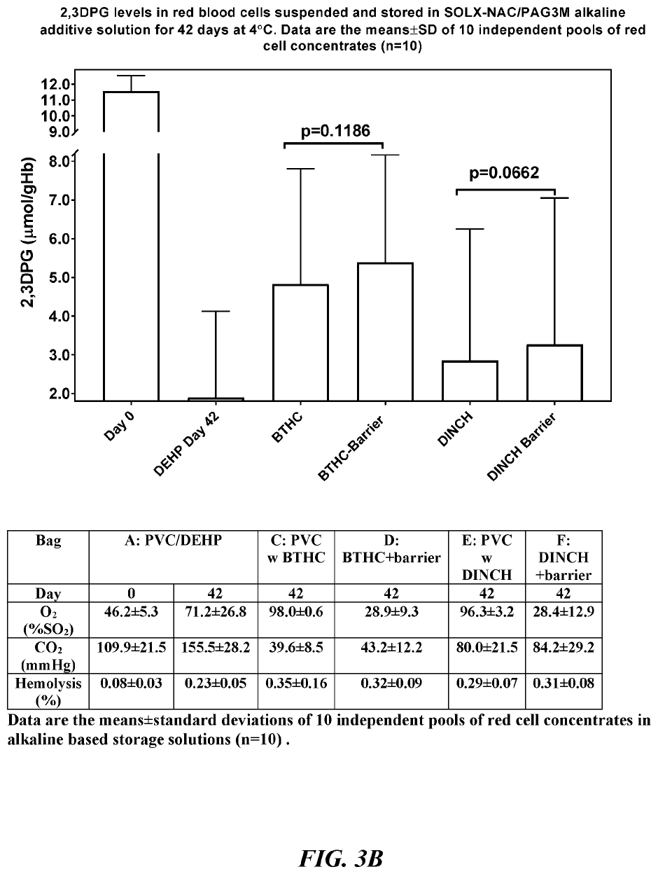

The present disclosure also provides for, and includes, a carbon dioxide permeable container for storing blood enclosed in a gas impermeable barrier bag for depleting carbon dioxide from blood during storage comprising gas impermeable barrier bag substantially impermeable to carbon dioxide, DEHP-free carbon dioxide permeable bags that is permeable to carbon dioxide, and a carbon dioxide sorbent situated within the gas impermeable barrier bag. Notably, while the addition of an outer barrier bag and sorbent can increase both 2,3-DPG levels and ATP levels, certain bags having high carbon dioxide permeability are capable of maintaining significantly higher levels of 2,3-DPG out to 42 days of storage. See FIGS. 3A, 3B Accordingly, storage bags prepared from materials having high carbon dioxide permeability and low oxygen permeability can eliminate the need for a barrier. While oxygen permeable bags benefit most from the addition of an outer barrier bag and sorbent combination, even low oxygen permeable membranes are expected to benefit as oxygen is actively removed leading to enhanced ATP levels. See FIGS. 2 to 6.

The present disclosure provides for, and includes, the preparation of gas impermeable barrier bags from a film and DEHP-free carbon dioxide permeable bags from a membrane. As used herein, membranes are generally used refer to materials used to prepare an DEHP-free carbon dioxide permeable bags and films are used to refer to materials used to prepare gas impermeable barrier bag. While it is understood that certain materials may be referred by the manufacturer as a “membrane” or may be generally known as a “membrane”, for clarity, unless otherwise indicated a film is considered substantially impermeable. A membrane comprises one or more layers of materials in the form of a sheet that allows one or more substances to pass through from one side of the sheet to the other side of the sheet. As used herein, the outer receptacles are prepared from materials that are substantially impermeable to carbon dioxide and optionally impermeable to oxygen. In certain aspects, a gas impermeable barrier bag is prepared from flexible film materials. In other aspects, a gas impermeable barrier bag is prepared from a stiff, or inflexible film material.

The present disclosure provides for, and includes, a gas impermeable barrier bag substantially impermeable to carbon dioxide. As used herein, a gas impermeable barrier bag that is substantially impermeable to carbon dioxide is sufficiently impermeable to carbon dioxide to allow no more than 10 cc of carbon dioxide inside the receptacle over a period of 3 months, and more preferably no more than 5 cc of carbon dioxide over 6 months. As used herein, the term substantially impermeable to carbon dioxide (SICO) refers to materials and compositions that provide a barrier to the passage of carbon dioxide from one side of the barrier to the other, sufficient to prevent significant increases in the partial pressure of carbon dioxide over 42 days or more.

Unless indicated otherwise, a “substantially impermeable membrane” refers to membranes that are substantially impermeable to carbon dioxide. As used herein, substantially impermeable to carbon dioxide means a permeability to carbon dioxide of less than about 1.0 cc of carbon dioxide per square meter per day. However, in certain devices and methods, the membranes may be further characterized by the permeability or impermeability to oxygen. For certain applications, the membrane material is substantially impermeable to carbon dioxide and provides a barrier to the introduction of carbon dioxide to the blood, blood component, or a blood collection kit comprised of multiple components. Such substantially impermeable membranes are generally used to prepare outer receptacles of the present disclosure. Suitable substantially impermeable membranes may also be used to prepare tubing for connective components of the devices and kits. Substantially impermeable membranes may comprise a monolayer or be laminated sheets or tubes having two or more layers.

The present disclosure also provides for, and includes, a gas impermeable barrier bag that is substantially impermeable to oxygen. As used herein, substantially impermeable to oxygen is a permeability to oxygen of less than about 1.0 cc of oxygen per square meter per day. In certain aspects, a film suitable for use in the preparation of a gas impermeable barrier bag and other elements of the present disclosure are materials characterized by a Barrer value of less than about 0.140 Barrer.

Materials and methods to prepare a gas impermeable barrier bag are known in the art. See, for example, U.S. Pat. No. 7,041,800 issued to Gawryl et al., U.S. Pat. No. 6,007,529 issued to Gustafsson et al., and U.S. Patent Application Publication No. 3013/0327677 by McDorman, each of which are hereby incorporated by reference in their entireties. Impermeable materials are routinely used in the art and any suitable material can be used. In the case of molded polymers, additives are routinely added to enhance the oxygen and carbon dioxide barrier properties. See, for example, U.S. Pat. No. 4,837,047 issued to Sato et al. For example, U.S. Pat. No. 7,431,995 issued to Smith et al. describes an oxygen- and carbon dioxide-impermeable receptacle composed of layers of ethylene vinyl alcohol copolymer and modified ethylene vinyl acetate copolymer, impermeable to oxygen and carbon dioxide ingress. In another aspect, the gas impermeable barrier bag is impermeable to oxygen and carbon dioxide.

In certain aspects, films that are substantially impermeable to carbon dioxide, oxygen, or both carbon dioxide and oxygen may be laminated films. In an aspect, a laminated film that is substantially impermeable to carbon dioxide, oxygen, or both carbon dioxide and oxygen is a laminated foil film. Film materials can be polymers or multilayer constructions that are combinations of foils and polymers. In an aspect, a laminated film may be a polyester membrane laminated with aluminum. An example of suitable aluminum laminated film, also known as a laminated foil, that is substantially impermeable to oxygen is known in the art. For example, U.S. Pat. No. 4,798,728 to Sugisawa discloses aluminum laminated foils of nylon, polyethylene, polyester, polypropylene, and vinylidene chloride. Other laminated films are known in the art. For example, U.S. Pat. No. 7,713,614 to Chow et al. discloses multilayer containers comprising an ethylene-vinyl alcohol copolymer (EVOH) resin that is substantially impermeable to oxygen. In an aspect, a gas impermeable barrier bag may be a barrier bag constructed by sealing three or four sides by means of heat sealing. The bag is constructed of a multilayer construction that includes materials that provide enhancement to carbon dioxide and oxygen barrier properties. The bag is constructed of a multilayer construction that includes materials that provide enhancement to carbon dioxide and oxygen barrier properties. Such materials include the Rollprint Clearfoil® V2 film, having an oxygen transmission rate of 0.01 cc/100 in²/24 hrs., Rollprint Clearfoil® X film, having an oxygen transmission rate of 0.004 cc/100 in²/24 hrs. and Clearfoil® Z film having an oxygen transmission rate of 0.0008 cc/100 in²/24 hrs. (Rollprint Packaging Products, Addison, Ill.). Other manufacturers make similar products with similar oxygen transmission rates, such as Renolit Solmed Wrapflex® films (American Renolit Corp., City of Commerce, Calif.). An example of suitable aluminum laminated film, also known as a laminated foil, that is substantially impermeable to oxygen is obtainable from Protective Packaging Corp. (Carrollton, Tex.).

Another approach applicable to the preparation of SICO materials includes multilayer graphitic films made by gentle chemical reduction of graphene oxide laminates with hydroiodic and ascorbic acids. See Su et al., “Impermeable barrier films and protective coatings based on reduced graphene oxide,” Nature Communications 5:4843 (2014), hereby incorporated by reference in its entirety. Nanoparticles to enhance oxygen barrier properties are also known in the art, for example, the multilayer barrier stack films provided by Tera-Barrier (Tera-Barrier Films Pte, Ltd, The Aries, Singapore) and described by Rick Lingle in Packaging Digest Magazine on Aug. 12, 3014.

In aspects according to the present disclosure, a gas impermeable barrier bag may be prepared from a gas impermeable plastic. In an embodiment, the gas impermeable plastic may be a laminate. In certain embodiments, the laminate may be a transparent barrier film, for example, a nylon polymer. In embodiment, the laminate may be a polyester film. In an embodiment, the laminate may be Mylar®. In certain embodiments, the laminate may be a metalized film. In an embodiment, the metalized film may be coated with aluminum. In another embodiment, the coating may be aluminum oxide. In another embodiment, the coating may be an ethylene vinyl alcohol copolymer (EVOH) laminated between layers of low density polyethylene (LDPE).

A gas impermeable barrier bag of the present disclosure may be formed of one or more parts prepared from a gas impermeable material including a plastic or other durable lightweight material. In some embodiments, an enclosure may be formed of more than one material. In an embodiment, a gas impermeable barrier bag may be formed of a material and coated with a gas impermeable material to prepare a gas impermeable enclosure. In an embodiment, a rigid or flexible gas impermeable barrier bag may be prepared from a plastic that may be injection molded. In embodiments according to the instant disclosure, the plastic may be selected from polystyrene, polyvinyl chloride, or nylon. In an embodiment, gas impermeable barrier bag materials may be selected from the group consisting of polyester (PES), polyethylene terephthalate (PET), polyethylene (PE), high-density polyethylene (HDPE), polyvinyl chloride (PVC), polyvinylidene chloride (PVDC), low-density polyethylene (LDPE), polypropylene (PP), polystyrene (PS), high impact polystyrene (HIPS), polyamides (PA) (e.g., nylon), acrylonitrile butadiene styrene (ABS), polycarbonate (PC), polycarbonate/acrylonitrile butadiene styrene (PC/ABS), polyurethanes (PU), melamine formaldehyde (MF), plastic material, phenolics (PF), polyetheretherketone (PEEK), polyetherimide (PEI) (Ultem), polylactic acid (PLA), polymethyl methacrylate (PMMA), polytetrafluoroethylene (PTFE), urea-formaldehyde, and ethylene vinyl alcohol copolymer (EVOH). In certain embodiments, the gas impermeable barrier bag may be polyethylene. In some embodiments, the polyethylene gas impermeable barrier bag may comprise one or more polyethylene components that are welded together. In certain aspects, the outer receptacle is comprised of a multilayer film having a polyethylene outer layer, a polyester inner layer, and an aluminum oxide barrier layer dispersed between the inner and outer layers, for example, the Clearfoil® Z film having an oxygen transmission rate of 0.0008 cc/100 in²/24 hrs. (Rollprint Packaging Products, Addison, Ill.).

The present disclosure provides for and includes the preparation of gas impermeable barrier bags using heat sealing, blow molding, and injection molding techniques. Suitable materials for preparing gas impermeable barrier bags using heat sealing, blow molding, and injection molding include PET, standard and multilayer, polypropylene, polyethylene, polycarbonate, ABS, and other polymers known to those skilled in the art. Methods to prepare blow molded and injection molded gas impermeable barrier bags are known in the art, for example, a multilayer structure comprised of a barrier layer of ethylvinyl alcohol (EVOH) or ethylvinylacetate (EVA) situated between two layers of polypropylene (PP) and offered by Kortec (Kortec, Inc., Rowley, Mass.) and also as described in U.S. Pat. No. 5,906,285 issued to Slat. Additives that strengthen the oxygen and CO₂ barrier properties of the polymers prior to molding or during their formulation or during setup are known in the art. One example is multilayer polymer co-injection resulting in a multilayer PET. Such a barrier resin is typically incorporated at the preform stage as an inner layer with PET on both sides, making PET the liquid contact layer as well as the outside layer. As provided below, suitable blow molded or injection molded gas impermeable barrier bags are impermeable to oxygen. In certain aspects, suitable heat sealed, blow molded, or injection molded gas impermeable barrier bags are substantially impermeable to both oxygen and carbon dioxide.

### Sorbents

The present disclosure provides for, and includes, sorbents capable of binding to and removing oxygen, carbon dioxide, or oxygen and carbon dioxide from an environment. Unless provided otherwise, the term “sorbent” refers to oxygen, carbon dioxide, or oxygen and carbon dioxide sorbents and scavengers. In an aspect of the present disclosure, a carbon dioxide sorbent comprises calcium oxide. Other suitable carbon dioxide sorbents include sodium hydroxide nanoparticles, calcium hydroxide and silica mixture, Calcium chloride, potassium hydroxide, perlite, activated carbons, zeolites, activated alumina, silica gel, and solid amines. In another aspect, a carbon dioxide sorbent further comprises an oxygen sorbent.

As used herein, “oxygen scavenger” or “oxygen sorbent” is a material that binds irreversibly to or combines with O₂ under the conditions of use. As used herein, “carbon dioxide scavenger” or “carbon dioxide sorbent” is a material that binds irreversibly to or combines with CO₂ under the conditions of use. The term “oxygen sorbent” or “carbon dioxide sorbent” may be used interchangeably herein with “oxygen scavenger” or carbon dioxide,” respectively. In certain aspects according the present disclosure, a material may bind to or combines with oxygen or carbon dioxide irreversibly. In other aspects, oxygen or carbon dioxide may bind to a sorbent material and have a very slow rate of release, k_{off}. In an aspect, the oxygen or carbon dioxide may chemically react with some component of the material and be converted into another compound. Any material where the off-rate of bound oxygen is much less than the residence time of the blood can serve as an oxygen scavenger. Further, any material where the off-rate of bound carbon dioxide is much less than the residence time of the blood can serve as a carbon dioxide scavenger.

As used herein, the amount of sorbent is provided as having a certain binding capacity of oxygen as measured by volume (e.g., cubic centimeters (cc) or milliliters (ml)) at standard temperature and pressure (e.g., 0° C. (273.15 Kelvin) and 1.01×10⁵ pa (100 kPa, 1 bar, 0.986 atm, 760 mmHg) of pressure). In other aspects, oxygen sorbents and scavengers are further capable of binding to and removing carbon dioxide from an environment. In certain aspects, sorbent may be a mixture of non-toxic inorganic and/or organic salts and ferrous iron or other materials with high reactivity toward oxygen, carbon dioxide, or oxygen and carbon dioxide. In certain aspects, an oxygen sorbent or scavenger is combined with a carbon dioxide sorbent. In other aspects, the presence or absence of carbon dioxide binding capabilities of an oxygen sorbent is not necessary.

Suitable oxygen sorbents or scavengers are known in the art. Suitable oxygen sorbents according to the present disclosure have minimum oxygen adsorption rates of 0.44 ml/min. Sorbents having suitable adsorption profiles bind at least 45 ml 02 within 60 minutes, 70 ml 02 within 120 minutes, and 80 ml 02 within 180 minutes. Suitable sorbents may have both higher capacity and binding rates.

Non-limiting examples of oxygen scavengers or sorbents include iron powders and organic compounds. Examples of 02 sorbents include chelates of cobalt, iron, and Schiff bases. Additional non-limiting examples for 02 sorbents may be found in U.S. Pat. No. 7,347,887 issued to Bulow et al., U.S. Pat. No. 5,208,335, issued to Ramprasad et al., and U.S. Pat. No. 4,654,053 issued to Sievers et al.; each of which is hereby incorporated by reference in their entireties. Oxygen sorbent materials may be formed into or incorporated in fibers, microfibers, microspheres, microparticles, and foams.

In certain aspects, suitable sorbents include those obtainable from Multisorb Technologies (Buffalo, N.Y.), Sorbent Systems/Impak Corporation (Los Angeles, Calif.) or Mitsubishi Gas Chemical America (MGC) (New York, N.Y.). Exemplary oxygen sorbents include Multisorb Technologies StabilOx® packets, Sorbent Systems P/N SF100PK100 100 cc oxygen absorber, and Mitsubishi Gas Chemical America Ageless® SS-200 oxygen absorber. MGC also provides sorbents suitable for the methods and devices of the present disclosure. Such suitable oxygen sorbents include the MGC Ageless® and SS-200 oxygen absorber.

In aspects according to the present disclosure, a sorbent may be an oxidizable organic polymer having a polymeric backbone and a plurality of pendant groups. Examples of sorbents with a polymeric backbone include a saturated hydrocarbon (<0.01% carbon-carbon double bonds). In some aspects, the backbone can contain monomers of ethylene or styrene. In an aspect, a polymeric backbone may be ethylenic. In another aspect, an oxidizable organic compound may be ethylene/vinyl cyclohexene copolymer (EVCH). Additional examples of substituted moieties and catalysts are provided in U.S. Patent Publication No. 2003/0183801 by Yang et al., hereby incorporated by reference in its entirety. In additional aspects, an oxidizable organic polymer can also comprise substituted hydrocarbon moieties. Examples of oxygen scavenging polymers include those described by Ching et al., International Patent Publication WO99/48963, hereby incorporated by reference in its entirety. Oxygen scavenging materials may include those provided in U.S. Pat. No. 7,754,798 issued to Ebner et al., U.S. Pat. No. 7,452,601 issued to Ebner et al., or U.S. Pat. No. 6,387,461 issued to Ebner et al., each of which are hereby incorporated by reference in their entireties.

As used herein, sorbents of the present disclosure may be either free or contained in a permeable enclosure, container, envelope, etc. In certain aspects, sorbent is provided in one or more sachets made of materials having high porosity and essentially no resistance to the transport of gases. Examples of such materials include spun polyester films, perforated metallic foils, and combinations thereof.

The present disclosure further includes, and provides for, sorbent incorporated as one or more laminated layers of an outer article substantially impermeable to oxygen. Polymeric sorbents such as those described above may be laminated to sheets used to prepare an outer receptacle using methods known in the art, including soft contact lamination, thermal lamination, or solvent lamination.

The present disclosure further includes, and provides for, sorbents formed inside the pores of porous micro-glass fibers or encapsulated in other inert materials. The encapsulation of transition-metal complexes within the pores of a porous material may be achieved by using a ship-in-a-bottle synthesis in which the final molecule is prepared inside the pores by reacting smaller precursors. Examples of such encapsulated sorbents are known in the art, for example, as described by Kuraoka, et al., “Ship-in-a-bottle synthesis of a cobalt phthalocyanine/porous glass composite membrane for oxygen separation,” Journal of Membrane Science, 286(1-2):12-14 (2006), herein incorporated by reference in its entirety. In some aspects, porous glass fibers may be manufactured as provided in U.S. Pat. No. 4,748,121 issued to Beaver et al., herein incorporated by reference in its entirety. In another aspect, a sorbent can be formed as a porous sheet product using papermaking/non-woven wet-laid equipment. Sheets with O₂ scavenging formulations may be as described in U.S. Pat. No. 4,769,175 issued to Inoue, herein incorporated by reference in its entirety, which can be formed and then encapsulated with a silicone film.

As used herein, “carbon dioxide scavenger” or “carbon dioxide sorbent” is a material that binds to or combines with carbon dioxide under the conditions of use. The term “carbon dioxide sorbent” may be used interchangeably herein with “carbon dioxide scavenger.” In certain aspects, carbon dioxide sorbents may be non-reactive, or minimally reactive with oxygen. In other embodiments, oxygen sorbents may exhibit a secondary functionality of carbon dioxide scavenging. Carbon dioxide scavengers include metal oxides and metal hydroxides. Metal oxides react with water to produce metal hydroxides. The metal hydroxide reacts with carbon dioxide to form water and a metal carbonate. In certain aspects according the present disclosure, a material may bind to or combine with CO₂ irreversibly. In aspects according to the present disclosure, a material may bind CO₂ with higher affinity than hemoglobin. In other aspects, a sorbent material may bind CO₂ with high affinity such that the carbonic acid present in the blood or RBC cytoplasm is released and absorbed by the sorbent. In other aspects, CO₂ binds to a sorbent material and has a very slow rate of release, k_{off}. In an aspect, the carbon dioxide can chemically react with some component of the material and be converted into another compound.

Carbon dioxide scavengers are known in the art. In certain aspects according to the present disclosure, a carbon dioxide scavenger may be calcium oxide. Reaction of calcium oxide with water produces calcium hydroxide that may react with carbon dioxide to form calcium carbonate and water. In certain aspects according to the present disclosure, the water for the production of calcium hydroxide is obtained via diffusion of blood derived water vapor through the inner oxygen permeable container. In another aspect, the water may be provided by the environment through the outer receptacle that is substantially impermeable to oxygen. In yet another aspect, the water may be included with the outer receptacle of the carbon dioxide permeable container for storing blood enclosed in a gas impermeable barrier bag.

Non-limiting examples of CO₂ scavengers include oxygen scavengers and carbon dioxide scavengers provided by Multisorb Technologies (Buffalo, N.Y.). Oxygen scavengers may exhibit a secondary functionality of carbon dioxide scavenging.

In aspects according to the present disclosure, O₂ depletion media and CO₂ depletion media may be blended to a desired ratio to achieve desired results.

The present disclosure further includes and provides for scavengers or sorbents contained in sachets. As used herein, a “sachet” is any enclosure that encloses and contains an oxygen sorbent, a carbon dioxide sorbent, or a combination of oxygen and carbon dioxide sorbent(s). Sachets according the present disclosure are contained within overwrap material that is both oxygen and carbon dioxide permeable. In certain embodiments, the overwrap material may be a combination of two or more materials, at least one of the materials being oxygen and carbon dioxide permeable. Suitable overwrap materials have a known biocompatible profile or meet International Organization of Standardization (ISO) 10993.

Sachets are sealed so that the sorbent contents are wholly contained within the overwrap material and do not allow the sorbent to leak or otherwise exit its overwrap package. Sachets may take any shape, though typically take a rectangular or square shape. In an aspect, the sachet is about 50×60 mm. In an aspect, the oxygen sorbent binds 30 cc oxygen per sachet at standard temperature and pressure (STP). In an aspect, the oxygen sorbent binds 60 cc oxygen per sachet at STP. In an aspect, the oxygen sorbent binds 120 cc oxygen per sachet at STP. In an aspect, the oxygen sorbent binds from 30 to 120 cc oxygen per sachet at STP. In an aspect, the oxygen sorbent binds from 30 to 120 cc oxygen per sachet at STP. In an aspect, the oxygen sorbent binds from 50 to 200 cc oxygen per sachet at STP. In certain aspects according to the present disclosure, a sachet has a total oxygen adsorption capacity of 100 cc 02 at STP. In certain other aspects of the present disclosure, a sachet has a total oxygen absorption capacity of at least 200 cc 02 at STP.

In aspects according to the present disclosure, the oxygen sorbent may be provided in one or more sachets. In another aspect, an oxygen sorbent is provided in a single larger sachet. In other aspects, the oxygen sorbent is provided in two sachets distributed within the headspace between the DEHP-free carbon dioxide permeable bags and the gas impermeable barrier bag. In yet other aspects, the oxygen sorbent is provided in four sachets distributed within the headspace between the DEHP-free carbon dioxide permeable bags and the gas impermeable barrier bag. In aspects according to the present disclosure, a carbon dioxide permeable container for storing blood enclosed in a gas impermeable barrier bag may comprise 2 to 20 sorbent packages.

In some aspects according to the present disclosure, a carbon dioxide permeable container for storing blood is enclosed in a gas impermeable barrier bag includes from 1 to 50 grams of sorbent contained in one or more sachets. In an aspect, a carbon dioxide permeable container for storing blood enclosed in a gas impermeable barrier bag includes from 1 to 100 grams of sorbent contained in one or more sachets. In an aspect, a carbon dioxide permeable container for storing blood enclosed in a gas impermeable barrier bag includes from 25 to 75 grams of sorbent contained in one or more sachets. In a further aspect, a carbon dioxide permeable container for storing blood enclosed in a gas impermeable barrier bag includes about 25 grams of sorbent. In yet another aspect, a carbon dioxide permeable container for storing blood enclosed in a gas impermeable barrier bag includes about 50 grams of sorbent. In an aspect, a carbon dioxide permeable container for storing blood enclosed in a gas impermeable barrier bag includes about 35 or 45 grams of sorbent contained in one or more sachets. In an aspect, a carbon dioxide permeable container for storing blood enclosed in a gas impermeable barrier bag includes about 10 or 15 grams of sorbent contained in one or more sachets. The sachets can be square, rectangular, circular, or elliptical and have a perimeter of 40 to 150 mm.

Sachets according to the present disclosure may further include a carbon dioxide sorbent. In an aspect, an oxygen sorbent also provides for carbon dioxide adsorption. In an aspect, the oxygen sorbent binds 30 cc carbon dioxide at STP. In an aspect, the oxygen sorbent binds at least 170 cc oxygen and at least 30 cc carbon dioxide, where both gases are measured at STP.

### Additive Solutions/Compositions

The present disclosure provides for and includes compositions including additive solutions and methods for adding an additive solution to a blood product. In another aspect, compositions and methods include adding an additive solution to red blood cells. In another aspect, compositions and methods include adding additive solutions to platelets. In another aspect, compositions and methods include adding additive solution to whole blood. In another aspect, compositions and methods include adding an additive solution to packed RBCs to form a suspension.

In certain aspects, the additive solution may be selected from the group consisting of additive solution (AS)-1, AS-3 (Nutricel), AS-5, AS7 (SOLX), SAGM, PAGG-SM, PAGG-GM, MAP, ESOL, EAS61, OFAS1, and OFAS3, alone or in combination. See Table 2.

TABLE 2
Additive solutions

| Additive | |
|---|---|
| Solution | Reference |
| AS-1 | Heaton et al., “Use of Adsol preservation |
| (Adsol) | solution for prolonged storage of low viscosity |
| | AS-1 red blood cells,” Br J Haematol., |
| | 57(3): 467-78 (1984). |
| AS-3 | Simon et al., “Effects of AS-3 nutrient- |
| (Nutricel ®) | additive solution on 42 and 49 days of storage |
| | of red cells,” Transfusion. 27(2): 178-82 (1987). |
| AS-5 | Cicha et al., “Gamma-ray-irradiated red blood |
| (Optisol) | cells stored in mannitol-adenine-phosphate medium: |
| | rheological evaluation and susceptibility to |
| | oxidative stress,” Vox Sang. 79(2): 75-82 (2000). |
| AS7 | Cancelas et al., “Additive solution-7 reduces |
| (SOLX) | the red blood cell cold storage lesion,” |
| | Transfusion. 55(3): 491-8 (2015). |
| EAS61 | Hess et al., “Successful storage of RBCs for |
| | 9 weeks in a new additive solution,” |
| | Transfusion 40(8): 1007-1011 (2000). |
| Erythrosol-5 | Radwanski et al., “Red cell storage in E-Sol |
| (ESOL-5) | 5 and Adsol additive solutions: paired comparison |
| | using mixed and non-mixed study designs,” |
| | Vox Sang. 106(4): 322-9 (2014). |
| Erythrosol-5G | Described herein |
| Erythrosol-5GG | Described herein |
| MAP | Sasakawa et al., “Development of additive |
| | solution MAP for storage of red cell concentrates,” |
| | Japanese Journal of Transfusion Medicine |
| | 37(3): 398-403 (1991). |
| OFAS3 | Dumont et al, “Anaerobic storage of red blood |
| | cells in a novel additive solution improves in |
| | vivo recovery,” Transfusion 49(3): 458-464 (2009). |
| PAGG-GM | Burger et al., “An improved red blood cell |
| | additive solution maintains 2,3-diphosphoglycerate |
| | and adenosine triphosphate levels by an enhancing |
| | effect on phosphofructokinase activity during cold |
| | storage,” Transfusion 50(11): 2386-2392 (2010). |
| PAGG-SM | Walker et al., “49 day storage of erythrocyte |
| | concentrates in blood bags with the PAGGS-mannitol |
| | solution,” Beitr Zur Infusionstherapie |
| | Contrib Infus Ther. 26: 55-9 (1990). |
| SAGM | Högman et al., “Red cell preservation in |
| | protein-poor media. III. Protection against in |
| | vitro hemolysis,” Vox Sang. 41(5-6): 274-81 (1981). |

In a further aspect, the additive solution may have a pH of from 5.0 to 7.0. In another aspect, the additive solution has a pH from 7.0 to 9.0. In another aspect, the additive may include an antioxidant. In some aspects according the present disclosure, the antioxidant may be quercetin, alpha-tocopherol, ascorbic acid, or enzyme inhibitors for oxidases. In another aspect, an additive solution further comprises quercetin. In another aspect, an additive solution further comprises alpha-tocopherol. In another aspect, an additive solution further comprises ascorbic acid. In yet another aspect an additive solution further comprises enzyme inhibitors for oxidases. In another aspect, an additive solution comprises N-acetylcysteine; 6-Hydroxy-2,5,7,8-tetramethylchroman-2-carboxylic acid (Trolox); and 1-ascorbic acid (vitamin C).

In an aspect of the present disclosure, an additive solution is selected from the group consisting of AS7, AS7G-NAC, or AS7-NAC with gluconate (AS7GG-NAC) as provided in Table 3. In an aspect of the present disclosure, an additive solution comprises sodium bicarbonate (NaHCO₃); sodium phosphate dibasic (Na₂HPO₄); adenine; guanosine; glucose; mannitol; N-acetyl-cysteine; 6-Hydroxy-2,5,7,8-tetramethylchroman-2-carboxylic acid (Trolox); and 1-ascorbic acid (vitamin C). In another aspect, an additive solution comprises between 10 and 60 mM sodium bicarbonate (NaHCO₃); between 10 and 20 mM sodium phosphate dibasic (Na₂HPO₄); between 0 and 5 mM adenine; between 0 and 5 mM guanosine; between 50 and 100 mM glucose; between 40 and 80 mM mannitol; between 0 and 1 mM N-acetyl-cysteine; between 0 and 1 mM 6-Hydroxy-2,5,7,8-tetramethylchroman-2-carboxylic acid; and between 0 and 1 mM 1-ascorbic acid. In certain aspects, an additive solution comprises 40 mM sodium bicarbonate (NaHCO₃); 12 mM sodium phosphate dibasic (Na₂HPO₄); 2 mM adenine; 1.4 mM guanosine; 80 mM glucose; 55 mM mannitol; 0.5 mM N-acetyl-cysteine; 0.5 mM 6-Hydroxy-2,5,7,8-tetramethylchroman-2-carboxylic acid; and 0.25 mM 1-ascorbic acid. In other aspects, an additive solution comprises 40 mM sodium bicarbonate (NaHCO₃); between 12 mM sodium phosphate dibasic (Na₂HPO₄); 2 mM adenine; 1.4 mM guanosine; 80 mM glucose; 55 mM mannitol; 0.5 mM N-acetyl-cysteine; 0.5 mM 6-Hydroxy-2,5,7,8-tetramethylchroman-2-carboxylic acid; 0.25 mM 1-ascorbic acid; and 4 mM gluconate.

TABLE 3
Formulations of AS7, AS7G-NAC and AS7GG-NAC

| Chemicals | AS7 | AS7G-NAC | | AS7GG-NAC | |
|---|---|---|---|---|---|
| NaHCO₃ | 26 mM | 40 | mM | 40 | mM |
| Na₂HPO₄ | 12 mM | 12 | mM | 12 | mM |
| Gluconate | — | — | | 4 | mM |
| Adenine | 2 mM | 2 | mM | 2 | mM |
| Guanosine | — | 1.4 | mM | 1.4 | mM |
| Glucose | 80 mM | 80 | mM | 80 | mM |
| Mannitol | 55 mM | 55 | mM | 55 | mM |
| N-Acetyl-Cysteine | — | 0.50 | mM | 0.50 | mM |
| Trolox | — | 0.50 | mM | 0.50 | mM |
| Vitamin C | — | 0.25 | mM | 0.25 | mM |

| | | | |
|---|---|---|---|
| pH (Adjusted 10M NaOH) | 8.5 | 8.75 | 8.75 |

In another aspect of the present disclosure, an additive solution is selected from the group consisting of Erythrosol-5, Erythrosol-5G with 5 mM gluconate (Erythrosol-5GG), or Erythrosol-5G without gluconate, as provided in Table 4. In another aspect, an additive solution further comprises N-acetyl-cysteine; 6-Hydroxy-2,5,7,8-tetramethylchroman-2-carboxylic acid (Trolox); and 1-ascorbic acid (vitamin C). In an aspect of the present disclosure, an additive solution comprises between 10 and 40 mM Na₂HPO₄, between 10 and 40 mM sodium citrate, between 0.5 and 3 mM adenine, between 30 and 60 mM glucose, and between 80 and 130 mM mannitol. In another aspect, an additive solution comprises between 10 and 40 mM Na₂HPO4, between 10 and 40 mM sodium citrate, between 0.5 and 3 mM adenine, between 30 and 60 mM glucose, between 80 and 130 mM mannitol, and between 0.5 and 3 mM guanosine. In another aspect, an additive solution also comprises between 2 and 8 mM gluconate. In yet another aspect, an additive solution has a pH between 7.5 and 9. In another aspect, an additive solution has a pH of at least 7.0, 7.2, 7.4, 7.5, 7.6, 7.8, 8.0, 8.2, 8.4, 8.5, 8.6, and 8.8. In another aspect, an additive solution has a pH of from 7.0 to 7.5, from 7.5 to 8, from 8 to 8.2, from 8 to 8.4, from 8 to 8.6, from 8 to 8.8, from 8.4 to 9.

In certain aspects of the present disclosure, an additive solution comprises 20 mM Na₂HPO₄, 25 mM sodium citrate, 1.5 mM adenine, 45.5 mM glucose, 110 mM mannitol and a pH of 8.8. In another aspect, an additive solution comprises 20 mM Na₂HPO₄, 25 mM sodium citrate, 1.5 mM adenine, 45.5 mM glucose, 110 mM mannitol, 5 mM gluconate and a pH of 8.8.

TABLE 4
Formulations of an alkaline additive solution

| | Erythrosol-5 | Erythrosol-5GG | Erythrosol-5G |
|---|---|---|---|
| NaCl (mM) | | | |
| Na₂HPO₄(mM) | 20 | 20 | 20 |
| NaH₂PO₄(mM) | | | |
| Na-citrate (mM) | 25 | 25 | 25 |
| Adenine (mM) | 1.5 | 1.5 | 1.5 |
| Guanosine (mM) | | 1.5 | 1.5 |
| Gluconate (mM) | | 5 | |
| Glucose | 45.5 | 45.5 | 45.5 |
| Mannitol (mM) | 110 | 110 | 110 |
| pH | 8.8 | 8.8 | 8.8 |

The present disclosure provides for, and includes, a composition comprising: a blood product selected from the group consisting of whole blood, platelets, and leukocytes; and an additive solution comprising sodium bicarbonate (NaHCO₃); sodium phosphate dibasic (Na₂HPO₄); adenine; guanosine; glucose; mannitol; N-acetyl-cysteine; 6-Hydroxy-2,5,7,8-tetramethylchroman-2-carboxylic acid (Trolox); and 1-ascorbic acid (vitamin C).

The present disclosure further provides for, and includes, a composition comprising a blood product selected from the group consisting of whole blood, platelets, and leukocytes having a pCO₂ of less than 125 mmHg; and an additive solution comprising a concentration of sodium phosphate dibasic (Na₂HPO₄), sodium citrate, adenine, glucose, and mannitol.

The present disclosure further provides for, and includes, a stored blood product comprising a pCO₂ of less than 125 mmHg, a % SO₂ of greater than 20%, and an additive solution comprising 40 mM sodium bicarbonate (NaHCO₃); 12 mM sodium phosphate dibasic (Na₂HPO₄); 2 mM adenine; 1.4 mM guanosine; 80 mM glucose; 55 mM mannitol; 0.5 mM N-acetyl-cysteine; 0.5 mM 6-Hydroxy-2,5,7,8-tetramethylchroman-2-carboxylic acid (Trolox); and 0.25 mM 1-ascorbic acid (vitamin C). In an aspect, the stored blood composition further comprises an ATP concentration of at least 4 μmol/g Hb after 42 days of storage, a CO2 concentration of less than 60 mmHg. In an aspect, the stored blood composition further comprises an 2,3-DPG concentration of at least 6 mol/g Hb after 21 days of storage. In an aspect, the stored blood composition further comprises an 2,3-DPG concentration of at least 4 μmol/gHb after 42 days of storage.

In another aspect, the blood product has a pCO₂ of less than 100 mmHg, a % SO₂ of greater than 20%, an additive solution comprising 40 mM sodium bicarbonate (NaHCO₃); 12 mM sodium phosphate dibasic (Na₂HPO₄); 2 mM adenine; 1.4 mM guanosine; 80 mM glucose; 55 mM mannitol; 0.5 mM N-acetyl-cysteine; 0.5 mM 6-Hydroxy-2,5,7,8-tetramethylchroman-2-carboxylic acid (Trolox); and 0.25 mM 1-ascorbic acid (vitamin C). In an aspect, the stored blood composition further comprises an ATP concentration of at least 4 μmol/gHb after 42 days of storage and pCO₂ of less than 50 mmHg and % SO₂ of less than 50% compared to 3 μmol/gHb and pCO₂ of 92 mmHg and % SO₂ of 89% in conventionally stored red cells on day 42 of storage. In an aspect, the stored blood composition further comprises an 2,3-DPG concentration of at least 6 mol/gHb after 21 days of storage. In an aspect, the stored blood composition further comprises an 2,3-DPG concentration of at least 4 mol/gHb after 42 days of storage at pCO₂ of less than 50 mmHg compared to conventionally stored red blood cell with a 2,3DPG concentration of 0.5 μmol/gHb and pCO₂ of 92 mmHg on day 42 of storage.

In another aspect, the blood product has a pCO₂ of less than 75 mmHg, a % SO₂ of greater than 20%, an additive solution comprising 40 mM sodium bicarbonate (NaHCO₃); 12 mM sodium phosphate dibasic (Na₂HPO₄); 2 mM adenine; 1.4 mM guanosine; 80 mM glucose; 55 mM mannitol; 0.5 mM N-acetyl-cysteine; 0.5 mM 6-Hydroxy-2,5,7,8-tetramethylchroman-2-carboxylic acid (Trolox); and 0.25 mM 1-ascorbic acid (vitamin C).

In another aspect, the blood product has a pCO₂ of less than 25 mmHg, a % SO₂ of greater than 20%, and an additive solution comprising 40 mM sodium bicarbonate (NaHCO₃); 12 mM sodium phosphate dibasic (Na₂HPO₄); 2 mM adenine; 1.4 mM guanosine; 80 mM glucose; 55 mM mannitol; 0.5 mM N-acetyl-cysteine; 0.5 mM 6-Hydroxy-2,5,7,8-tetramethylchroman-2-carboxylic acid (Trolox); and 0.25 mM 1-ascorbic acid (vitamin C).

In another aspect, the blood product has a pCO₂ of less than 125 mmHg, % SO₂ of between 5 and 30%, an additive solution comprising 40 mM sodium bicarbonate (NaHCO₃); 12 mM sodium phosphate dibasic (Na₂HPO₄); 2 mM adenine; 1.4 mM guanosine; 80 mM glucose; 55 mM mannitol; 0.5 mM N-acetyl-cysteine; 0.5 mM 6-Hydroxy-2,5,7,8-tetramethylchroman-2-carboxylic acid (Trolox); and 0.25 mM 1-ascorbic acid (vitamin C).

In another aspect, the blood product has a pCO₂ of less than 100 mmHg, a % SO₂ of between 5 and 30%, and an additive solution comprising 40 mM sodium bicarbonate (NaHCO₃); 12 mM sodium phosphate dibasic (Na₂HPO₄); 2 mM adenine; 1.4 mM guanosine; 80 mM glucose; 55 mM mannitol; 0.5 mM N-acetyl-cysteine; 0.5 mM 6-Hydroxy-2,5,7,8-tetramethylchroman-2-carboxylic acid (Trolox); and 0.25 mM 1-ascorbic acid (vitamin C).

In another aspect, the blood product has a pCO₂ of less than 75 mmHg, a % SO₂ of between 5 and 30%, an additive solution comprising 40 mM sodium bicarbonate (NaHCO₃); 12 mM sodium phosphate dibasic (Na₂HPO₄); 2 mM adenine; 1.4 mM guanosine; 80 mM glucose; 55 mM mannitol; 0.5 mM N-acetyl-cysteine; 0.5 mM 6-Hydroxy-2,5,7,8-tetramethylchroman-2-carboxylic acid (Trolox); and 0.25 mM 1-ascorbic acid (vitamin C).

In another aspect, the blood product has a pCO₂ of less than 50 mmHg, a % SO₂ of between 5 and 30%, an additive solution comprising 40 mM sodium bicarbonate (NaHCO₃); 12 mM sodium phosphate dibasic (Na₂HPO₄); 2 mM adenine; 1.4 mM guanosine; 80 mM glucose; 55 mM mannitol; 0.5 mM N-acetyl-cysteine; 0.5 mM 6-Hydroxy-2,5,7,8-tetramethylchroman carboxylic acid (Trolox); and 0.25 mM 1-ascorbic acid (vitamin C).

In another aspect, the blood product has a pCO₂ of less than 25 mmHg, a % SO₂ of between 5 and 30%, and an additive solution comprising 40 mM sodium bicarbonate (NaHCO₃); 12 mM sodium phosphate dibasic (Na₂HPO₄); 2 mM adenine; 1.4 mM guanosine; 80 mM glucose; 55 mM mannitol; 0.5 mM N-acetyl-cysteine; 0.5 mM 6-Hydroxy-2,5,7,8-tetramethylchroman-2-carboxylic acid (Trolox); and 0.25 mM 1-ascorbic acid (vitamin C).

In yet another aspect, the blood product has a pCO₂ of less than 50 mmHg, a % SO₂ of between 5 and 30%, an additive solution provided in Table 3 or Table 4.

The present disclosure provides for, and includes, the following embodiments:

Embodiment 1. A method for the storage of a blood product comprising: obtaining a blood product having a % SO₂ of greater than 30%; adding an additive solution to said blood product to prepare a storable blood product; and storing said storable blood product in a di-2-ethylhexyl phthalate free (DEHP-free) blood compatible (BC) carbon dioxide permeable bag comprising a gas permeability for carbon dioxide of at least 0.62 centimeters cubed per centimeters squared (cm³/cm²) at about 1 atm at 25° C.

Embodiment 2. The method of embodiment 1, wherein said storable blood product is not deoxygenated prior to said storing.

Embodiment 3. The method of any one of any one of embodiments 1 or 2, wherein said storable blood product is not deoxygenated during said storing.

Embodiment 4. The method of embodiment 2, comprising depleting oxygen from said storable blood product during storage.

Embodiment 5. The method of any one of embodiments 1 to 4, wherein said BC carbon dioxide permeable bag comprises an oxygen permeability of less than 0.3 cm cm³/cm².

Embodiment 6. The method of any one of embodiments 1 to 5, wherein said BC carbon dioxide permeable bag does not comprise di(2-ethylhexyl) terephthalate (DEHT).

Embodiment 7. The method of any one of embodiments 1 to 6, wherein said BC carbon dioxide permeable bag comprises 1,2-Cyclohexane dicarboxylic acid diisononyl ester (DINCH) or butyryltrihexylcitrate (BTHC) as a plasticizer.

Embodiment 8. The method of any one of embodiments 1 to 7, wherein said BC carbon dioxide permeable bag is enclosed in an outer bag impermeable to oxygen and carbon dioxide.

Embodiment 9. The method of any one of embodiments 1 to 8, wherein said outer bag further encloses a carbon dioxide sorbent placed between said BC carbon dioxide permeable bag and said outer bag.

Embodiment 10. The method of any one of embodiments 1 to 9, wherein 2,3-DPG levels are increased by at least 10% in said storable blood product during said storing compared to 2,3-DPG levels of a conventionally stored blood product.

Embodiment 11. The method of any one of embodiments 1 to 10, wherein 2,3-DPG levels are increased by at least 15% in said storable blood product during said storing compared to 2,3-DPG levels of a conventionally stored blood product.

Embodiment 12. The method of any one of embodiments 1 to 11, wherein ATP levels are increased in said storable blood product during said storing compared to ATP levels of a conventionally stored blood product.

Embodiment 13. The method of embodiment 12, wherein ATP levels are increased by at least 10% in said storable blood product during said storing compared to ATP levels of a conventionally stored blood product.

Embodiment 14. The method of any one of embodiments 1 to 13, wherein said additive solution has a pH of between 7.0 and 8.5.

Embodiment 15. The method of any one of embodiments 1 to 14, wherein said additive solution has a pH of at least 8.5.

Embodiment 16. The method of embodiment 9, wherein said carbon dioxide sorbent further comprises an oxygen sorbent.

Embodiment 17. The method of any one of embodiments 1 to 16, wherein said BC carbon dioxide permeable bag comprises a gas permeability for oxygen of at least 0.05 centimeter³ (cm³)/cm²/atm 24 hours (hrs.) at 25° C.

Embodiment 18. The method of embodiment 17, wherein said BC carbon dioxide permeable bag comprises a gas permeability for oxygen of at least 0.15 cm³/cm²/atm 24 hrs. at 25° C.

Embodiment 19. The method of embodiment 18, wherein said BC carbon dioxide permeable bag comprises a gas permeability for oxygen of about 0.22 cm³/cm²/atm 24 hrs. at 25° C.

Embodiment 20. The method of any one of embodiments 1 to 19, wherein said blood product comprises greater than 15% saturated oxygen (SO2) during said storing of up to 42 days.

Embodiment 21. The method of embodiment 20, wherein said blood product comprises greater than 20% SO2 during said storing of up to 42 days.

Embodiment 22. The method of any one of embodiments 1 to 21, wherein said blood product comprises less than 125 mmHg pCO₂ during said storing of up to 42 days.

Embodiment 23. The method of embodiment 22, wherein said blood product comprises less than 100 mmHg pCO₂ during said storing of up to 42 days.

Embodiment 24. The method of embodiment 23, wherein said blood product comprises less than 75 mmHg pCO₂ during said storing for up to 42 days.

Embodiment 25. The method of embodiment 24, wherein said blood product comprises less than 50 mmHg pCO₂ during said storing of up to 42 days.

Embodiment 26. The method of any one of embodiments 1 to 25, wherein said storing is for less than 42 days.

Embodiment 27. The method of embodiment any one of embodiments 10, 11, 12, or 13, wherein said storing is for less than 28 days.

Embodiment 28. The method of embodiment any one of embodiments 10, 11, 12, or 13, wherein said storing is for less than 21 days.

Embodiment 29. The method of embodiment any one of embodiments 10, 11, 12, or 13, wherein said storing is for less than 14 days.

Embodiment 30. The method of embodiment any one of embodiments 10, 11, 12, or 13, wherein said storing is for less than 7 days.

Embodiment 31. The method of any one of embodiments 1 to 30, wherein said additive solution is selected from the group consisting of AS7, AS7G-NAC, AS7G-NAC with 4 mM of gluconate (AS7GG-NAC), AS3 with gluconate, erythrosol-5, erythrosol-5G, erythrosol-5G with 5 mM Gluconate (erythrosol-5GG).

Embodiment 32. The method of any one of embodiments 1 to 31, wherein said blood product comprises 0.8% or less hemolysis after 42 days of said storing.

Embodiment 33. The method of any one of embodiments 1 to 32, wherein said blood product comprises whole blood, platelets, leukocytes, or red blood cells.

Embodiment 34. The method of any one of embodiments 1 to 33, wherein said BC carbon dioxide permeable bag comprises polyvinyl chloride (PVC) or polyolefin, silicone, polyvinylidene fluoride (PVDF), polysulphone (PS), polypropylene (PP) or polyurethane (PU).

Embodiment 35. A container for storing blood comprising a DEHP-free carbon dioxide permeable and oxygen impermeable material, wherein said material comprises a gas permeability for oxygen of less than 0.05 cm³/cm² at 1 atm at 25° C. and gas permeability for carbon dioxide of at least 0.62 centimeters cubed per centimeters squared (cm³/cm²) at 1 atm at 25° C.

Embodiment 36. The container of embodiment 35, wherein said material is selected from the group consisting of polyvinyl chloride (PVC), polyolefin, silicone, polyvinylidene fluoride (PVDF), polysulphone (PS), polypropylene (PP) or polyurethane.

Embodiment 37. The container of any one of embodiments 35 to 36, wherein said material comprises 1,2-Cyclohexane dicarboxylic acid diisononyl ester (DINCH) or butyryltrihexylcitrate (BTHC) as a plasticizer.

Embodiment 38. A method for treating a blood product comprising: adding an additive solution to said blood product; and storing said blood product in a DEHP-free blood compatible (BC) carbon dioxide permeable bag comprising a gas permeability for carbon dioxide of at least 0.62 cm³/cm² at about 1 atm at 25° C., wherein said storage is at least 7 days and said blood product comprises an oxygen level at said 7 days of storage that is decreased or about the same as an oxygen level in said blood product at day 1 of storage.

Embodiment 39. The method of embodiment 38, wherein said blood product comprises whole blood, platelets, leukocytes, or red blood cells.

Embodiment 40. The method of any one of embodiments 38 to 39, wherein said BC carbon dioxide permeable bag comprises PVC or polyolefin.

Embodiment 41. The method of embodiment 40, wherein said BC carbon dioxide permeable bag comprises between 20 and 70% weight/weight in PVC of 1,2-cyclohexane dicarboxylic acid diisononyl ester (DINCH) or butyryltrihexylcitrate (BTHC) as a plasticizer.

Embodiment 42. The method of embodiment 41, wherein said plasticizer is 20-45% w BTHC/weight in PVC.

Embodiment 43. The method of any one of embodiments 38 to 42, wherein said BC carbon dioxide permeable bag comprises a gas permeability for carbon dioxide of at least 2.0 cm³/cm² at about 1 atm at 25° C.

Embodiment 44. The method of any one of embodiments 38 to 43, wherein said BC carbon dioxide permeable bag further comprises an outer bag impermeable to oxygen and carbon dioxide.

Embodiment 45. The method of any one of embodiments 38 to 44, further comprising a carbon dioxide sorbent between said BC carbon dioxide permeable bag and said outer bag.

Embodiment 46. The method of embodiment 45, wherein said carbon dioxide sorbent further comprises an oxygen sorbent.

Embodiment 47. The method of any one of embodiments 38 to 46, wherein said BC carbon dioxide permeable bag comprises a gas permeability for oxygen of at least 0.05 cm³/cm²/atm 24 hrs. at 25° C.

Embodiment 48. The method of embodiment 47, wherein said BC carbon dioxide permeable bag comprises a gas permeability for oxygen of at least 0.15 cm³/cm²/atm 24 hrs. at 25° C.

Embodiment 49. The method of embodiment 48, wherein said BC carbon dioxide permeable bag comprises a gas permeability for oxygen of about 0.2 cm³/cm²/atm 24 hrs. at 25° C.

Embodiment 50. The method of any one of embodiments 38 to 49, wherein said blood product comprises greater than 15% SO2 after at least 7 days of storage.

Embodiment 51. The method of embodiment 50, wherein said blood product comprises greater than 20% SO2 after at least 7 days of storage.

Embodiment 52. The method of any one of embodiments 38 to 51, wherein said blood product comprises less than 125 mmHg pCO2.

Embodiment 53. The method of embodiment 52, wherein said blood product comprises less than 100 mmHg pCO2.

Embodiment 54. The method of embodiment 53, wherein said blood product comprises less than 75 mmHg pCO2.

Embodiment 55. The method of embodiment 54, wherein said blood product comprises less than 50 mmHg pCO2.

Embodiment 56. The method of any one of embodiments 38 to 55, wherein said storing is for at least 14 days.

Embodiment 57. The method of embodiment 56, wherein said storing is for at least 21 days.

Embodiment 58. The method of embodiment 57, wherein said storing is for at least 28 days.

Embodiment 59. The method of embodiment 58, wherein said storing is for at least 42 days.

Embodiment 60. The method of embodiment 59, wherein said storing is for at least 56 days.

Embodiment 61. The method of any one of embodiments 38 to 60, wherein said additive solution is selected from the group consisting of additive solution 7 (AS7), AS7G-NAC, AS7G-NAC with 4 mM of Gluconate (AS7GG-NAC), Erythrosol-5, Erythrosol-5G, Erythrosol-5G with 5 mM Gluconate.

Embodiment 62. The method of any one of embodiments 38 to 61, wherein said blood product comprises 0.8% or less hemolysis.

Embodiment 63. The method of any one of embodiments 38 to 62, wherein said blood product comprises whole blood, platelets, leukocytes, or red blood cells.

Embodiment 64. A method for storing a storable blood comprising: placing a blood product in a storage container comprising: a DEHP-free blood compatible (BC) material having a permeability to carbon dioxide of at least 0.62 cm³/cm² at about 1 atm at 25° C. and a permeability to oxygen of no more than 0.3 cm³/cm² at about 1 atm, and a carbon dioxide sorbent; and storing said container comprising said storable blood for a period to prepare stored blood.

Embodiment 65. The method of embodiment 64, wherein said storable blood comprises whole blood, platelets, leukocytes, or red blood cells.

Embodiment 66. The method of any one of embodiments 64 to 65, wherein said storable blood comprises 0.8% or less hemolysis after 42 days of storage.

Embodiment 67. The method of any one of embodiments 64 to 66, wherein said blood comprises 0.5% or less hemolysis after 42 days of storage.

Embodiment 68. The method of embodiment 66, wherein said blood comprises 0.5% or less hemolysis after 56 days of storage.

Embodiment 69. The method of embodiment 66, wherein said blood comprises 0.4% or less hemolysis after 56 days of storage.

Embodiment 70. The method of any one of embodiments 64 to 69, wherein said 2,3-DPG levels are increased at day 7, 21, 28, 35, 42 or 56 days of said storing in said blood product compared to 2,3-DPG levels of a conventionally stored blood product.

Embodiment 71. The method of embodiment 70, wherein said 2,3-DPG levels are increased by 10, 20, 30, 40, 50, 60, 70, or 80%.

Embodiment 72. The method of any one of embodiments 64 to 71, wherein said 2,3-DPG levels are increased up to 21 days of said storing in said blood product compared to 2,3-DPG levels of a conventionally stored blood product.

Embodiment 73. The method of any one of embodiments 64 to 72, wherein said 2,3-DPG levels are increased up to 28 days of said storing in said blood product compared to 2,3-DPG levels of a conventionally stored blood product.

Embodiment 74. The method of any one of embodiments 64 to 73, wherein said 2,3-DPG levels are increased up to 35 days of said storing in said blood product compared to 2,3-DPG levels of a conventionally stored blood product.

Embodiment 75. The method of any one of embodiments 64 to 74, wherein said 2,3-DPG levels are increased up to 42 days of said storing in said blood product compared to 2,3-DPG levels of a conventionally stored blood product.

Embodiment 76. The method of any one of embodiments 64 to 75, wherein said 2,3-DPG levels are increased up to 56 days of said storing in said blood product compared to 2,3-DPG levels of a conventionally stored blood product.

Embodiment 77. The method of any one of embodiments 64 to 76, wherein said ATP levels are increased compared to ATP levels of a conventionally stored blood product.

Embodiment 78. The method of any one of embodiments 64 to 77, wherein said ATP levels are increased compared to ATP levels of a conventionally stored blood product after 21 days of storage.

Embodiment 79. The method of any one of embodiments 64 to 78, wherein said ATP levels are increased compared to ATP levels of a conventionally stored blood product after 28 days of storage.

Embodiment 80. The method of any one of embodiments 64 to 79, wherein said ATP levels are increased compared to ATP levels of a conventionally stored blood product after 35 days of storage.

Embodiment 81. The method of any one of embodiments 64 to 80, wherein said ATP levels are increased compared to ATP levels of a conventionally stored blood product after 42 days of storage.

Embodiment 82. The method of any one of embodiments 64 to 81, wherein said ATP levels are increased compared to ATP levels of a conventionally stored blood product after 56 days of storage.

Embodiment 83. The method of any one of embodiments 64 to 82, wherein said BC material comprises 1,2-cyclohexane dicarboxylic acid diisononyl ester (DINCH) or butyryltrihexylcitrate BTHC as a plasticizer.

Embodiment 84. The method of embodiment 83, wherein said plasticizers is between 20 and 40%, 25 and 45%, 20 and 70%, and 40 and 70% weight/weight in PVC.

Embodiment 85. The method of any one of embodiments 64 to 84, wherein said storage container further comprises an oxygen sorbent between said BC material and an outer bag.

Embodiment 86. The method of any one of embodiments 64 to 85, further comprising adding an additive solution to said blood product and selected from the group consisting of AS7, AS7G-NAC, AS7G-NAC with 4 mM of Gluconate (AS7GG-NAC), Erythrosol-5, Erythrosol-5G, Erythrosol-5G with 5 mM Gluconate.

Embodiment 87. The method of any one of embodiments 64 to 86, wherein said BC material comprises polyvinyl chloride (PVC) or polyolefin.

Embodiment 88. The method of any one of embodiments 64 to 87, wherein said blood product comprises greater than 10% SO2 at day 1, 7, 14, 21, 42, or 56 days of storage.

Embodiment 89. The method of embodiment 61, wherein said blood product comprises greater than 20% SO2.

Embodiment 90. The method of any one of embodiments 64 to 89, said BC material comprises a permeability to carbon dioxide of at least 2 cm³/cm² at about 1 atm at 25° C.

Embodiment 91. The method of any one of embodiments 64 to 90, wherein said storage container further comprises an oxygen sorbent between said BC material and an outer bag.

Embodiment 92. A method for storing red blood cells comprising: placing said red blood cells in a storage container comprising: an outer oxygen and carbon dioxide impermeable container enclosing a DEHP-free blood compatible (BC) permeable inner collapsible container consisting of a material having a permeability to carbon dioxide of at least 0.62 cm³/cm² at about 1 atm and a permeability to oxygen of no more than 0.3 cm³/cm² at about 1 atm at 25° C. and enclosing a carbon dioxide sorbent, an oxygen sorbent, or an oxygen and a carbon dioxide sorbent between said inner and outer bag; and storing said container comprising said red blood cells for at least 7 days to prepare a stored blood product.

Embodiment 93. The method of embodiment 92, wherein said storing is at 4° C.

Embodiment 94. A method for maintaining the level of 2,3-DPG in a blood product comprising: placing a blood product comprising an oxygen saturation of at least 10% in a storage container comprising an outer oxygen and carbon dioxide impermeable container enclosing a blood compatible (BC) material having a permeability to carbon dioxide of at least 0.62 cm³/cm² at about 1 atm at 25° C. and a permeability to oxygen of no more than 0.3 cm³/cm² at about 1 atm and enclosing a carbon dioxide sorbent between said inner and outer bag, and storing said container comprising said blood product, wherein said level of 2,3-DPG is increased for up to 14 days of storage compared to a level of 2,3-DPG of a blood product conventionally stored.

Embodiment 95. The method of embodiment 94, wherein said 2,3-DPG is increased for up to 21 days of storage compared to a level of 2,3-DPG of a blood product conventionally stored.

Embodiment 96. The method of embodiment 94, wherein said BC material comprises PVC or polyolefin.

Embodiment 97. The method of embodiment 94, wherein said BC material comprises a DINCH or BTHC plasticizer.

Embodiment 98. A method for maintaining the level of ATP a blood product comprising: placing a blood product comprising an oxygen saturation of at least 10% in a storage container comprising an outer oxygen and carbon dioxide impermeable container enclosing a blood compatible (BC) material having a permeability to carbon dioxide of at least 0.62 cm³/cm² at about 1 atm at 25° C. and a permeability to oxygen of no more than 0.3 cm³/cm² at about 1 atm and enclosing a carbon dioxide sorbent between said inner and outer bag, and storing said container comprising said blood product, wherein said level of ATP is increased after 42 days of storage compared to a level of ATP of a blood product conventionally stored.

Embodiment 99. The method of embodiment 98, wherein said ATP is increased by at least 10% compared to the level of ATP of a blood product conventionally stored.

Embodiment 100. The method of embodiment 98, wherein said ATP is increased by at least 20% compared to the level of ATP of a blood product conventionally stored.

Embodiment 101. The method of embodiment 98, wherein said 2,3-DPG is increased for up to 21 days of storage compared to a level of 2,3-DPG of a blood product conventionally stored.

Embodiment 102. The method of embodiment 101, wherein said 2,3-DPG is increased by at least 10% compared to said blood product connotationally stored.

Embodiment 103. The method of embodiment 98, wherein said BC material comprises PVC or polyolefin.

Embodiment 104. The method of embodiment 98, wherein said BC material comprises a DINCH or BTHC plasticizer.

Embodiment 105. A composition comprising: a blood product selected from the group consisting of whole blood, platelets, and leukocytes; and an additive solution comprising sodium bicarbonate (NaHCO₃); sodium phosphate dibasic (Na₂HPO₄); adenine; guanosine; glucose; mannitol; N-acetyl-cysteine; 6-hydroxy-2,5,7,8-tetramethylchroman-2-carboxylic acid (Trolox); and 1-ascorbic acid (vitamin C).

Embodiment 106. The composition of embodiment 105, further comprising gluconate.

Embodiment 107. The composition of embodiment 105, wherein the concentration of said sodium bicarbonate is between 10 and 60 millimolar (mM).

Embodiment 108. The composition of embodiment 105, wherein the concentration of said Sodium phosphate dibasic (Na₂HPO₄) is between 10 and 20 mM.

Embodiment 109. The composition of embodiment 105, wherein the concentration of said Gluconate is between 0 and 10 mM.

Embodiment 110. The composition of embodiment 105, wherein the concentration of said Adenine is between 0 and 5 mM.

Embodiment 111. The composition of embodiment 105, wherein the concentration of said Guanosine is between 0 and 5 mM.

Embodiment 112. The composition of embodiment 105, wherein the concentration of said Glucose is between 50 and 100 mM.

Embodiment 113. The composition of embodiment 105, wherein the concentration of said Mannitol is between 40 and 80 mM.

Embodiment 114. The composition of embodiment 105, wherein the concentration of said N-Acetyl-Cysteine is between 0 and 1 mM.

Embodiment 115. The composition of embodiment 105, wherein the concentration of said Trolox is between 0 and 1 mM.

Embodiment 116. The composition of embodiment 105, wherein the concentration of said Vitamin C is between 0 and 1 mM.

Embodiment 117. The composition of embodiment 105, wherein said composition comprises a pH of between 6 and 7.

Embodiment 118. An additive composition comprising a concentration of: N-Acetyl-Cysteine; 6-Hydroxy-2,5,7,8-tetramethylchroman-2-carboxylic acid (Trolox); and 1-ascorbic acid, wherein said additive composition comprises a pH from 8 to 9.

Embodiment 119. The composition of embodiment 118, further comprising a concentration of: sodium bicarbonate (NaHCO₃); sodium phosphate dibasic (Na₂HPO₄); adenine; guanosine; glucose; and mannitol.

Embodiment 120. The composition of embodiment 118, further comprising gluconate.

Embodiment 121. The composition of embodiment 119, wherein the concentration of said sodium bicarbonate is between 10 and 60 millimolar (mM).

Embodiment 122. The composition of embodiment 121, wherein said concentration of said sodium bicarbonate (NaHCO₃) is between 20 and 50 mM.

Embodiment 123. The composition of embodiment 122, wherein said concentration of said Sodium bicarbonate (NaHCO₃) is between 25 and 45 mM.

Embodiment 124. The composition of embodiment 123, wherein said concentration of said sodium bicarbonate (NaHCO₃) is 26 mM.

Embodiment 125. The composition of embodiment 122, wherein said concentration of said sodium bicarbonate (NaHCO₃) is 40 mM.

Embodiment 126. The composition of embodiment 106, wherein said concentration of said sodium bicarbonate (NaHCO₃) is at least 25 mM.

Embodiment 127. The composition of embodiment 119, wherein the concentration of said sodium phosphate dibasic (Na₂HPO₄) is between 10 and 20 mM.

Embodiment 128. The composition of embodiment 119, wherein said concentration of said sodium phosphate dibasic (Na₂HPO₄) is at least 10 mM.

Embodiment 129. The composition of embodiment 119, wherein said concentration of said sodium phosphate dibasic (Na₂HPO₄) is 12 mM.

Embodiment 130. The composition of embodiment 120, wherein the concentration of said gluconate is between 0 and 10 mM.

Embodiment 131. The composition of embodiment 130, wherein said concentration of said gluconate is about 4 mM.

Embodiment 132. The composition of embodiment 119, wherein the concentration of said adenine is between 0 and 5 mM.

Embodiment 133. The composition of embodiment 132, wherein said concentration of said adenine is 2 mM.

Embodiment 134. The composition of embodiment 119, wherein the concentration of said guanosine is between 0 and 5 mM.

Embodiment 135. The composition of embodiment 119, wherein said concentration of said guanosine is between 1 and 2 mM.

Embodiment 136. The composition of embodiment 135, wherein said concentration of said guanosine is about 1.4 mM.

Embodiment 137. The composition of embodiment 119, wherein the concentration of said glucose is between 50 and 100 mM.

Embodiment 138. The composition of embodiment 137, wherein said concentration of said glucose is about 80 mM.

Embodiment 139. The composition of embodiment 119, wherein the concentration of said mannitol is between 40 and 80 mM.

Embodiment 140. The composition of embodiment 139, wherein said concentration of said mannitol is about 55 mM.

Embodiment 141. The composition of embodiment 118, wherein the concentration of said N-acetyl-cysteine is between 0 and 1 mM.

Embodiment 142. The composition of embodiment 141, wherein said concentration of said N-acetyl-cysteine is about 0.5 mM.

Embodiment 143. The composition of embodiment 118, wherein the concentration of said Trolox is between 0 and 1 mM.

Embodiment 144. The composition of embodiment 143, wherein said concentration of said 6-Hydroxy-2,5,7,8-tetramethylchroman-2-carboxylic acid (Trolox) is about 0.5 mM.

Embodiment 145. The composition of embodiment 118, wherein the concentration of said Vitamin C is between 0 and 1 mM.

Embodiment 146. The composition of embodiment 145, wherein said concentration of said Vitamin C is about 0.25 mM.

Embodiment 147. The composition of embodiment 118, wherein said composition comprises a pH of 8.75.

Embodiment 148. A composition comprising: a blood product selected from the group consisting of whole blood, platelets, and leukocytes; and an additive solution comprising a concentration of sodium phosphate dibasic (Na₂HPO₄); sodium citrate, adenine, guanosine, glucose, and mannitol.

Embodiment 149. The composition of embodiment 148, wherein said guanosine is at a concentration of between 1 and 2 mM.

Embodiment 150. The composition of embodiment 149, wherein said guanosine is 1.5 mM.

Embodiment 151. The composition of embodiment 148, further comprising gluconate at a concentration of between 2 and 8 mM.

Embodiment 152. The composition of embodiment 148, wherein said gluconate 5 mM.

Embodiment 153. The composition of embodiment 148, wherein the concentration of said sodium phosphate dibasic (Na₂HPO₄) is between 10 and 30 mM.

Embodiment 154. The composition of embodiment 148, wherein said concentration of said sodium phosphate dibasic (Na₂HPO₄) is at least 15 mM.

Embodiment 155. The composition of embodiment 148, wherein said concentration of said sodium phosphate dibasic (Na₂HPO₄) is 20 mM.

Embodiment 156. The composition of embodiment 148, wherein the concentration of said sodium citrate is between 10 and 30 mM.

Embodiment 157. The composition of embodiment 148, wherein said concentration of said sodium citrate is about 25 mM.

Embodiment 158. The composition of embodiment 148, wherein the concentration of said adenine is between 0 and 5 mM.

Embodiment 159. The composition of embodiment 148, wherein said concentration of said adenine is about 1.5 mM.

Embodiment 160. The composition of embodiment 148, wherein the concentration of said glucose is between 30 and 60 mM.

Embodiment 161. The composition of embodiment 160, wherein said concentration of said glucose is about 45.5 mM.

Embodiment 162. The composition of embodiment 148, wherein the concentration of said mannitol is between 80 and 140 mM.

Embodiment 163. The composition of embodiment 162, wherein said concentration of said mannitol is about 110 mM.

Embodiment 164. The composition of embodiment 148, wherein said composition comprises a pH of 8.8.

### EXAMPLES

### Example 1: Preparation and Storage of RBCs for Sampling

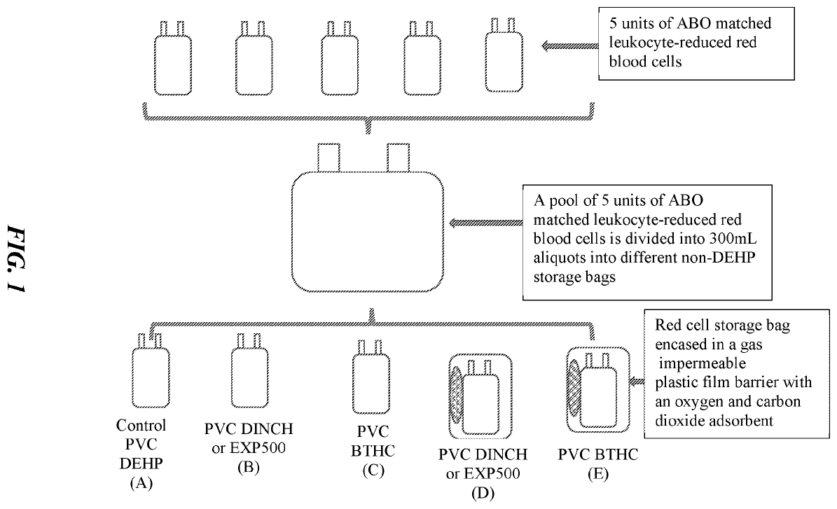

About 450 to 500 mL of whole blood is collected from healthy blood donors into a citrate phosphate double dextrose (CP2D) anticoagulant (Haemonetics, Braintree, Mass., Catalog number HAE PN 129-92 CP2D/AS3 set). Leukocyte-reduced packed RBCs (LR-pRBCs) are prepared from whole blood after leukocyte reduction and centrifugation at room temperature per the standard protocols at the Rhode Island Blood Center (RIBC). AS7G-NAC (Examples 1, 3, and 5) or AS3 (Example 4) additive solution is added to the LR-pRBCs to prepare LR-RBCs. See FIG. 1. For each test, 5 units of ABO matched LR-RBCs (300-350 mL each) in additive solution were pooled together into a 3-liter non-DEHP pooling bag. Equal aliquots (300 mL each) were transferred into either a blood bag having a single bag or a blood bag enclosed in a gas impermeable barrier (e.g., oxygen and carbon dioxide impermeable bag or overwrap) with an oxygen and carbon dioxide sorbent [Mitsubishi Gas Chemical Company, Tokyo, Japan; Mitsubishi SS-200 catalog number COM-600-0011; Desicare Inc., Mississippi, USA; Catalog number M1200BO3 disposed between the inner bag and the overwrap as provided in Table 5.

The blood storage bags are stored under ambient temperature (control; Bag A) or between 1 and 4° C. (Bags B to F) for up to 56 days in ambient air.

TABLE 5
Blood storage bags

| | | | Gas | | | |
|---|---|---|---|---|---|---|
| | | | Impermeable | | Gas permeability of inner bag | |
| Bag | Plastic | Plasticizer | Barrier | Sorbent | CO₂ | O₂ |
| A | PVC | DEHP | No | No | + | − |
| B | PVC | DEHP | Yes | Yes | + | − |
| C | PVC | DINCH | No | No | ++ | − |
| D | PVC | DINCH | Yes | Yes | ++ | − |
| E | PVC | BTHC | No | No | +++ | + |
| F | PVC | BTHC | Yes | Yes | +++ | + |
| G | Polyolefin | N/A | No | No | +++ | + |
| | (EXP 500) | | | | | |
| H | Polyolefin | N/A | Yes | Yes | +++ | + |
| | (EXP 500) | | | | | |

Bag A is a conventional storage bag. Bag B is an anaerobic storage bag essentially as described in U.S. Pat. No. 9,801,784.
− An oxygen or carbon dioxide permeability of less than 0.2
+ An oxygen or carbon dioxide permeability from 0.2 and 0.6
++ An oxygen or carbon dioxide permeability from 0.6 and 2.0
+++ An oxygen or carbon dioxide permeability of greater than 2.0

### Example 2: Storage of RBCs in an ASB Storage Bag with AS3 Storage Solution Maintains Higher Levels of Key Metabolites Compared to Conventional Storage

In this example, a unit of LR-RBC (300 mL) in AS3 was obtained from Rhode Island Blood Center (Rhode Island, US) and split into equal aliquots of 150 mL each into either a single standard PVC DEHP bag A configured for 150 mL volume or a similar blood bag B enclosed in a gas impermeable barrier (e.g., oxygen and carbon dioxide impermeable bag or overwrap) with an oxygen and carbon dioxide sorbent disposed between the inner bag and the overwrap.

Aliquots are collected from bags A and B at days 0, 7, 14, 21, 28, 35, and 42. The aliquots are analyzed for Blood gases, p50, pH, lactate, glucose (using an ABL90 gas analyzer with co-oximeter, Radiometer, Denmark), ATP, 2,3-DPG, and hemolysis. The data for p50 is calculated from the data from gas analyzer (ABL 90, Radiometer with co-oximeter) using a linear regression equation from p50 values measured with Hemox analyzer (TCS scientific, New Hope, Pa., USA) at pH 7.4, pCO₂ of 40 mmHg and temperature of 37° C. and compared to a calibration curve to convert p50 data from the ABL90 into Hemox analyzer values.

The collected data from replicate samples are analyzed by analysis of variance (ANOVA) using a Neuman-Keuls multiple comparison test, with a probability level of less than 0.05 being considered significant. The results are presented as the mean±standard error of the mean (SEM) or standard deviation (SD).

The percentage of saturated oxygen (% SO2) increased as a function of storage duration for RBCs that are stored in conventional bag A (Table 6). In contrast, the levels remain constant with minor decrease in RBCs that are stored in ASB bag B with 02/CO2 impermeable barrier (Table 7). The pCO₂ initially increases up to 28 days followed by a gradual decrease during 28 to 42 days of storage for both RBCs that are stored in storage bags A and B. The pCO₂ levels in conventionally stored RBCs are significantly higher than RBCs in ASB storage bags at all measured days during the storage period, p<0.0001. The results of hemolysis in RBCs in conventional and ASB storage bags are also summarized in Table 6 and Table 7. There are no significant differences in hemolysis between conventional and Hemanext storage conditions during the storage period (p>0.05).

Storage of RBCs in bag B (Table 7) results in significantly higher ATP concentrations when compared to conventional storage on days 28, 35 and 42 of storage (p<0.005; Table 6). ASB bag B storage conditions result in significantly higher 2,3-DPG concentrations at storage days 7 and 14 compared to conventionally stored blood (p<0.05). The 2,3-DPG concentration rapidly declines during storage such that by day 21, the levels are at the limit of detection of the assay at 0.25 μmol/gHb. RBCs stored in bag B also show a significant increase in lactate and p50 levels during the storage period compared to conventionally stored RBCs (lactate p<0.0001 for all data points; p50 p<0.001 at time points 7 to 42). Further, pH levels remain significantly higher in RBCs stored in the ASB storage bag (B) compared to conventionally stored RBCs in bag A. Importantly, 2,3-DPG levels did not correlate with pH. For example, the pH changes from 6.631±0.075 to 6.279±0.052 with conventional storage, and from 6.638±0.076 to 6.329±0.054 for Hemanext storage.

The data p50 is calculated using the data from gas analyzer (ABL 90, Radiometer with co-oximeter) using a linear regression equation from p50 values measured with Hemox analyzer (TCS scientific, New Hope, Pa., USA) at pH 7.4, pCO2 of 40 mmHg and temperature of 37° C.

TABLE 6
Blood Characteristics in PVC + DEHP (Bag A with AS3 storage solution)

| | PVC w DEHP | | | | | | |
|---|---|---|---|---|---|---|---|
| | (n = 34) | | | | | | |
| Day | 1 | 7 | 14 | 21 | 28 | 35 | 42 |
| In Vitro Metrics | | | | | | | |
| % SO₂ | 43.8 ± 17.9 | 49.8 ± 17.2 | 56.9 ± 15.8 | 63.9 ± 14.9 | 70.0 ± 13.5 | 76.3 ± 12.4 | 82.1 ± 11.2 |
| CO₂ (mmHg) | 95.6 ± 10.5 | 108.5 ± 9.0 | 115.0 ± 8.6 | 116.1 ± 9.1 | 113.8 ± 11.0 | 108.9 ± 11.3 | 102.5 ± 10.2 |
| ATP (μmol/g Hb) | 4.8 ± 0.8 | 4.8 ± 0.6 | 3.5 ± 0.8 | 3.5 ± 0.8 | 3.3 ± 0.8 | 2.9 ± 0.7 | 2.6 ± 0.8 |
| 2,3DPG (μmol/g Hb) | 3.7 ± 2.8 | 1.4 ± 1.4 | 0.6 ± 0.6 | 0.5 ± 0.3 | 0.5 ± 0.3 | 0.6 ± 0.6 | 0.6 ± 0.6 |
| Lactate (mmol/L) | 5.4 ± 2.5 | 9.0 ± 2.6 | 12.4 ± 2.7 | 15.4 ± 2.8 | 17.9 ± 3.1 | 19.8 ± 3.1 | 21.6 ± 3.2 |
| *Hemolysis (%) | 0.07 ± 0.05 | 0.09 ± 0.07 | 0.13 ± 0.08 | 0.14 ± 0.09 | 0.17 ± 0.11 | 0.18 ± 0.14 | 0.20 ± 0.14 |
| Methemoglobin (%) | 0.42 ± 0.10 | 0.47 ± 0.09 | 0.50 ± 0.10 | 0.53 ± 0.11 | 0.60 ± 0.12 | 0.61 ± 0.13 | 0.64 ± 0.14 |
| p50 (mmHg) | 23.0 ± 1.1 | 22.7 ± 1.1 | 22.1 ± 1.2 | 21.1 ± 1.3 | 20.7 ± 1.3 | 19.7 ± 1.5 | 18.9 ± 1.7 |
| pH | 6.63 ± 0.06 | 6.53 ± 0.08 | 6.48 ± 0.04 | 6.42 ± 0.05 | 6.38 ± 0.04 | 6.33 ± 0.05 | 6.29 ± 0.05 |
| N | 34 | 34 | 34 | 34 | 34 | 34 | 34 |
| *N | 33 | 33 | 33 | 33 | 33 | 28 | 29 |

TABLE 7
Blood Characteristics in PVC + DEHP + Barrier (Bag B with AS3 storage solution)

| | ASB (PVC/ | | | | | | |
|---|---|---|---|---|---|---|---|
| | DEHP & Barrier) | | | | | | |
| | (n = 34) | | | | | | |
| Day | 1 | 7 | 14 | 21 | 28 | 35 | 42 |
| In Vitro Metrics | | | | | | | |
| % SO₂ | 41.8 ± 18.4 | 38.9 ± 17.3 | 37.6 ± 16.7 | 35.5 ± 16.0 | 34.0 ± 15.2 | 32.9 ± 14.3 | 31.6 ± 14.4 |
| CO₂ (mmHg) | 93.5 ± 11.3 | 103.9 ± 9.1 | 107.4 ± 9.0 | 104.6 ± 10.1 | 99.9 ± 11.2 | 91.8 ± 10.7 | 82.6 ± 10.2 |
| ATP (μmol/g Hb) | 4.8 ± 0.9 | 4.8 ± 0.7 | 4.5 ± 1.0 | 3.6 ± 0.8 | 3.3 ± 0.8 | 3.0 ± 0.8 | 2.7 ± 0.8 |
| 2,3DPG (μmol/g Hb) | 3.8 ± 2.8 | 1.6 ± 1.5 | 0.8 ± 0.7 | 0.5 ± 0.4 | 0.5 ± 0.4 | 0.6 ± 0.6 | 0.5 ± 0.7 |
| Lactate (mmol/L) | 6.3 ± 2.7 | 10.2 ± 3.1 | 13.4 ± 2.9 | 16.0 ± 3.0 | 19.0 ± 3.4 | 20.7 ± 3.2 | 22.3 ± 3.1 |
| *Hemolysis (%) | 0.07 ± 0.04 | 0.09 ± 0.07 | 0.13 ± 0.08 | 0.14 ± 0.09 | 0.17 ± 0.11 | 0.19 ± 0.14 | 0.20 ± 0.14 |
| Methemoglobin (%) | 0.43 ± 0.09 | 0.47 ± 0.08 | 0.51 ± 0.11 | 0.56 ± 0.11 | 0.60 ± 0.10 | 0.58 ± 0.13 | 0.62 ± 0.11 |
| p50 (mmHg) | 23.2 ± 1.1 | 23.1 ± 1.0 | 22.7 ± 1.1 | 22.2 ± 0.9 | 22.1 ± 0.9 | 21.9 ± 0.9 | 21.7 ± 0.9 |
| pH | 6.64 ± 0.06 | 6.54 ± 0.08 | 6.50 ± 0.05 | 6.45 ± 0.04 | 6.41 ± 0.04 | 6.38 ± 0.05 | 6.34 ± 0.05 |
| N | 34 | 34 | 34 | 34 | 34 | 34 | 34 |
| *N | 33 | 33 | 33 | 33 | 33 | 28 | 29 |

### Example 3: Storage of RBCs in Bags with Increased Permeability to CO₂ Maintains Higher Levels of Key Metabolites Compared to Conventional Storage

The role of DEHP and permeability is tested by comparing the results of bags with DEHP (bag A) and without DEHP (bags C, D, E, F). See Table 8

TABLE 8
Oxygen, carbon dioxide, hemolysis, and ATP levels at Day 21

| | Bag | | | | | |
|---|---|---|---|---|---|---|
| | | | C: PVC | D: | E: PVC w | F: DINCH + |
| | A: PVC/DEHP | | w BTHC | BTHC + barrier | DINCH | barrier |
| | Day | | | | | |
| | 0 | 21 | 21 | 21 | 21 | 21 |
| O₂ | 46.2 ± 5.3 | 67.8 ± 7.7 | 94.1 ± 5.4 | 40.2 ± 5.7 | 86.4 ± 7.9 | 40.0 ± 6.4 |
| (% SO₂) | | | | | | |
| CO₂ | 109.9 ± 21.5 | 149.5 ± 24.8 | 69.8 ± 12.8 | 73.4 ± 14.8 | 111.3 ± 16.2 | 108.9 ± 17.5 |
| (mmHg) | | | | | | |
| Hemolysis | 0.08 ± 0.03 | 0.15 ± 0.06 | 0.17 ± 0.05 | 0.17 ± 0.05 | 0.16 ± 0.05 | 0.18 ± 0.06 |
| (%) | | | | | | |
| ATP | 3.9 ± 0.4 | 3.9 ± 0.4 | 3.8 ± 0.5 | 4.3 ± 0.6 | 3.7 ± 0.5 | 4.3 ± 0.6 |

Data are the means ± standard deviations of 10 independent pools of red cell concentrates in alkaline based storage solutions (n = 10).

The % SO2 in RBCs increases over 42 days of storage in conventional storage bag A and bags C and E. Addition of an oxygen and carbon dioxide impermeable overwrap to bags D and F result in a maintenance or decrease in % SO₂ compared to day 1. Similarly, the pCO₂ increases at day 42 of storage in conventional bag A compared to the day 0 value (p<0.05). In contrast to RBCs conventionally stored, pCO₂ decreases significantly when RBCs are stored in high CO₂ permeability storage bags with and without an overwrap at 42 days

Not to be limited by theory, the data show that by maintaining a constant or decreasing level of O₂ while depleting the level of CO₂, ATP remains unchanged compared to conventional bags containing DEHP. Adding a barrier bag to the PVC with BTHC (bag C) and PVC with DINCH (bag E) to decrease the level of O₂ to below 30%, significantly increased the level of ATP by 12 and 14%, respectively. For example, the concentrations of ATP are significantly higher in DINCH (Bag D) and BTHC (Bag E) bags with gas impermeable barriers when compared to either the control DEHP or BTHC and DINCH bags without the barrier. See FIG. 2B and Table 9.

TABLE 9
Oxygen, carbon dioxide, hemolysis, and ATP levels at Day 21

| | Bag | | | | | |
|---|---|---|---|---|---|---|
| | A: PVC/DEHP | | C: PVC w BTHC | D: BTHC + barrier | E: PVC w DINCH | F: DINCH + barrier |
| Day | 0 | 42 | 42 | 42 | 42 | 42 |
| O₂ (% SO₂) | 46.2 ± 5.3 | 71.2 ± 26.8 | 98.0 ± 0.6 | 28.9 ± 9.3 | 96.3 ± 3.2 | 28.4 ± 12.9 |
| CO₂ (mmHg) | 109.9 ± 21.5 | 155.5 ± 28.2 | 39.6 ± 8.5 | 43.2 ± 12.2 | 80.0 ± 21.5 | 84.2 ± 29.2 |
| Hemolysis (%) | 0.08 ± 0.03 | 0.23 ± 0.05 | 0.35 ± 0.16 | 0.32 ± 0.09 | 0.29 ± 0.07 | 0.31 ± 0.08 |
| ATP at day 42 | 3.9 ± 0.4 | 3.2 ± 0.5 | 3.5 ± 0.4 | 4.5 ± 0.8 | 3.2 ± 0.4 | 4.2 ± 0.7 |

Surprisingly, maintaining the level of O₂ while depleting CO₂ by placing blood in a high CO₂ permeability bag (bags C and D) results in a significant increase in the level of 2,3-DPG compared to conventional storage. The concentrations of 2,3-DPG in RBCs that are stored in DEHP bags (Bag A) rapidly decline by about 79% on day 21 of storage when compared to the starting level (FIG. 3). The levels of 2,3-DPG in both BTHC and DINCH are higher than conventional stored RBCs after 21 days. The levels of 2,3-DPG are increased by approximately 20% with gas impermeable barriers in both BTHC and DINCH after 21 days as compared to BTHC and DINCH bags without barrier. Importantly, the effect of CO₂ on 2,3-DPG levels does not appear to be an effect of pH, but rather is closely correlated with CO₂ levels. This is surprising, as much of the literature has focused on the pH as a causative factor.

Importantly, hemolysis remained below the maximum safe levels of 1% and 0.8% established by U.S. and European regulatory authorities, respectively, whether DEHP was present or not (Table 10, Table 11, and Table 12).

In summary, the present data shows that depletion of CO₂ and the prevention of O₂ increases (either through depletion or maintenance) during storage is demonstrated as an improved method for reducing deleterious effects of various storage lesions. Thus, a storage system incorporating at least two features increases the ability to preserve the quality of the RBCs during refrigerated storage. First, a storage bag that prevents increases in O₂ levels in RBCs, by maintaining or decreasing the starting O₂ content, during prolonged storage at 1-6° C. helps maintain ATP levels. This maintenance or decrease in O₂ levels can be conveniently achieved through the selection of polymers that provide selective permeability of the polymer and optionally, the presence of O₂/CO₂ adsorbent and an outer-wrap or polymer that is impermeable to both CO₂ and O₂. Second, the storage bag also maintains a low level of CO₂ during storage which results in increased levels of 2,3-DPG and surprisingly maintaining 2,3-DPG at or near pre-storage levels.

TABLE 10
Blood Characteristics in PVC DEHP storage bags
(Bag A with AS7G-NAC solution, control)

| | PVC DEHP | | | |
|---|---|---|---|---|
| | Day | | | |
| In Vitro Metrics | 0 | 21 | 42 | 56 |
| % SO₂ | 46.2 ± 5.3 | 67.8 ± 7.7 | 71.2 ± 26.8 | 88.4 ± 10.6 |
| CO₂ (mmHg) | 109.9 ± 21.5 | 149.5 ± 24.8 | 155.5 ± 28.2 | 148.3 ± 22.3 |
| ATP (μmol/g Hb) | 3.9 ± 0.4 | 3.9 ± 0.4 | 3.2 ± 0.5 | 2.3 ± 0.5 |
| 2,3DPG (μmol/g Hb) | 11.6 ± 1.0 | 3.0 ± 2.2 | 1.9 ± 2.2 | 1.3 ± 0.7 |
| Lactate (mmol/L) | 3.4 ± 0.6 | 15.8 ± 1.0 | 25.2 ± 3.1 | 31.0 ± 4.1 |
| Hemolysis (%) | 0.08 ± 0.03 | 0.15 ± 0.06 | 0.23 ± 0.05 | 0.30 ± 0.08 |
| Methemoglobin (%) | 0.57 ± 0.07 | 0.77 ± 0.08 | 0.95 ± 0.10 | 1.04 ± 0.19 |
| pH | 6.94 ± 0.13 | 6.77 ± 0.10 | 6.56 ± 0.11 | 6.53 ± 0.06 |
| N | 10 | 10 | 10 | 8 |

TABLE 11
Blood Characteristics in DINCH storage bags (Bag C and Bag D with AS7G-NAC solution)

| | | DINCH (Bag C) | | | DINCH + Barrier (Bag D) | | |
|---|---|---|---|---|---|---|---|
| Day | 0 | 21 | 42 | 56 | 21 | 42 | 56 |
| In Vitro Metrics | | | | | | | |
| % SO₂ | 46.2 ± 5.3 | 86.4 ± 7.9 | 96.3 ± 3.2 | 97.6 ± 0.8 | 40.0 ± 6.4 | 28.4 ± 12.9 | 25.2 ± 11.9 |
| CO₂ (mmHg) | 109.9 ± 21.5 | 111.3 ± 16.2 | 80.0 ± 21.5 | 89.3 ± 43.2 | 108.9 ± 17.5 | 84.2 ± 29.2 | 77.1 ± 30.0 |
| ATP (μmol/g Hb) | 3.9 ± 0.4 | 3.7 ± 0.5 | 3.2 ± 0.4 | 2.6 ± 0.5 | 4.3 ± 0.6 | 4.2 ± 0.7 | 3.8 ± 1.3 |
| 2,3DPG (μmol/g Hb) | 11.6 ± 1.0 | 4.7 ± 4.4 | 2.9 ± 4.0 | 1.9 ± 0.7 | 5.8 ± 4.7 | 3.3 ± 3.8 | 2.0 ± 0.9 |
| Lactate (mmol/L) | 3.4 ± 0.6 | 16.4 ± 1.1 | 29.0 ± 2.9 | 34.5 ± 4.8 | 17.0 ± 1.4 | 27.2 ± 3.2 | 33.9 ± 4.9 |
| Hemolysis (%) | 0.08 ± 0.03 | 0.16 ± 0.05 | 0.29 ± 0.07 | 0.43 ± 0.11 | 0.18 ± 0.06 | 0.31 ± 0.08 | 0.43 ± 0.14 |
| Methemoglobin (%) | 0.57 ± 0.07 | 0.71 ± 0.13 | 1.00 ± 0.11 | 0.98 ± 0.29 | 0.74 ± 0.13 | 0.76 ± 0.17 | 0.78 ± 0.29 |
| pH | 6.94 ± 0.13 | 6.81 ± 0.13 | 6.58 ± 0.12 | 6.49 ± 0.12 | 6.83 ± 0.12 | 6.59 ± 0.12 | 6.43 ± 0.27 |
| N | 10 | 10 | 10 | 8 | 10 | 10 | 8 |

TABLE 12
Blood Characteristics in BTHC storage bags (Bag E and Bag F with AS7G-NAC solution)

| | | PVC + BTHC (Bag E) | | | PVC + BTHC + Barrier (Bag F) | | |
|---|---|---|---|---|---|---|---|
| Day | 0 | 21 | 42 | 56 | 21 | 42 | 56 |
| In Vitro Metrics | | | | | | | |
| % SO₂ | 46.2 ± 5.3 | 94.1 ± 5.4 | 98.0 ± 0.6 | 97.6 ± 0.8 | 40.2 ± 5.7 | 28.9 ± 9.3 | 23.0 ± 11.5 |
| CO₂ (mmHg) | 109.9 ± 21.5 | 69.8 ± 12.8 | 39.6 ± 8.5 | 26.3 ± 7.1 | 73.4 ± 14.8 | 43.2 ± 12.2 | 27.2 ± 8.0 |
| ATP (μmol/g Hb) | 3.9 ± 0.4 | 3.8 ± 0.5 | 3.5 ± 0.4 | 2.8 ± 0.6 | 4.3 ± 0.6 | 4.5 ± 0.8 | 3.8 ± 0.8 |
| 2,3DPG (μmol/g Hb) | 11.6 ± 1.0 | 8.1 ± 4.8 | 5.4 ± 2.8 | 4.1 ± 2.2 | 10.0 ± 4.0 | 5.4 ± 2.8 | 4.6 ± 2.4 |
| Lactate (mmol/L) | 3.4 ± 0.6 | 17.8 ± 1.6 | 32.0 ± 3.2 | 40.2 ± 2.3 | 18.5 ± 1.6 | 31.1 ± 4.3 | 39.1 ± 4.1 |
| Hemolysis (%) | 0.08 ± 0.03 | 0.17 ± 0.05 | 0.35 ± 0.16 | 0.53 ± 0.02 | 0.17 ± 0.05 | 0.32 ± 0.09 | 0.47 ± 0.18 |
| Methemoglobin (%) | 0.57 ± 0.07 | 0.66 ± 0.08 | 0.90 ± 0.13 | 0.98 ± 0.18 | 0.77 ± 0.10 | 0.75 ± 0.12 | 0.80 ± 0.28 |
| pH | 6.94 ± 0.13 | 6.89 ± 0.14 | 6.61 ± 0.14 | 6.49 ± 0.07 | 6.90 ± 0.12 | 6.62 ± 0.12 | 6.51 ± 0.07 |
| N | 10 | 10 | 10 | 8 | 10 | 10 | 8 |

### Example 4: RBCs in AS3 Additive Solution Stored in Bags with Increased Gas Permeability

The studies of Example 3 are repeated with AS3 to determine whether improvements in metabolites (e.g., ATP and 2,3-DPG) observed in various non-DEHP bags as provided above remain steady when using AS3 as the additive solution.

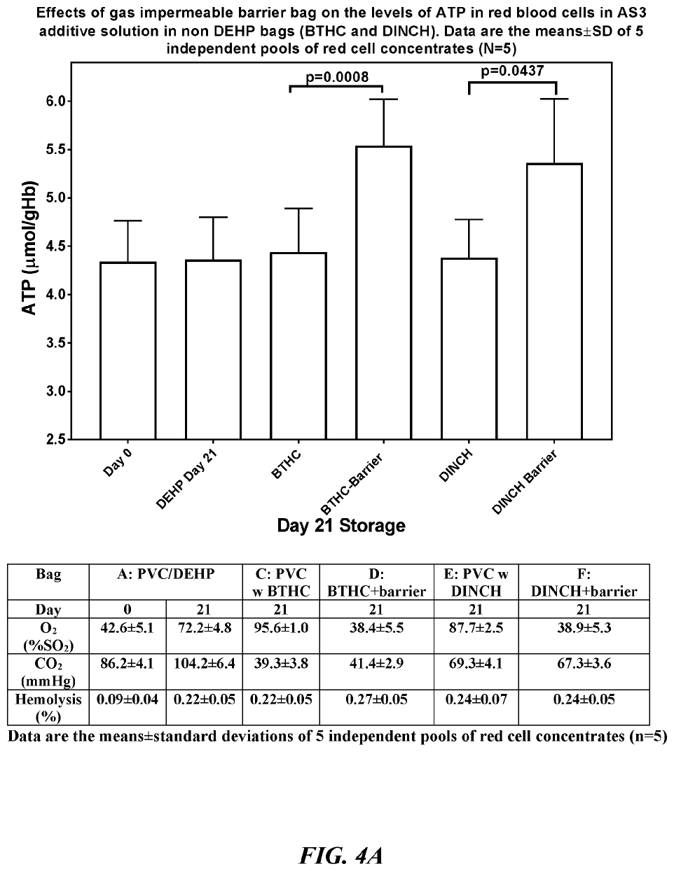

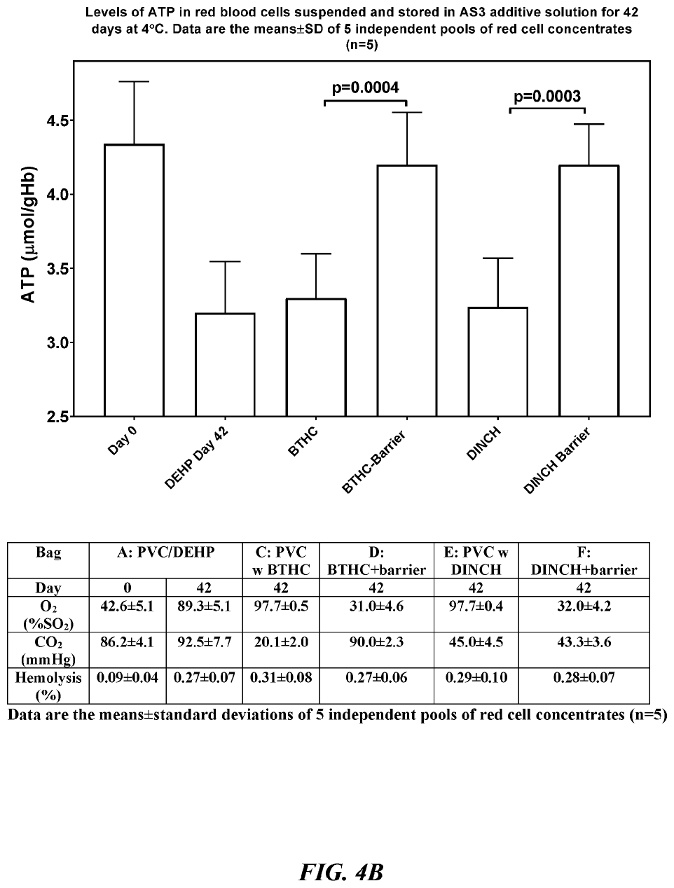

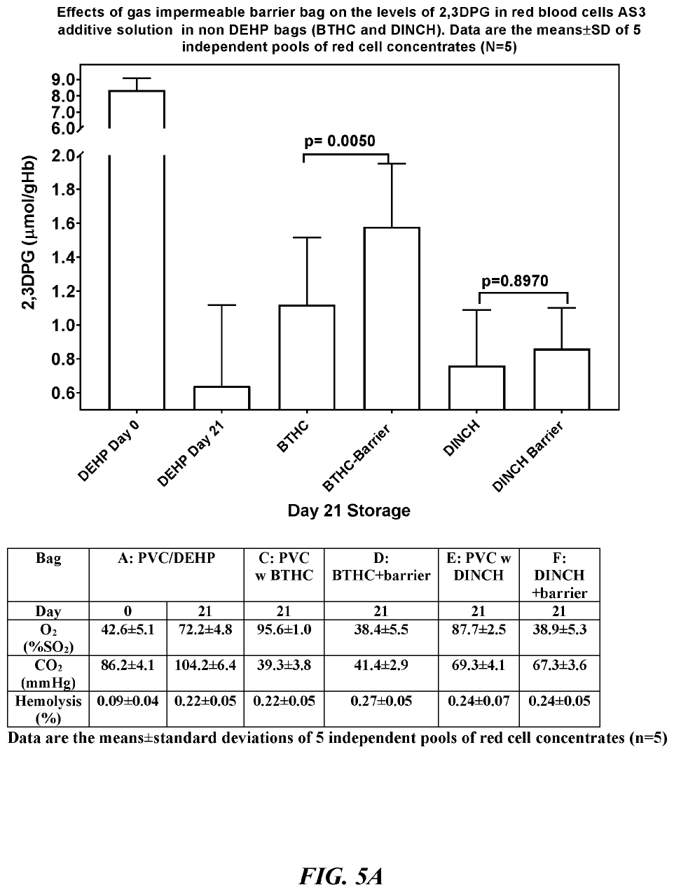

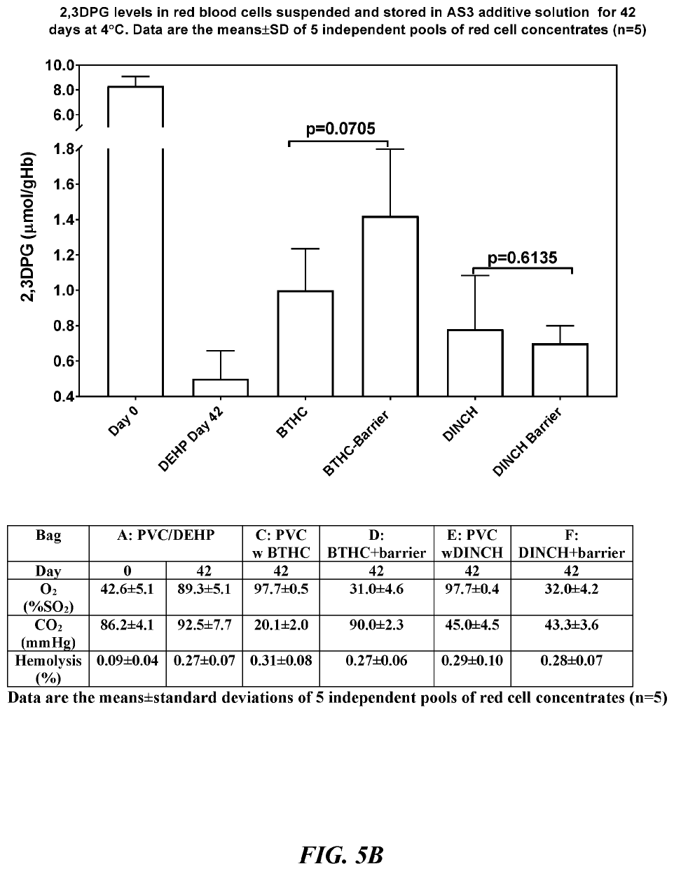

As seen with AS7G-NAC (FIGS. 2A, 2B, 3A, and 3B), the % SO2 levels are significantly decreased after 42 days in the DINCH and BTHC bags with the gas impermeable barrier bag compared to DEHP, DINCH, and BTHC bags without the barrier bag. Unlike the oxygen levels, the pCO2 levels remain similar in each non-DEHP bag selection (BTHC or DINCH) with and without the gas impermeable outer barrier bag. Both DINCH and BTHC show decreased pCO2 levels compared to DEHP after 21 days of storage (FIGS. 4 and 5).

Similar to the results with AS7G-NAG additive solution, ATP and 2,3-DPG levels increase in RBCs that are stored in DINCH and BTHC inner bags with the gas impermeable barrier bag compared to DINCH and BTHC bags without the outer bag and conventionally stored RBCs (FIG. 4 and FIG. 5). All RBC samples also remained below the required hemolysis cut-off Importantly, hemolysis remained below the maximum safe levels of 1% and 0.8% established by U.S and European regulatory authorities, respectively, whether DEHP was present or not (Table 13, Table 14 and Table 15)

TABLE 13
Blood Characteristics in PVC DEHP storage
bags (Bag A with AS3 solution, control)

| | PVC DEHP | | | |
|---|---|---|---|---|
| | Day | | | |
| In Vitro Metrics | 0 | 21 | 42 | 56 |
| % SO₂ | 42.6 ± 5.1 | 72.2 ± 4.8 | 89.3 ± 5.1 | 94.9 ± 3.8 |
| CO₂ (mmHg) | 86.2 ± 4.1 | 104.2 ± 6.4 | 92.5 ± 7.7 | 74.3 ± 2.1 |
| ATP (μmol/g Hb) | 4.3 ± 0.4 | 4.4 ± 0.4 | 3.2 ± 0.4 | 2.6 ± 0.7 |
| 2,3DPG (μmol/g Hb) | 8.3 ± 0.7 | 0.6 ± 0.5 | 0.5 ± 0.2 | 0.6 ± 0.1 |
| Lactate (mmol/L) | 2.7 ± 0.5 | 12.8 ± 0.4 | 20.6 ± 0.7 | 24.2 ± 0.1 |
| Hemolysis (%) | 0.09 ± 0.04 | 0.22 ± 0.05 | 0.27 ± 0.07 | 0.38 ± 0.14 |
| Methemoglobin (%) | 0.74 ± 0.21 | 0.85 ± 0.14 | 1.06 ± 0.17 | 1.00 ± 0.00 |
| pH | 6.80 ± 0.07 | 6.62 ± 0.14 | 6.40 ± 0.06 | 6.33 ± 0.01 |
| N | 5 | 5 | 5 | 2 |

TABLE 14
Blood Characteristics in DINCH storage bags (Bag C and Bag D with AS3 solution)

| | | DINCH (Bag C) | | | DINCH + Barrier (Bag D) | | |
|---|---|---|---|---|---|---|---|
| Day | 0 | 21 | 42 | 56 | 21 | 42 | 56 |
| In Vitro Metrics | | | | | | | |
| % SO₂ | 42.6 ± 5.1 | 87.7 ± 2.5 | 97.7 ± 0.4 | 98.0 ± 0.0 | 38.9 ± 5.3 | 32.0 ± 0.4 | 26.7 ± 6.6 |
| CO₂ (mmHg) | 86.2 ± 4.1 | 69.3 ± 4.1 | 45.0 ± 4.5 | 32.3 ± 7.0 | 67.3 ± 3.6 | 43.3 ± 3.6 | 28.9 ± 1.5 |
| ATP (μmol/g Hb) | 4.3 ± 0.4 | 4.4 ± 0.4 | 3.2 ± 0.3 | 2.4 ± 0.1 | 5.4 ± 0.7 | 4.2 ± 0.3 | 3.4 ± 0.2 |
| 2,3DPG (μmol/g Hb) | 8.3 ± 0.7 | 0.8 ± 0.3 | 0.8 ± 0.3 | 0.6 ± 0.0 | 0.9 ± 0.2 | 0.7 ± 0.1 | 0.6 ± 0.0 |
| Lactate (mmol/L) | 2.7 ± 0.5 | 13.8 ± 0.8 | 22.9 ± 0.8 | 26.6 ± 1.6 | 14.3 ± 1.0 | 22.7 ± 1.4 | 25.5 ± 1.6 |
| Hemolysis (%) | 0.09 ± 0.04 | 0.24 ± 0.07 | 0.29 ± 0.10 | 0.39 ± 0.07 | 0.24 ± 0.05 | 0.28 ± 0.07 | 0.38 ± 0.08 |
| Methemoglobin (%) | 0.74 ± 0.21 | 0.90 ± 0.20 | 1.08 ± 0.18 | 1.05 ± 0.07 | 0.76 ± 0.15 | 0.78 ± 0.05 | 0.90 ± 0.00 |
| pH | 6.80 ± 0.07 | 6.67 ± 0.20 | 6.40 ± 0.06 | 6.31 ± 0.02 | 6.70 ± 0.19 | 6.45 ± 0.06 | 6.37 ± 0.01 |
| N | 5 | 5 | 5 | 2 | 5 | 5 | 2 |

TABLE 15
Blood Characteristics in BTHC storage bags (Bag E and Bag F with AS3 solution)

| | | PVC + BTHC (Bag E) | | | PVC + BTHC + Barrier (Bag F) | | |
|---|---|---|---|---|---|---|---|
| Day | 0 | 21 | 42 | 56 | 21 | 42 | 56 |
| In Vitro Metrics | | | | | | | |
| % SO₂ | 42.6 ± 5.1 | 95.6 ± 1.0 | 97.7 ± 0.5 | 98.0 ± 0.0 | 38.4 ± 5.5 | 31.0 ± 4.6 | 26.0 ± 8.0 |
| CO₂ (mmHg) | 86.2 ± 4.1 | 39.3 ± 3.8 | 20.1 ± 2.0 | 15.9 ± 0.6 | 41.4 ± 2.9 | 19.0 ± 2.3 | 11.9 ± 0.6 |
| ATP (μmol/g Hb) | 4.3 ± 0.4 | 4.4 ± 0.5 | 3.3 ± 0.4 | 2.3 ± 0.3 | 5.5 ± 0.5 | 4.2 ± 0.4 | 3.2 ± 0.2 |
| 2,3DPG (μmol/g Hb) | 8.3 ± 0.7 | 1.1 ± 0.4 | 1.0 ± 0.2 | 0.9 ± 0.1 | 1.6 ± 0.7 | 1.4 ± 0.4 | 1.1 ± 0.1 |
| Lactate (mmol/L) | 2.7 ± 0.5 | 15.1 ± 0.8 | 25.0 ± 0.8 | 28.4 ± 0.3 | 15.6 ± 1.0 | 24.8 ± 1.5 | 27.7 ± 2.1 |
| Hemolysis (%) | 0.09 ± 0.04 | 0.22 ± 0.05 | 0.31 ± 0.08 | 0.41 ± 0.06 | 0.27 ± 0.06 | 0.30 ± 0.08 | 0.40 ± 0.05 |
| Methemoglobin (%) | 0.74 ± 0.21 | 0.86 ± 0.18 | 1.10 ± 0.20 | 1.15 ± 0.07 | 0.76 ± 0.17 | 0.78 ± 0.05 | 1.00 ± 0.00 |
| pH | 6.80 ± 0.07 | 6.68 ± 0.19 | 6.39 ± 0.06 | 6.30 ± 0.01 | 6.71 ± 0.17 | 6.43 ± 0.06 | 6.35 ± 0.01 |
| N | 5 | 5 | 5 | 2 | 5 | 5 | 2 |

### Example 5: RBCs in AS7G-NAC Additive Solution Stored in Bags with Increased Gas Permeability

Red blood cells are prepared with AS7G-NAC additive solution as provided in Examples 1 and 2. The RBCs are then placed into one of the following bags:
A. Conventional—PVC with DEHP
C. PVC with BTHC
D. PVC with BTHC inner bag with a CO₂/O₂ impermeable outer barrier
F. Polyolefin
G. Polyolefin inner bag with CO₂/O₂ impermeable outer barrier

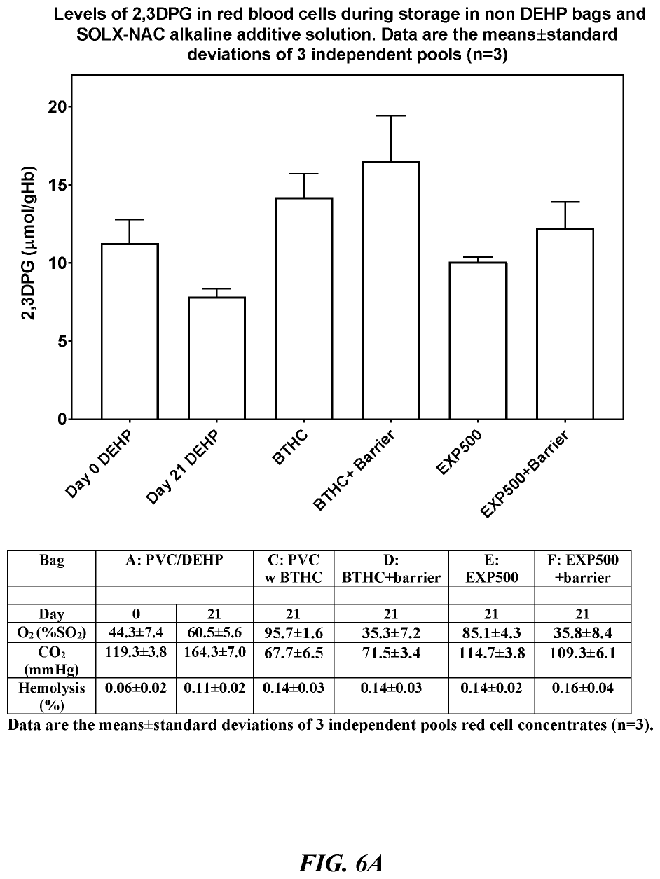

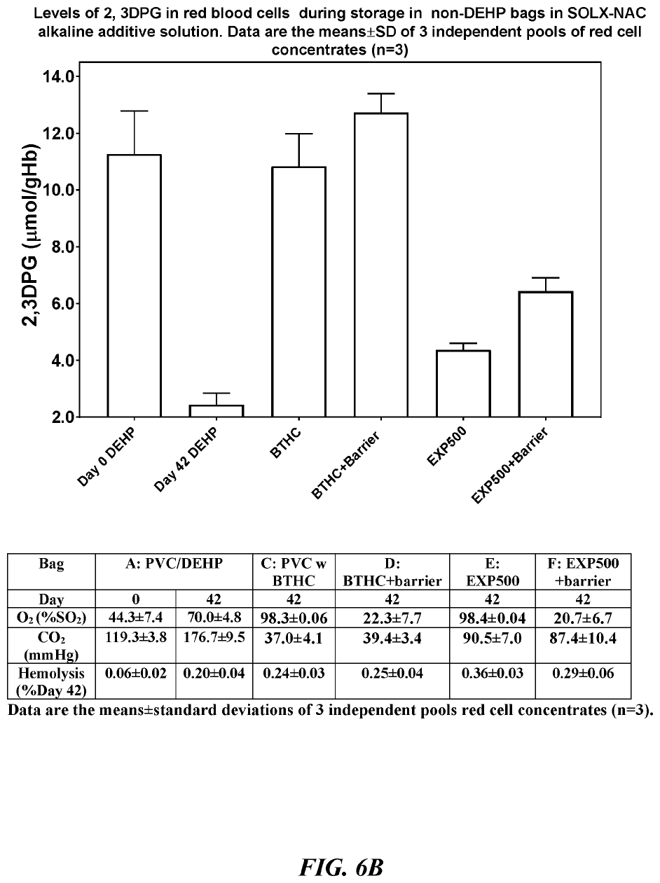

The concentrations of 2,3-DPG in RBCs that are stored in DEHP bags decrease by day 21 of storage when compared to the starting level (FIG. 6). The levels of 2,3-DPG in both BTHC (bag C) and polyolefin (EXP 500; bag F) are higher than conventional stored RBCs after 21 days. These levels are further increased in BTHC and polyolefin bags when the inner bags are enclosed by the gas impermeable outer barrier bag.

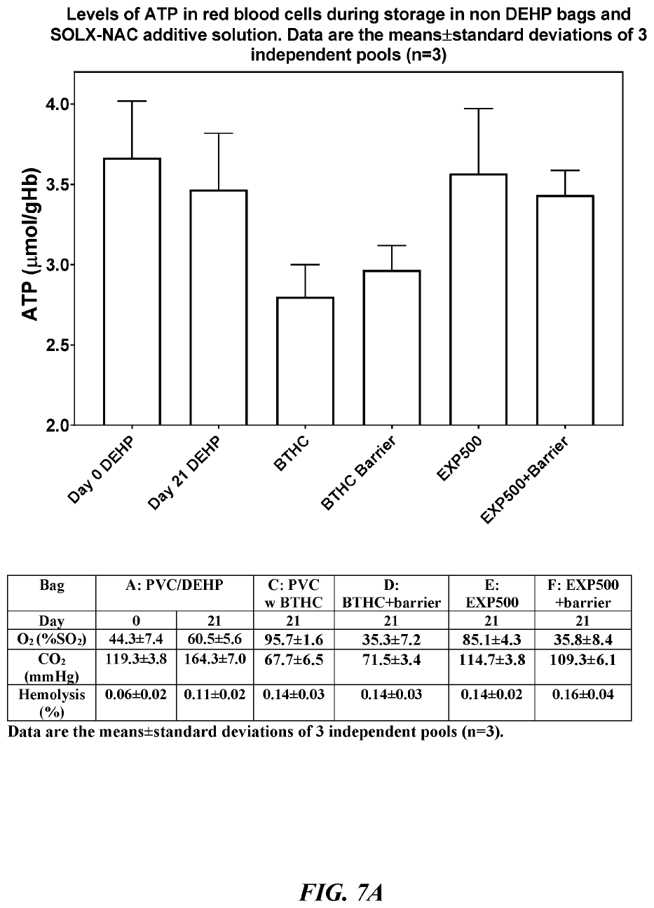

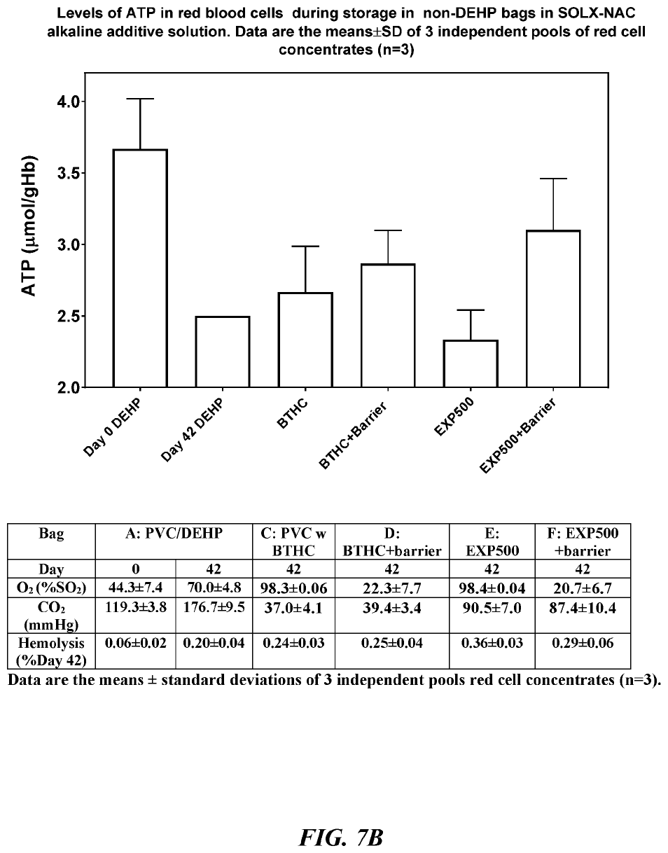

The concentrations of ATP are also significantly higher in BTHC and polyolefin bags with gas impermeable barriers when compared to either the control DEHP or BTHC and polyolefin bags without the barrier (FIG. 7).

Hemolysis remained below the required cut-off levels of 1% and 0.8% established by the U.S and European regulatory authorities, respectively (FIGS. 6 and 7).

TABLE 16
Blood Characteristics in PVC DEHP storage bags
(Bag A with AS7G-NAC solution, control)

| | PVC DEHP (Bag A) | | | |
|---|---|---|---|---|
| | Day | | | |
| In Vitro Metrics | 0 | 21 | 42 | 56 |
| % SO₂ | 44.3 ± 7.4 | 60.5 ± 5.6 | 70.0 ± 4.8 | 75.4 |
| CO₂ (mmHg) | 119.3 ± 3.8 | 164.3 ± 7.0 | 176.7 ± 9.5 | 155 |
| ATP (μmol/g Hb) | 3.7 ± 0.4 | 3.5 ± 0.4 | 2.5 ± 0.0 | 2.1 |
| 2,3DPG (μmol/g Hb) | 11.3 ± 1.5 | 7.8 ± 0.5 | 2.4 ± 0.4 | 1.9 |
| Lactate (mmol/L) | 2.7 ± 0.3 | 16 ± 0.7 | 26.9 ± 0.9 | 31.7 |
| Hemolysis (%) | 0.06 ± 0.02 | 0.11 ± 0.02 | 0.20 ± 0.04 | 0.4 |
| Methemoglobin (%) | 0.60 ± 0.10 | 0.77 ± 0.06 | 1.03 ± 0.06 | 1.3 |
| pH | 7.08 ± 0.03 | 6.87 ± 0.02 | 6.65 ± 0.01 | 6.54 |
| N | 3 | 3 | 3 | 1 |

TABLE 17
Blood Characteristics in BTHC storage bags (Bag E and Bag F with AS7G-NAC solution)

| | | PVC + BTHC (Bag E) | | | PVC + BTHC + Barrier (Bag F) | | |
|---|---|---|---|---|---|---|---|
| Day | 0 | 21 | 42 | 56 | 21 | 42 | 56 |
| In Vitro Metrics | | | | | | | |
| % SO₂ | 44.3 ± 7.4 | 95.7 ± 1.6 | 98.3 ± 0.1 | 97.8 | 35.3 ± 7.2 | 20.7 ± 6.7 | 8.3 |
| CO₂ (mmHg) | 119.3 ± 3.8 | 67.7 ± 6.5 | 37.0 ± 4.1 | 24.5 | 71.5 ± 3.7 | 39.4 ± 3.4 | 22.1 |
| ATP (μmol/g Hb) | 3.7 ± 0.4 | 2.8 ± 0.2 | 2.7 ± 0.3 | 2.3 | 3.0 ± 0.2 | 2.9 ± 0.2 | 3.0 |
| 2,3DPG (μmol/g Hb) | 11.3 ± 1.5 | 14.2 ± 1.5 | 10.8 ± 1.2 | 6.3 | 16.5 ± 2.9 | 12.7 ± 0.7 | 7.0 |
| Lactate (mmol/L) | 2.7 ± 0.3 | 18.7 ± 0.3 | 33.1 ± 0.1 | 39.2 | 20.9 ± 1.1 | 45.2 ± 15.0 | 43.5 |
| Hemolysis (%) | 0.06 ± 0.02 | 0.14 ± 0.03 | 0.24 ± 0.03 | 0.29 | 0.14 ± 0.03 | 0.25 ± 0.04 | 0.37 |
| Methemoglobin (%) | 0.60 ± 0.10 | 0.63 ± 0.15 | 0.93 ± 0.06 | 1 | 0.83 ± 0.06 | 0.87 ± 0.06 | 1.0 |
| pH | 7.08 ± 0.03 | 7.05 ± 0.02 | 6.78 ± 0.03 | 6.58 | 7.05 ± 0.02 | 6.78 ± 0.03 | 6.57 |
| N | 3 | 3 | 3 | 1 | 3 | 3 | 1 |

TABLE 18
Blood Characteristics in EXP500 storage bags (Bag G and Bag H with AS7G-NAC solution)

| | | EXP500 (Polyolefin) (Bag G) | | | EXP500 + Barrier (Bag H) | | |
|---|---|---|---|---|---|---|---|
| Day | 0 | 21 | 42 | 56 | 21 | 42 | 56 |
| In Vitro Metrics | | | | | | | |
| % SO₂ | 44.3 ± 7.4 | 85.1 ± 4.3 | 98.2 ± 0.4 | 98 | 35.8 ± 8.8 | 22.3 ± 7.7 | 10.4 |
| CO₂ (mmHg) | 119.3 ± 3.8 | 114.7 ± 3.8 | 90.5 ± 10.4 | 67.5 | 109.3 ± 6.1 | 85.8 ± 1.6 | 59.9 |
| ATP (μmol/g Hb) | 3.7 ± 0.4 | 3.6 ± 0.4 | 2.3 ± 0.2 | 2.5 | 3.4 ± 0.2 | 3.1 ± 0.4 | 2.9 |
| 2,3DPG (μmol/g Hb) | 11.3 ± 1.5 | 10.1 ± 0.3 | 4.4 ± 0.2 | 3.3 | 12.2 ± 1.7 | 6.4 ± 0.5 | 4.1 |
| Lactate (mmol/L) | 2.7 ± 0.3 | 17.1 ± 0.8 | 30.9 ± 0.4 | 36.2 | 18.4 ± 0.7 | 34.2 ± 1.5 | 38.8 |
| Hemolysis (%) | 0.06 ± 0.02 | 0.14 ± 0.02 | 0.36 ± 0.03 | 0.54 | 0.16 ± 0.04 | 0.29 ± 0.06 | 0.55 |
| Methemoglobin (%) | 0.60 ± 0.10 | 0.73 ± 0.23 | 0.97 ± 0.06 | 1.2 | 0.93 ± 0.06 | 0.97 ± 0.06 | 1 |
| pH | 7.08 ± 0.03 | 6.93 ± 0.02 | 6.69 ± 0.02 | 6.54 | 6.96 ± 0.01 | 6.71 ± 0.01 | 6.57 |
| N | 3 | 3 | 3 | 1 | 3 | 3 | 1 |

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention.

Therefore, it is intended that the invention not be limited to the particular embodiments disclosed as the best mode contemplated for carrying out this invention, but that the invention will include all embodiments falling within the scope and spirit of the appended claims.

1. A method for the storage of a blood product comprising:
obtaining a blood product having a percent oxygen saturation (% SO₂) of greater than 30%;
adding an additive solution to said blood product to prepare a storable blood product; and
storing said storable blood product in a di-2-ethylhexyl phthalate free (DEHP-free) blood compatible (BC) carbon dioxide permeable container having a carbon dioxide permeability of at least 0.62 centimeters cubed per centimeters squared (cm³/cm²) at a pressure of about 1 atmosphere (atm) at 25° C. to prepare a stored blood product.

2. The method of claim 1, wherein said storable blood product is not deoxygenated prior to said storing.

3. The method of claim 1, wherein said storable blood product is not deoxygenated during said storing.

4. The method of claim 2, further comprising depleting oxygen from said storable blood product during said storing.

5. The method of claim 1, wherein said DEHP-free BC carbon dioxide permeable container has an oxygen permeability of less than 0.3 cm³/cm² at a pressure of about 1 atm at 25° C.

6. The method of claim 1, wherein said DEHP-free BC carbon dioxide permeable container does not comprise di(2-ethylhexyl) terephthalate (DEHT).

7. The method of claim 1, wherein said DEHP-free BC carbon dioxide permeable container comprises a plasticizer selected from the group consisting of 1,2-cyclohexane dicarboxylic acid diisononyl ester (DINCH) and butyryltrihexylcitrate (BTHC).

8. The method of claim 1, wherein said DEHP-free BC carbon dioxide permeable container is enclosed within an outer container impermeable to oxygen and carbon dioxide.

9. A container for storing blood comprising a di-2-ethylhexyl phthalate free (DEHP-free) carbon dioxide permeable and oxygen impermeable material, wherein said material has an oxygen permeability for oxygen of less than 0.05 centimeters cubed per centimeters squared (cm³/cm²) and a carbon dioxide permeability of at least 0.62 cm³/cm² at a pressure of about 1 atmosphere (atm) at 25° C.

10. The container of claim 9, wherein said material is selected from the group consisting of polyvinyl chloride (PVC), polyolefin, silicone, polyvinylidene fluoride (PVDF), polysulphone (PS), polypropylene (PP), and polyurethane.

11. The container of claim 9, wherein said material comprises a plasticizer selected from the group consisting of 1,2-cyclohexane dicarboxylic acid diisononyl ester (DINCH) and butyryltrihexylcitrate (BTHC).

12.-16. (canceled)

17. A method for maintaining a level of 2,3-diphosphonoglyceric acid (2,3-DPG) in a blood product comprising:
placing a blood product comprising a percent oxygen saturation (% SO₂) of at least 10% in a storage container comprising
an oxygen and carbon dioxide impermeable outer container enclosing a blood compatible (BC) inner collapsible container having a carbon dioxide permeability of at least 0.62 centimeters cubed per centimeters squared (cm³/cm²) and an oxygen permeability to oxygen of no more than 0.3 cm³/cm² at a pressure of about 1 atmosphere (atm) at 25° C., wherein a carbon dioxide sorbent is disposed between said inner collapsible container and said outer container; and
storing said storage container comprising said blood product for a storage period to prepare a stored blood product,
wherein a level of 2,3-DPG is increased in said stored blood product having been stored for a storage period of up to 14 days compared to a level of 2,3-DPG in a blood product having been conventionally stored for an identical storage period.

18. (canceled)

19. A composition comprising:
a blood product selected from the group consisting of whole blood, platelets, and leukocytes; and
an additive solution comprising sodium bicarbonate (NaHCO₃); sodium phosphate dibasic (Na₂HPO₄); adenine; guanosine; glucose; mannitol; N-acetyl-cysteine; 6-hydroxy-2,5,7,8-tetramethylchroman-2-carboxylic acid (Trolox); and 1-ascorbic acid (vitamin C).

20.-22. (canceled)

23. The method of claim 8, wherein said outer container further encloses a carbon dioxide sorbent disposed between said outer container and said DEHP-free BC carbon dioxide permeable container.

24. The method of claim 1, wherein said additive solution is selected from the group consisting of additive solution 7 (AS-7), AS7G-NAC, AS7G-NAC with 4 mM of gluconate (AS7GG-NAC), additive solution 3 (AS-3) with gluconate, erythrosol-5, erythrosol-5G, and erythrosol-5G with 5 mM Gluconate (erythrosol-5GG).

25. The method of claim 1, wherein said additive solution has a pH of between 7.0 to 8.5.

26. The method of claim 1, wherein said blood product comprises whole blood, platelets, leukocytes, or red blood cells.

27. The method of claim 1, wherein said stored blood product has an SO₂ of greater than 15% during up to 42 days of storage.

28. The method of claim 1, wherein said stored blood product has a partial pressure of carbon dioxide (pCO₂) of less than 125 millimeters of mercury (mmHg) during up to 42 days of storage.

29. The method of claim 17, wherein a level of adenosine triphosphate (ATP) is increased in said stored blood product after having been stored for a storage period of at least 42 days compared to a level of ATP in a blood product having been conventionally stored for an identical storage period.
